SEQUENCE LISTING (172 sequences)

SEQ ID NO: 1 - skipped

SEQ ID NO: 2 - DNA, 10 bp, Artificial Sequence
  Synthetic-CC(A/T)6GG element (CArG)
ccaaatttgg

SEQ ID NO: 3-7 - skipped (5 entries)

SEQ ID NO: 8 - DNA, 10 bp, Artificial Sequence
  Synthetic-E4F1
gtgacgtaac

SEQ ID NO: 9 - skipped

SEQ ID NO: 10 - DNA, 15 bp, Artificial Sequence
  Synthetic-Estrogen-related receptor alpha RE (ERRE)
aggtcattttgacct

SEQ ID NO: 11-13 - skipped (3 entries)

SEQ ID NO: 14 - DNA, 15 bp, Artificial Sequence
  Synthetic-Glucocorticoid RE (GRE)
agaacattttgttct

SEQ ID NO: 15 - skipped

SEQ ID NO: 16 - DNA, 11 bp, Artificial Sequence
  Synthetic-Helios RE (HRE)
aatagggactt

SEQ ID NO: 17 - DNA, 13 bp, Artificial Sequence
  Synthetic-Hepatocyte nuclear factor 1 (HNF)
gggccaaaggtct

SEQ ID NO: 18 - DNA, 12 bp, Artificial Sequence
  Synthetic-Insulin promoter factor 1 (IPF1)
cccattagggac

SEQ ID NO: 19 - DNA, 13 bp, Artificial Sequence
  Synthetic-Interferon-stimulated RE (ISRE)
gaaaagtgaaacc

SEQ ID NO: 20 - DNA, 10 bp, Artificial Sequence
  Synthetic-Myocyte enhancer factor 2 (MEF2)
ctaaaaatag

SEQ ID NO: 21-22 - skipped (2 entries)

SEQ ID NO: 23 - DNA, 15 bp, Artificial Sequence
  Synthetic-Nuclear factor 1 (NF1)
ttggctatatgccaa

SEQ ID NO: 24 - skipped

SEQ ID NO: 25 - DNA, 10 bp, Artificial Sequence
  Synthetic-Nuclear factor kappa B (NF?B)
gggactttcc

SEQ ID NO: 26 - skipped

SEQ ID NO: 27 - DNA, 17 bp, Artificial Sequence
  Synthetic-Retinoic acid RE (RARE)
aggtcatcaagaggtca

SEQ ID NO: 28-29 - skipped (2 entries)

SEQ ID NO: 30 - DNA, 210 bp, Artificial Sequence
  Synthetic-1/01
tgggactttccatcgaaacacgtgaatcgaaacacgtgaatcgatgggactttccatcgatggggcggggatcgattttgcgcaatttcgatttgacgtcatttcgattcacgtgtttcgaaattgcgcaaaatcgatgggactttccatcgatgggactttccatcgaaacacgtgaatcgatccccgccccatcgatgggactttcca

SEQ ID NO: 31 - DNA, 232 bp, Artificial Sequence
  Synthetic-1/02
ttcacgtgtttcgattgtgacgtaactttcgaaagttacgtcacaatcgaaattgcgcaaaatcgaaacacgtgaatcgatccccgccccatcgatggaaagtcccatcgattcacgtgtttcgattttgcgcaatttcgatgggactttccatcgatggaaagtcccatcgatgggactttccatcgatggaaagtcccatcgatggggcggggatcgatgggactttcca

SEQ ID NO: 32 - DNA, 238 bp, Artificial Sequence
  Synthetic-1/03
aacacgtgaatcgaaacacgtgaatcgatccccgccccatcgatttgacgtcatttcgatggaaagtcccatcgatccccgccccatcgatggggcggggatcgatccccgccccatcgaaatgacgtcaaatcgaaacacgtgaatcgatgggactttccatcgatggaaagtcccatcgaaattgcgcaaaatcgattcacgtgtttcgattttgcgcaatttcgattcacgtgtt

SEQ ID NO: 33 - DNA, 202 bp, Artificial Sequence
  Synthetic-1/04
aattgcgcaaaatcgaaatgacgtcaaatcgattcacgtgtttcgatggggcggggatcgatttgacgtcatttcgaaattgcgcaaaatcgatgggactttccatcgatggaaagtcccatcgatggaaagtcccatcgatggggcggggatcgaaagttacgtcacaatcgattttgcgcaatttcgatggaaagtccca

SEQ ID NO: 34 - DNA, 170 bp, Artificial Sequence
  Synthetic-1/05
tggggcggggatcgattgtgacgtaactttcgatgggactttccatcgatggggcggggatcgatgggactttccatcgaaattgcgcaaaatcgatggaaagtcccatcgattttgcgcaatttcgatttgacgtcatttcgattcacgtgtttcgatggaaagtccca

SEQ ID NO: 35 - DNA, 193 bp, Artificial Sequence
  Synthetic-1/06
tttgacgtcatttcgaaacacgtgaatcgattcacgtgtttcgatccccgccccatcgaaacacgtgaatcgatgggactttccatcgaaacacgtgaatcgatttgacgtcatttcgaaattgcgcaaaatcgatccccgccccatcgaaatgacgtcaaatcgatggaaagtcccatcgatccccgcccca

SEQ ID NO: 36 - DNA, 167 bp, Artificial Sequence
  Synthetic-1/07
aattgcgcaaaatcgaaacacgtgaatcgaaattgcgcaaaatcgatccccgccccatcgatgggactttccatcgattttgcgcaatttcgattttgcgcaatttcgaaacacgtgaatcgaaatgacgtcaaatcgatgggactttccatcgatgggactttcca

SEQ ID NO: 37 - DNA, 161 bp, Artificial Sequence
  Synthetic-1/08
aacacgtgaatcgattcacgtgtttcgatggaaagtcccatcgatttgacgtcatttcgaaattgcgcaaaatcgattcacgtgtttcgaaacacgtgaatcgatggggcggggatcgattttgcgcaatttcgaaacacgtgaatcgatgggactttcca

SEQ ID NO: 38 - DNA, 164 bp, Artificial Sequence
  Synthetic-1/09
ttttgcgcaatttcgatccccgccccatcgatgggactttccatcgaaattgcgcaaaatcgaaatgacgtcaaatcgatggaaagtcccatcgaaacacgtgaatcgattcacgtgtttcgaaatgacgtcaaatcgatggggcggggatcgattcacgtgtt

SEQ ID NO: 39 - DNA, 148 bp, Artificial Sequence
  Synthetic-1/10
aattgcgcaaaatcgaaacacgtgaatcgatggaaagtcccatcgaaacacgtgaatcgattcacgtgtttcgattttgcgcaatttcgaaatgacgtcaaatcgatggggcggggatcgatggggcggggatcgatgggactttcca

SEQ ID NO: 40 - DNA, 105 bp, Artificial Sequence
  Synthetic-1/11
aatgacgtcaaatcgattcacgtgtttcgatgggactttccatcgaaattgcgcaaaatcgaaacacgtgaatcgatccccgccccatcgaaagttacgtcacaa

SEQ ID NO: 41 - DNA, 165 bp, Artificial Sequence
  Synthetic-1/12
aattgcgcaaaatcgatgggactttccatcgatgggactttccatcgaaacacgtgaatcgaaacacgtgaatcgatccccgccccatcgatttgacgtcatttcgaaacacgtgaatcgatggaaagtcccatcgaaattgcgcaaaatcgattttgcgcaatt

SEQ ID NO: 42 - DNA, 208 bp, Artificial Sequence
  Synthetic-1/13
tggggcggggatcgattttgcgcaatttcgattcacgtgtttcgatggaaagtcccatcgatggggcggggatcgaaacacgtgaatcgatgggactttccatcgaaatgacgtcaaatcgaaattgcgcaaaatcgatgggactttccatcgattcacgtgtttcgattcacgtgtttcgatttgacgtcatttcgattcacgtgtt

SEQ ID NO: 43 - DNA, 202 bp, Artificial Sequence
  Synthetic-1/14
tttgacgtcatttcgatggggcggggatcgatggggcggggatcgatgggactttccatcgatttgacgtcatttcgattttgcgcaatttcgaaattgcgcaaaatcgatggaaagtcccatcgatgggactttccatcgaaatgacgtcaaatcgattgtgacgtaactttcgattcacgtgtttcgatgggactttcca

SEQ ID NO: 44 - DNA, 156 bp, Artificial Sequence
  Synthetic-1/15
tggaaagtcccatcgattttgcgcaatttcgaaagttacgtcacaatcgaaatgacgtcaaatcgatggaaagtcccatcgattttgcgcaatttcgattcacgtgtttcgatgggactttccatcgaaatgacgtcaaatcgatggaaagtccca

SEQ ID NO: 45 - DNA, 140 bp, Artificial Sequence
  Synthetic-1/16
tttgacgtcatttcgaaagttacgtcacaatcgatgggactttccatcgatgggactttccatcgaaattgcgcaaaatcgaaatgacgtcaaatcgaaacacgtgaatcgattcacgtgtttcgaaagttacgtcacaa

SEQ ID NO: 46 - DNA, 211 bp, Artificial Sequence
  Synthetic-1/17
tccccgccccatcgatggggcggggatcgatggggcggggatcgaaattgcgcaaaatcgaaacacgtgaatcgatgggactttccatcgattttgcgcaatttcgatggaaagtcccatcgaaattgcgcaaaatcgatttgacgtcatttcgaaattgcgcaaaatcgaaacacgtgaatcgatccccgccccatcgatggggcgggga

SEQ ID NO: 47 - DNA, 135 bp, Artificial Sequence
  Synthetic-1/18
tgggactttccatcgatggaaagtcccatcgaaacacgtgaatcgaaattgcgcaaaatcgatttgacgtcatttcgatccccgccccatcgaaacacgtgaatcgaaattgcgcaaaatcgaaatgacgtcaaa

SEQ ID NO: 48 - DNA, 198 bp, Artificial Sequence
  Synthetic-1/19
aacacgtgaatcgaaacacgtgaatcgatttgacgtcatttcgaaacacgtgaatcgatttgacgtcatttcgatgggactttccatcgatggaaagtcccatcgattgtgacgtaactttcgatttgacgtcatttcgatgggactttccatcgattttgcgcaatttcgatggaaagtcccatcgattcacgtgtt

SEQ ID NO: 49 - DNA, 178 bp, Artificial Sequence
  Synthetic-1/20
aagttacgtcacaatcgatggggcggggatcgaaacacgtgaatcgaaacacgtgaatcgattcacgtgtttcgatggaaagtcccatcgattgtgacgtaactttcgaaacacgtgaatcgattttgcgcaatttcgatccccgccccatcgattcacgtgtttcgaaacacgtgaa

SEQ ID NO: 50 - DNA, 104 bp, Artificial Sequence
  Synthetic-1/21
tgggactttccatcgaaacacgtgaatcgatggaaagtcccatcgattcacgtgtttcgatgggactttccatcgatgggactttccatcgatggaaagtccca

SEQ ID NO: 51 - DNA, 165 bp, Artificial Sequence
  Synthetic-1/22
ttgtgacgtaactttcgatccccgccccatcgaaattgcgcaaaatcgaaacacgtgaatcgatggaaagtcccatcgattcacgtgtttcgaaattgcgcaaaatcgattcacgtgtttcgatggggcggggatcgatggggcggggatcgattttgcgcaatt

SEQ ID NO: 52 - DNA, 135 bp, Artificial Sequence
  Synthetic-1/23
aatgacgtcaaatcgatgggactttccatcgattcacgtgtttcgattttgcgcaatttcgatttgacgtcatttcgattttgcgcaatttcgatgggactttccatcgatccccgccccatcgaaacacgtgaa

SEQ ID NO: 53 - DNA, 169 bp, Artificial Sequence
  Synthetic-1/24
tttgacgtcatttcgatggaaagtcccatcgattttgcgcaatttcgattttgcgcaatttcgattcacgtgtttcgattgtgacgtaactttcgatgggactttccatcgattcacgtgtttcgatggggcggggatcgatggaaagtcccatcgatttgacgtcatt

SEQ ID NO: 54 - DNA, 229 bp, Artificial Sequence
  Synthetic-1/25
tggggcggggatcgatccccgccccatcgaaattgcgcaaaatcgatccccgccccatcgaaattgcgcaaaatcgatggaaagtcccatcgatttgacgtcatttcgatgggactttccatcgatgggactttccatcgaaattgcgcaaaatcgattcacgtgtttcgatttgacgtcatttcgatttgacgtcatttcgattcacgtgtttcgaaattgcgcaaaa

SEQ ID NO: 55 - DNA, 183 bp, Artificial Sequence
  Synthetic-1/26
tgggactttccatcgatccccgccccatcgaaattgcgcaaaatcgatgggactttccatcgaaacacgtgaatcgaaacacgtgaatcgattcacgtgtttcgaaagttacgtcacaatcgatgggactttccatcgaaattgcgcaaaatcgatggaaagtcccatcgattttgcgcaatt

SEQ ID NO: 56 - DNA, 171 bp, Artificial Sequence
  Synthetic-1/27
aattgcgcaaaatcgatttgacgtcatttcgaaacacgtgaatcgaaagttacgtcacaatcgaaattgcgcaaaatcgaaacacgtgaatcgaaagttacgtcacaatcgatgggactttccatcgatggggcggggatcgatgggactttccatcgatgggactttcca

SEQ ID NO: 57 - DNA, 325 bp, Artificial Sequence
  Synthetic-1/28
ttcacgtgtttcgattgtgacgtaactttcgatggaaagtcccatcgatgggactttccatcgaaacacgtgaatcgaaacacgtgaatcgatttgacgtcatttcgatgggactttccatcgattcacgtgtttcgaaagttacgtcacaatcgatgggactttccatcgatccccgccccatcgatgggactttccatcgatggggcggggatcgattcacgtgtttcgattgtgacgtaactttcgatgggactttccatcgatccccgccccatcgatgggactttccatcgattcacgtgtttcgaaagttacgtcacaa

SEQ ID NO: 58 - DNA, 96 bp, Artificial Sequence
  Synthetic-1/29
tgggactttccatcgatgggactttccatcgatgggactttccatcgatgggactttccatcgatgggactttccatcgatgggactttccatcga

SEQ ID NO: 59 - DNA, 103 bp, Artificial Sequence
  Synthetic-1/30
tttgacgtcatttcgaaattgcgcaaaatcgatgggactttccatcgattcacgtgtttcgattcacgtgtttcgatggggcggggatcgatggaaagtccca

SEQ ID NO: 60 - DNA, 184 bp, Artificial Sequence
  Synthetic-1/31
aattgcgcaaaatcgatggggcggggatcgattttgcgcaatttcgattcacgtgtttcgaaagttacgtcacaatcgatggaaagtcccatcgatttgacgtcatttcgattttgcgcaatttcgatggaaagtcccatcgattcacgtgtttcgattttgcgcaatttcgatggggcgggga

SEQ ID NO: 61 - DNA, 245 bp, Artificial Sequence
  Synthetic-1/32
ttcacgtgtttcgattgtgacgtaactttcgatccccgccccatcgatgggactttccatcgatggggcggggatcgaaacacgtgaatcgaaacacgtgaatcgatgggactttccatcgattgtgacgtaactttcgatgggactttccatcgatttgacgtcatttcgatggggcggggatcgaaattgcgcaaaatcgatggggcggggatcgatggggcggggatcgaaattgcgcaaaa

SEQ ID NO: 62 - DNA, 221 bp, Artificial Sequence
  Synthetic-1/33
tttgacgtcatttcgatggaaagtcccatcgattgtgacgtaactttcgatgggactttccatcgatccccgccccatcgatccccgccccatcgatccccgccccatcgatggaaagtcccatcgattgtgacgtaactttcgattgtgacgtaactttcgaaatgacgtcaaatcgatgggactttccatcgattcacgtgtttcgatggaaagtccca

SEQ ID NO: 63 - DNA, 213 bp, Artificial Sequence
  Synthetic-1/34
tggggcggggatcgatccccgccccatcgatggaaagtcccatcgaaacacgtgaatcgaaacacgtgaatcgattgtgacgtaactttcgatttgacgtcatttcgaaacacgtgaatcgaaattgcgcaaaatcgattcacgtgtttcgaaatgacgtcaaatcgatggaaagtcccatcgattgtgacgtaactttcgatccccgcccca

SEQ ID NO: 64 - DNA, 196 bp, Artificial Sequence
  Synthetic-1/3
tttgacgtcatttcgattttgcgcaatttcgaaatgacgtcaaatcgaaacacgtgaatcgattttgcgcaatttcgatccccgccccatcgatggaaagtcccatcgaaattgcgcaaaatcgattcacgtgtttcgatggggcggggatcgatggaaagtcccatcgatggggcggggatcgatggggcgggga

SEQ ID NO: 65 - DNA, 203 bp, Artificial Sequence
  Synthetic-1/36
aacacgtgaatcgaaatgacgtcaaatcgatgggactttccatcgatggggcggggatcgaaagttacgtcacaatcgattgtgacgtaactttcgatggggcggggatcgattgtgacgtaactttcgatccccgccccatcgatgggactttccatcgaaacacgtgaatcgattttgcgcaatttcgaaatgacgtcaaa

SEQ ID NO: 66 - DNA, 318 bp, Artificial Sequence
  Synthetic-1/37
tgggactttccatcgaaagttacgtcacaatcgaaacacgtgaatcgaaacacgtgaatcgattgtgacgtaactttcgaaagttacgtcacaatcgattttgcgcaatttcgatgggactttccatcgattttgcgcaatttcgatgggactttccatcgattgtgacgtaactttcgatggggcggggatcgaaagttacgtcacaatcgatttgacgtcatttcgaaatgacgtcaaatcgatggggcggggatcgatggaaagtcccatcgaaacacgtgaatcgatttgacgtcatttcgatggaaagtccca

SEQ ID NO: 67 - DNA, 169 bp, Artificial Sequence
  Synthetic-1/38
tggaaagtcccatcgatttgacgtcatttcgatgggactttccatcgatttgacgtcatttcgatttgacgtcatttcgattcacgtgtttcgatggggcggggatcgatttgacgtcatttcgaaattgcgcaaaatcgatgggactttccatcgatgggactttcca

SEQ ID NO: 68 - DNA, 211 bp, Artificial Sequence
  Synthetic-1/39
aatgacgtcaaatcgattcacgtgtttcgaaatgacgtcaaatcgattcacgtgtttcgattgtgacgtaactttcgattttgcgcaatttcgatccccgccccatcgatccccgccccatcgatggaaagtcccatcgatggggcggggatcgattcacgtgtttcgattcacgtgtttcgatttgacgtcatttcgatttgacgtcatt

SEQ ID NO: 69 - DNA, 361 bp, Artificial Sequence
  Synthetic-1/40
ttcacgtgtttcgattgtgacgtaactttcgatggggcggggatcgatttgacgtcatttcgaaacacgtgaatcgattgtgacgtaactttcgaaagttacgtcacaatcgatccccgccccatcgatggggcggggatcgaaagttacgtcacaatcgatggggcggggatcgattgtgacgtaactttcgatggggcggggatcgaaacacgtgaatcgatggggcggggatcgaaattgcgcaaaatcgatgggactttccatcgaaattgcgcaaaatcgaaacacgtgaatcgatgggactttccatcgattgtgacgtaactttcgattttgcgcaatttcgatccccgcccca

SEQ ID NO: 70 - DNA, 186 bp, Artificial Sequence
  Synthetic-1/41
aattgcgcaaaatcgaaattgcgcaaaatcgaaacacgtgaatcgatccccgccccatcgaaagttacgtcacaatcgatccccgccccatcgaaattgcgcaaaatcgaaagttacgtcacaatcgaaacacgtgaatcgatggaaagtcccatcgattttgcgcaatttcgatttgacgtcatt

SEQ ID NO: 71 - DNA, 311 bp, Artificial Sequence
  Synthetic-1/42
aagttacgtcacaatcgaaattgcgcaaaatcgatccccgccccatcgatgggactttccatcgattttgcgcaatttcgattttgcgcaatttcgaaagttacgtcacaatcgattcacgtgtttcgaaacacgtgaatcgattttgcgcaatttcgaaatgacgtcaaatcgatgggactttccatcgattttgcgcaatttcgattcacgtgtttcgatgggactttccatcgatccccgccccatcgaaattgcgcaaaatcgatttgacgtcatttcgatggaaagtcccatcgatggggcgggga

SEQ ID NO: 72 - DNA, 135 bp, Artificial Sequence
  Synthetic-1/43
tggggcggggatcgattcacgtgtttcgattgtgacgtaactttcgatgggactttccatcgatttgacgtcatttcgattttgcgcaatttcgaaatgacgtcaaatcgaaacacgtgaatcgaaacacgtgaa

SEQ ID NO: 73 - DNA, 135 bp, Artificial Sequence
  Synthetic-1/44
tccccgccccatcgaaacacgtgaatcgaaacacgtgaatcgattgtgacgtaactttcgaaatgacgtcaaatcgaaatgacgtcaaatcgaaattgcgcaaaatcgatgggactttccatcgattcacgtgtt

SEQ ID NO: 74 - DNA, 184 bp, Artificial Sequence
  Synthetic-1/45
aagttacgtcacaatcgattttgcgcaatttcgatggaaagtcccatcgaaatgacgtcaaatcgattttgcgcaatttcgaaatgacgtcaaatcgatggggcggggatcgatggaaagtcccatcgattcacgtgtttcgatggggcggggatcgaaacacgtgaatcgatttgacgtcatt

SEQ ID NO: 75 - DNA, 271 bp, Artificial Sequence
  Synthetic-1/46
tgggactttccatcgatggggcggggatcgattcacgtgtttcgaaatgacgtcaaatcgatccccgccccatcgaaacacgtgaatcgattttgcgcaatttcgattcacgtgtttcgaaacacgtgaatcgatttgacgtcatttcgattgtgacgtaactttcgaaatgacgtcaaatcgattcacgtgtttcgaaacacgtgaatcgattgtgacgtaactttcgaaacacgtgaatcgatggggcggggatcgatttgacgtcatt

SEQ ID NO: 76 - DNA, 98 bp, Artificial Sequence
  Synthetic-1/47
aacacgtgaatcgaaacacgtgaatcgaaacacgtgaatcgaaacacgtgaatcgaaacacgtgaatcgaaacacgtgaatcgaaacacgtgaatcga

SEQ ID NO: 77 - DNA, 228 bp, Artificial Sequence
  Synthetic-1/48
ttcacgtgtttcgatccccgccccatcgattgtgacgtaactttcgatccccgccccatcgattcacgtgtttcgatttgacgtcatttcgattgtgacgtaactttcgatccccgccccatcgattcacgtgtttcgatggggcggggatcgaaattgcgcaaaatcgattcacgtgtttcgattttgcgcaatttcgattttgcgcaatttcgatttgacgtcatt

SEQ ID NO: 78 - DNA, 217 bp, Artificial Sequence
  Synthetic-1/49
aattgcgcaaaatcgattcacgtgtttcgattgtgacgtaactttcgatccccgccccatcgatgggactttccatcgattgtgacgtaactttcgatttgacgtcatttcgatggggcggggatcgatccccgccccatcgaaattgcgcaaaatcgatggaaagtcccatcgaaattgcgcaaaatcgaaacacgtgaatcgatttgacgtcatt

SEQ ID NO: 79 - DNA, 89 bp, Artificial Sequence
  Synthetic-1/50
ttgtgacgtaactttcgatccccgccccatcgattcacgtgtttcgattcacgtgtttcgaaatgacgtcaaatcgaaattgcgcaaaa

SEQ ID NO: 80 - DNA, 202 bp, Artificial Sequence
  Synthetic-1/51
ttgtgacgtaactttcgaaatgacgtcaaatcgatccccgccccatcgatgggactttccatcgaaagttacgtcacaatcgatttgacgtcatttcgatggggcggggatcgatgggactttccatcgatggggcggggatcgaaatgacgtcaaatcgattttgcgcaatttcgaaacacgtgaatcgatccccgcccca

SEQ ID NO: 81 - DNA, 336 bp, Artificial Sequence
  Synthetic-1/52
tttgacgtcatttcgatggaaagtcccatcgaaatgacgtcaaatcgatgggactttccatcgaaatgacgtcaaatcgatccccgccccatcgatggaaagtcccatcgattcacgtgtttcgatggaaagtcccatcgattgtgacgtaactttcgatttgacgtcatttcgatttgacgtcatttcgatggaaagtcccatcgaaattgcgcaaaatcgaaatgacgtcaaatcgatttgacgtcatttcgatgggactttccatcgaaatgacgtcaaatcgatggaaagtcccatcgattgtgacgtaactttcgatggggcggggatcga

SEQ ID NO: 82 - DNA, 156 bp, Artificial Sequence
  Synthetic-1/53
tggggcggggatcgatccccgccccatcgaaagttacgtcacaatcgatgggactttccatcgaaattgcgcaaaatcgatggaaagtcccatcgaaagttacgtcacaatcgattcacgtgtttcgaaattgcgcaaaatcgatgggactttcca

SEQ ID NO: 83 - DNA, 112 bp, Artificial Sequence
  Synthetic-1/54
tttgacgtcatttcgatttgacgtcatttcgatttgacgtcatttcgatttgacgtcatttcgatttgacgtcatttcgatttgacgtcatttcgatttgacgtcatttcga

SEQ ID NO: 84 - DNA, 105 bp, Artificial Sequence
  Synthetic-1/55
tccccgccccatcgatttgacgtcatttcgaaatgacgtcaaatcgatttgacgtcatttcgatggaaagtcccatcgaaacacgtgaatcgatgggactttcca

SEQ ID NO: 85 - DNA, 138 bp, Artificial Sequence
  Synthetic-1/56
tccccgccccatcgaaattgcgcaaaatcgaaatgacgtcaaatcgatgggactttccatcgattcacgtgtttcgatggggcggggatcgaaacacgtgaatcgaaagttacgtcacaatcgattgtgacgtaactt

SEQ ID NO: 86 - DNA, 140 bp, Artificial Sequence
  Synthetic-1/57
tggaaagtcccatcgaaattgcgcaaaatcgattttgcgcaatttcgatccccgccccatcgattgtgacgtaactttcgatccccgccccatcgattgtgacgtaactttcgatgggactttccatcgaaacacgtgaa

SEQ ID NO: 87 - DNA, 59 bp, Artificial Sequence
  Synthetic-1/58
tgggactttccatcgatggggcggggatcgaaatgacgtcaaatcgatttgacgtcatt

SEQ ID NO: 88 - DNA, 217 bp, Artificial Sequence
  Synthetic-1/59
aatgacgtcaaatcgatccccgccccatcgatggggcggggatcgattcacgtgtttcgattcacgtgtttcgatccccgccccatcgattttgcgcaatttcgatgggactttccatcgattttgcgcaatttcgatgggactttccatcgaaagttacgtcacaatcgattgtgacgtaactttcgaaacacgtgaatcgaaagttacgtcacaa

SEQ ID NO: 89 - DNA, 101 bp, Artificial Sequence
  Synthetic-1/60
tggggcggggatcgatggggcggggatcgatggggcggggatcgatggggcggggatcgatggggcggggatcgatggggcggggatcgatggggcgggga

SEQ ID NO: 90 - DNA, 135 bp, Artificial Sequence
  Synthetic-1/61
tccccgccccatcgaaattgcgcaaaatcgatttgacgtcatttcgatttgacgtcatttcgatggaaagtcccatcgaaattgcgcaaaatcgaaacacgtgaatcgaaacacgtgaatcgaaattgcgcaaaa

SEQ ID NO: 91 - DNA, 108 bp, Artificial Sequence
  Synthetic-1/62
ttgtgacgtaactttcgattgtgacgtaactttcgattgtgacgtaactttcgattgtgacgtaactttcgattgtgacgtaactttcgattgtgacgtaactttcga

SEQ ID NO: 92 - DNA, 262 bp, Artificial Sequence
  Synthetic-1/63
ttcacgtgtttcgaaacacgtgaatcgatggggcggggatcgatggggcggggatcgaaatgacgtcaaatcgatggaaagtcccatcgatggggcggggatcgaaattgcgcaaaatcgatttgacgtcatttcgatggggcggggatcgaaagttacgtcacaatcgaaagttacgtcacaatcgattttgcgcaatttcgaaacacgtgaatcgaaacacgtgaatcgattttgcgcaatttcgaaagttacgtcacaa

SEQ ID NO: 93 - DNA, 137 bp, Artificial Sequence
  Synthetic-1/64
aacacgtgaatcgattttgcgcaatttcgattttgcgcaatttcgaaatgacgtcaaatcgaaagttacgtcacaatcgatttgacgtcatttcgattttgcgcaatttcgattcacgtgtttcgatccccgcccca

SEQ ID NO: 94 - DNA, 139 bp, Artificial Sequence
  Synthetic-1/65
aagttacgtcacaatcgattttgcgcaatttcgatccccgccccatcgaaattgcgcaaaatcgatttgacgtcatttcgaaacacgtgaatcgattcacgtgtttcgaaagttacgtcacaatcgattttgcgcaatt

SEQ ID NO: 95 - DNA, 214 bp, Artificial Sequence
  Synthetic-1/66
ttcacgtgtttcgaaacacgtgaatcgatttgacgtcatttcgatggggcggggatcgatgggactttccatcgaaatgacgtcaaatcgaaagttacgtcacaatcgaaacacgtgaatcgatttgacgtcatttcgatccccgccccatcgaaacacgtgaatcgaaagttacgtcacaatcgatgggactttccatcgatttgacgtcatt

SEQ ID NO: 96 - DNA, 189 bp, Artificial Sequence
  Synthetic-1/67
ttcacgtgtttcgatccccgccccatcgatccccgccccatcgatgggactttccatcgattgtgacgtaactttcgattcacgtgtttcgaaagttacgtcacaatcgatgggactttccatcgatggaaagtcccatcgatggggcggggatcgattgtgacgtaactttcgaaagttacgtcacaa

SEQ ID NO: 97 - DNA, 488 bp, Artificial Sequence
  Synthetic-1/68
tccccgccccatcgatccccgccccatcgatggaaagtcccatcgaaagttacgtcacaatcgatggaaagtcccatcgatttgacgtcatttcgaaattgcgcaaaatcgattcacgtgtttcgaaatgacgtcaaatcgaaacacgtgaatcgattttgcgcaatttcgattgtgacgtaactttcgattcacgtgtttcgattgtgacgtaactttcgatggaaagtcccatcgatggggcggggatcgaaatgacgtcaaatcgatggggcggggatcgaaatgacgtcaaatcgatgggactttccatcgatccccgccccatcgatttgacgtcatttcgattttgcgcaatttcgatttgacgtcatttcgattgtgacgtaactttcgatggaaagtcccatcgattttgcgcaatttcgaaacacgtgaatcgattgtgacgtaactttcgaaattgcgcaaaatcgatggggcgggga

SEQ ID NO: 98 - DNA, 154 bp, Artificial Sequence
  Synthetic-1/69
aacacgtgaatcgatggggcggggatcgattttgcgcaatttcgatttgacgtcatttcgaaagttacgtcacaatcgattttgcgcaatttcgattttgcgcaatttcgatggggcggggatcgatgggactttccatcgatttgacgtcatt

SEQ ID NO: 99 - DNA, 230 bp, Artificial Sequence
  Synthetic-1/70
ttcacgtgtttcgatggaaagtcccatcgatggggcggggatcgattgtgacgtaactttcgatgggactttccatcgattcacgtgtttcgatccccgccccatcgatttgacgtcatttcgattgtgacgtaactttcgattcacgtgtttcgaaatgacgtcaaatcgatggggcggggatcgatggggcggggatcgatttgacgtcatttcgaaattgcgcaaaa

SEQ ID NO: 100 - DNA, 171 bp, Artificial Sequence
  Synthetic-1/71
ttcacgtgtttcgatggaaagtcccatcgattgtgacgtaactttcgatccccgccccatcgatttgacgtcatttcgattgtgacgtaactttcgaaacacgtgaatcgattttgcgcaatttcgaaatgacgtcaaatcgatttgacgtcatttcgatgggactttcca

SEQ ID NO: 101 - DNA, 366 bp, Artificial Sequence
  Synthetic-1/72
ttgtgacgtaactttcgaaagttacgtcacaatcgatttgacgtcatttcgatccccgccccatcgaaatgacgtcaaatcgaaattgcgcaaaatcgaaatgacgtcaaatcgatggaaagtcccatcgatttgacgtcatttcgatggggcggggatcgattttgcgcaatttcgattttgcgcaatttcgaaagttacgtcacaatcgatttgacgtcatttcgatggaaagtcccatcgattgtgacgtaactttcgattcacgtgtttcgaaacacgtgaatcgaaattgcgcaaaatcgaaagttacgtcacaatcgatccccgccccatcgatggggcggggatcgaaattgcgcaaaa

SEQ ID NO: 102 - DNA, 108 bp, Artificial Sequence
  Synthetic-1/73
ttttgcgcaatttcgattttgcgcaatttcgattttgcgcaatttcgattttgcgcaatttcgattttgcgcaatttcgattttgcgcaatttcgattttgcgcaatt

SEQ ID NO: 103 - DNA, 169 bp, Artificial Sequence
  Synthetic-1/74
aatgacgtcaaatcgatccccgccccatcgaaatgacgtcaaatcgaaattgcgcaaaatcgattttgcgcaatttcgaaacacgtgaatcgaaattgcgcaaaatcgaaatgacgtcaaatcgattttgcgcaatttcgattttgcgcaatttcgattttgcgcaatt

SEQ ID NO: 104 - DNA, 156 bp, Artificial Sequence
  Synthetic-1/75
tggggcggggatcgatggggcggggatcgatggaaagtcccatcgatggaaagtcccatcgattgtgacgtaactttcgatggggcggggatcgattgtgacgtaactttcgaaatgacgtcaaatcgattttgcgcaatttcgatccccgcccca

SEQ ID NO: 105 - DNA, 172 bp, Artificial Sequence
  Synthetic-1/76
ttttgcgcaatttcgattgtgacgtaactttcgatggggcggggatcgatggggcggggatcgatggggcggggatcgatggggcggggatcgattgtgacgtaactttcgattttgcgcaatttcgaaatgacgtcaaatcgaaacacgtgaatcgattgtgacgtaactt

SEQ ID NO: 106 - DNA, 238 bp, Artificial Sequence
  Synthetic-1/77
aacacgtgaatcgattgtgacgtaactttcgattcacgtgtttcgatgggactttccatcgattgtgacgtaactttcgatccccgccccatcgatgggactttccatcgattgtgacgtaactttcgatgggactttccatcgatccccgccccatcgattgtgacgtaactttcgaaatgacgtcaaatcgattgtgacgtaactttcgatccccgccccatcgatggggcgggga

SEQ ID NO: 107 - DNA, 140 bp, Artificial Sequence
  Synthetic-1/78
aagttacgtcacaatcgatggggcggggatcgatggggcggggatcgaaatgacgtcaaatcgatggggcggggatcgatccccgccccatcgattgtgacgtaactttcgatggaaagtcccatcgaaattgcgcaaaa

SEQ ID NO: 108 - DNA, 210 bp, Artificial Sequence
  Synthetic-1/79
ttgtgacgtaactttcgattgtgacgtaactttcgaaagttacgtcacaatcgattcacgtgtttcgaaagttacgtcacaatcgaaatgacgtcaaatcgattgtgacgtaactttcgattttgcgcaatttcgaaatgacgtcaaatcgatgggactttccatcgattttgcgcaatttcgaaacacgtgaatcgaaattgcgcaaaa

SEQ ID NO: 109 - DNA, 151 bp, Artificial Sequence
  Synthetic-1/80
ttcacgtgtttcgatggggcggggatcgatttgacgtcatttcgaaacacgtgaatcgaaagttacgtcacaatcgaaagttacgtcacaatcgaaattgcgcaaaatcgatggggcggggatcgattcacgtgtttcgatggggcgggga

SEQ ID NO: 110 - DNA, 125 bp, Artificial Sequence
  Synthetic-1/81
aattgcgcaaaatcgatggggcggggatcgatttgacgtcatttcgaaattgcgcaaaatcgatggggcggggatcgattgtgacgtaactttcgatggggcggggatcgaaagttacgtcacaa

SEQ ID NO: 111 - DNA, 202 bp, Artificial Sequence
  Synthetic-1/82
aattgcgcaaaatcgattcacgtgtttcgaaattgcgcaaaatcgattttgcgcaatttcgatccccgccccatcgattttgcgcaatttcgaaagttacgtcacaatcgaaatgacgtcaaatcgaaattgcgcaaaatcgatgggactttccatcgaaatgacgtcaaatcgaaatgacgtcaaatcgatccccgcccca

SEQ ID NO: 112 - DNA, 126 bp, Artificial Sequence
  Synthetic-1/83
aagttacgtcacaatcgaaatgacgtcaaatcgaaagttacgtcacaatcgatgggactttccatcgattttgcgcaatttcgatccccgccccatcgatgggactttccatcgatccccgcccca

SEQ ID NO: 113 - DNA, 169 bp, Artificial Sequence
  Synthetic-1/84
aattgcgcaaaatcgaaatgacgtcaaatcgaaattgcgcaaaatcgaaagttacgtcacaatcgatccccgccccatcgatggggcggggatcgattcacgtgtttcgattttgcgcaatttcgatttgacgtcatttcgatggggcggggatcgatttgacgtcatt

SEQ ID NO: 114 - DNA, 201 bp, Artificial Sequence
  Synthetic-1/85
aatgacgtcaaatcgaaacacgtgaatcgattcacgtgtttcgaaacacgtgaatcgattttgcgcaatttcgatggggcggggatcgattgtgacgtaactttcgatggggcggggatcgaaagttacgtcacaatcgattgtgacgtaactttcgaaagttacgtcacaatcgaaacacgtgaatcgatggggcgggga

SEQ ID NO: 115 - DNA, 187 bp, Artificial Sequence
  Synthetic-1/86
tccccgccccatcgattcacgtgtttcgatgggactttccatcgattgtgacgtaactttcgaaacacgtgaatcgaaattgcgcaaaatcgattgtgacgtaactttcgaaatgacgtcaaatcgattcacgtgtttcgaaagttacgtcacaatcgatttgacgtcatttcgaaatgacgtcaaa

SEQ ID NO: 116 - DNA, 171 bp, Artificial Sequence
  Synthetic-1/87
aagttacgtcacaatcgatttgacgtcatttcgaaattgcgcaaaatcgatggggcggggatcgattgtgacgtaactttcgatggggcggggatcgatggggcggggatcgaaacacgtgaatcgaaagttacgtcacaatcgaaatgacgtcaaatcgaaacacgtgaa

SEQ ID NO: 117 - DNA, 171 bp, Artificial Sequence
  Synthetic-1/88
aatgacgtcaaatcgatggaaagtcccatcgatggggcggggatcgattcacgtgtttcgaaatgacgtcaaatcgatggggcggggatcgatttgacgtcatttcgatggaaagtcccatcgatccccgccccatcgattgtgacgtaactttcgattgtgacgtaactt

SEQ ID NO: 118 - DNA, 106 bp, Artificial Sequence
  Synthetic-1/89
aatgacgtcaaatcgatggggcggggatcgatggggcggggatcgaaattgcgcaaaatcgatttgacgtcatttcgatttgacgtcatttcgaaattgcgcaaaa

SEQ ID NO: 119 - DNA, 175 bp, Artificial Sequence
  Synthetic-1/90
ttgtgacgtaactttcgaaatgacgtcaaatcgatccccgccccatcgaaattgcgcaaaatcgattgtgacgtaactttcgattcacgtgtttcgaaagttacgtcacaatcgatccccgccccatcgattgtgacgtaactttcgatggggcggggatcgatttgacgtcatt

SEQ ID NO: 120 - DNA, 252 bp, Artificial Sequence
  Synthetic-1/91
ttcacgtgtttcgaaattgcgcaaaatcgatttgacgtcatttcgaaacacgtgaatcgaaagttacgtcacaatcgaaagttacgtcacaatcgattgtgacgtaactttcgattttgcgcaatttcgattttgcgcaatttcgatccccgccccatcgaaattgcgcaaaatcgatttgacgtcatttcgatttgacgtcatttcgattttgcgcaatttcgaaatgacgtcaaatcgatggggcgggga

SEQ ID NO: 121 - DNA, 158 bp, Artificial Sequence
  Synthetic-1/92
aatgacgtcaaatcgattcacgtgtttcgattgtgacgtaactttcgatttgacgtcatttcgaaagttacgtcacaatcgaaagttacgtcacaatcgaaacacgtgaatcgaaagttacgtcacaatcgaaacacgtgaatcgattttgcgcaatt

SEQ ID NO: 122 - DNA, 157 bp, Artificial Sequence
  Synthetic-1/93
ttttgcgcaatttcgaaagttacgtcacaatcgatggggcggggatcgaaacacgtgaatcgatccccgccccatcgatggggcggggatcgatgggactttccatcgattgtgacgtaactttcgatttgacgtcatttcgattgtgacgtaactt

SEQ ID NO: 123 - DNA, 208 bp, Artificial Sequence
  Synthetic-1/94
aattgcgcaaaatcgaaacacgtgaatcgatggggcggggatcgatggaaagtcccatcgatgggactttccatcgattgtgacgtaactttcgatttgacgtcatttcgatggggcggggatcgattgtgacgtaactttcgatgggactttccatcgattgtgacgtaactttcgaaatgacgtcaaatcgattgtgacgtaactt

SEQ ID NO: 124 - DNA, 156 bp, Artificial Sequence
  Synthetic-1/95
tccccgccccatcgatccccgccccatcgatggaaagtcccatcgaaagttacgtcacaatcgaaagttacgtcacaatcgattcacgtgtttcgaaatgacgtcaaatcgaaattgcgcaaaatcgatttgacgtcatttcgaaatgacgtcaaa

SEQ ID NO: 125 - DNA, 201 bp, Artificial Sequence
  Synthetic-1/96
aacacgtgaatcgattttgcgcaatttcgattcacgtgtttcgaaattgcgcaaaatcgaaacacgtgaatcgaaacacgtgaatcgaaattgcgcaaaatcgattgtgacgtaactttcgatggggcggggatcgaaatgacgtcaaatcgattgtgacgtaactttcgattgtgacgtaactttcgattttgcgcaatt

SEQ ID NO: 126 - DNA, 205 bp, Artificial Sequence
  Synthetic-2/01
tgggactttccatcgatggggcggggatcgaaacacgtgaatcgattcacgtgtttcgatgggactttccatcgattcacgtgtttcgattcacgtgtttcgatgggactttccatcgatgggactttccatcgatgggactttccatcgattcacgtgtttcgaaacacgtgaatcgatgggactttccatcgattcacgtgtt

SEQ ID NO: 127 - DNA, 226 bp, Artificial Sequence
  Synthetic-2/02
aacacgtgaatcgatggaaagtcccatcgatggaaagtcccatcgatggaaagtcccatcgaaacacgtgaatcgaaacacgtgaatcgattcacgtgtttcgatggaaagtcccatcgattcacgtgtttcgaaattgcgcaaaatcgatggaaagtcccatcgattcacgtgtttcgatgggactttccatcgatgggactttccatcgattcacgtgtttcga

SEQ ID NO: 128 - DNA, 209 bp, Artificial Sequence
  Synthetic-2/03
aacacgtgaatcgaaacacgtgaatcgatggaaagtcccatcgatccccgccccatcgaaacacgtgaatcgatggaaagtcccatcgattcacgtgtttcgatggaaagtcccatcgatggaaagtcccatcgattcacgtgtttcgatggaaagtcccatcgatggaaagtcccatcgatgggactttccatcgattttgcgcaatt

SEQ ID NO: 129 - DNA, 134 bp, Artificial Sequence
  Synthetic-2/04
tgggactttccatcgaaacacgtgaatcgatgggactttccatcgaaacacgtgaatcgatgggactttccatcgatggaaagtcccatcgatggaaagtcccatcgaaacacgtgaatcgatgggactttcca

SEQ ID NO: 130 - DNA, 197 bp, Artificial Sequence
  Synthetic-2/05
tggaaagtcccatcgatgggactttccatcgatggggcggggatcgatgggactttccatcgaaattgcgcaaaatcgattcacgtgtttcgattcacgtgtttcgatgggactttccatcgattttgcgcaatttcgatgggactttccatcgattcacgtgtttcgatgggactttccatcgatggaaagtccca

SEQ ID NO: 131 - DNA, 165 bp, Artificial Sequence
  Synthetic-2/06
tggaaagtcccatcgatggaaagtcccatcgatgggactttccatcgatgggactttccatcgattcacgtgtttcgatgggactttccatcgatccccgccccatcgattcacgtgtttcgatggaaagtcccatcgaaacacgtgaatcgaaattgcgcaaaa

SEQ ID NO: 132 - DNA, 153 bp, Artificial Sequence
  Synthetic-2/07
tggaaagtcccatcgatggaaagtcccatcgattcacgtgtttcgatgggactttccatcgatccccgccccatcgatggaaagtcccatcgatggaaagtcccatcgatggaaagtcccatcgattttgcgcaatttcgatgggactttcca

SEQ ID NO: 133 - DNA, 165 bp, Artificial Sequence
  Synthetic-2/08
ttcacgtgtttcgattcacgtgtttcgatggaaagtcccatcgaaattgcgcaaaatcgatggaaagtcccatcgatggaaagtcccatcgatccccgccccatcgatgggactttccatcgatgggactttccatcgatggaaagtcccatcgaaacacgtgaa

SEQ ID NO: 134 - DNA, 179 bp, Artificial Sequence
  Synthetic-2/09
tggaaagtcccatcgatggaaagtcccatcgaaacacgtgaatcgatgggactttccatcgattttgcgcaatttcgatgggactttccatcgatggaaagtcccatcgattcacgtgtttcgatgggactttccatcgatggggcggggatcgattcacgtgtttcgaaacacgtgaa

SEQ ID NO: 135 - DNA, 231 bp, Artificial Sequence
  Synthetic-2/10
ttcacgtgtttcgatgggactttccatcgaaattgcgcaaaatcgatggaaagtcccatcgatgggactttccatcgattcacgtgtttcgatgggactttccatcgattttgcgcaatttcgatgggactttccatcgatgggactttccatcgatggaaagtcccatcgatccccgccccatcgatgggactttccatcgatgggactttccatcgatggaaagtccca

SEQ ID NO: 136 - DNA, 304 bp, Artificial Sequence
  Synthetic-2/11
aacacgtgaatcgatggaaagtcccatcgaaacacgtgaatcgatgggactttccatcgatggaaagtcccatcgatggaaagtcccatcgatggaaagtcccatcgatgggactttccatcgattcacgtgtttcgatggaaagtcccatcgatggaaagtcccatcgatggaaagtcccatcgatggaaagtcccatcgattttgcgcaatttcgatggaaagtcccatcgattcacgtgtttcgatgggactttccatcgattcacgtgtttcgatgggactttccatcgattcacgtgtt

SEQ ID NO: 137 - DNA, 166 bp, Artificial Sequence
  Synthetic-2/12
tggaaagtcccatcgattcacgtgtttcgatgggactttccatcgatgggactttccatcgaaattgcgcaaaatcgatggaaagtcccatcgatgggactttccatcgatgggactttccatcgattcacgtgtttcgattttgcgcaatttcgaaacacgtgaa

SEQ ID NO: 138 - DNA, 286 bp, Artificial Sequence
  Synthetic-2/13
aacacgtgaatcgattttgcgcaatttcgaaattgcgcaaaatcgaaacacgtgaatcgaaattgcgcaaaatcgatgggactttccatcgatgggactttccatcgattcacgtgtttcgatgggactttccatcgatggaaagtcccatcgaaacacgtgaatcgaaacacgtgaatcgatccccgccccatcgatgggactttccatcgatggggcggggatcgatgggactttccatcgaaacacgtgaatcgattttgcgcaatttcgatgggactttcca

SEQ ID NO: 139 - DNA, 183 bp, Artificial Sequence
  Synthetic-2/14
aacacgtgaatcgatggaaagtcccatcgatggaaagtcccatcgatgggactttccatcgatggggcggggatcgatgggactttccatcgatggaaagtcccatcgatccccgccccatcgattttgcgcaatttcgatggaaagtcccatcgatggggcggggatcgatgggactttcca

SEQ ID NO: 140 - DNA, 135 bp, Artificial Sequence
  Synthetic-2/15
tgggactttccatcgatccccgccccatcgattcacgtgtttcgatggaaagtcccatcgatggaaagtcccatcgatgggactttccatcgatggaaagtcccatcgattcacgtgtttcgatgggactttcca

SEQ ID NO: 141 - DNA, 151 bp, Artificial Sequence
  Synthetic-2/16
tgggactttccatcgatccccgccccatcgaaacacgtgaatcgatgggactttccatcgatggaaagtcccatcgaaattgcgcaaaatcgatgggactttccatcgatgggactttccatcgatccccgccccatcgatggggcgggga

SEQ ID NO: 142 - DNA, 317 bp, Artificial Sequence
  Synthetic-2/17
ttcacgtgtttcgatgggactttccatcgatccccgccccatcgatccccgccccatcgattcacgtgtttcgaaacacgtgaatcgatgggactttccatcgatgggactttccatcgatgggactttccatcgattcacgtgtttcgatgggactttccatcgatggaaagtcccatcgatccccgccccatcgatggaaagtcccatcgaaattgcgcaaaatcgatgggactttccatcgaaacacgtgaatcgattcacgtgtttcgatgggactttccatcgatgggactttccatcgatgggactttcca

SEQ ID NO: 143 - DNA, 182 bp, Artificial Sequence
  Synthetic-2/18
tggaaagtcccatcgatggaaagtcccatcgatgggactttccatcgatggaaagtcccatcgatccccgccccatcgatggaaagtcccatcgatgggactttccatcgatccccgccccatcgattcacgtgtttcgatgggactttccatcgattttgcgcaatttcgaaacacgtgaa

SEQ ID NO: 144 - DNA, 211 bp, Artificial Sequence
  Synthetic-2/19
ttcacgtgtttcgatggaaagtcccatcgatgggactttccatcgaaattgcgcaaaatcgatgggactttccatcgatgggactttccatcgattcacgtgtttcgatggggcggggatcgatgggactttccatcgatgggactttccatcgaaacacgtgaatcgaaattgcgcaaaatcgatgggactttccatcgaaacacgtgaa

SEQ ID NO: 145 - DNA, 188 bp, Artificial Sequence
  Synthetic-2/20
aacacgtgaatcgaaacacgtgaatcgatggaaagtcccatcgattcacgtgtttcgattcacgtgtttcgaaacacgtgaatcgatggaaagtcccatcgatccccgccccatcgattcacgtgtttcgatgggactttccatcgattcacgtgtttcgatgggactttccatcgatccccgcccca

SEQ ID NO: 146 - DNA, 151 bp, Artificial Sequence
  Synthetic-2/21
tgggactttccatcgatgggactttccatcgattcacgtgtttcgaaacacgtgaatcgatggaaagtcccatcgatgggactttccatcgatggggcggggatcgattcacgtgtttcgatggaaagtcccatcgattcacgtgtttcga

SEQ ID NO: 147 - DNA, 147 bp, Artificial Sequence
  Synthetic-2/22
tgggactttccatcgattcacgtgtttcgatccccgccccatcgaaacacgtgaatcgatggaaagtcccatcgattttgcgcaatttcgattcacgtgtttcgatggaaagtcccatcgaaacacgtgaatcgatgggactttcca

SEQ ID NO: 148 - DNA, 150 bp, Artificial Sequence
  Synthetic-2/23
tggaaagtcccatcgattcacgtgtttcgatggaaagtcccatcgatgggactttccatcgatggggcggggatcgatggaaagtcccatcgatgggactttccatcgatgggactttccatcgatggggcggggatcgattcacgtgtt

SEQ ID NO: 149 - DNA, 196 bp, Artificial Sequence
  Synthetic-2/24
aattgcgcaaaatcgaaacacgtgaatcgatggaaagtcccatcgatggaaagtcccatcgaaacacgtgaatcgatggaaagtcccatcgatccccgccccatcgatgggactttccatcgattcacgtgtttcgatggggcggggatcgatgggactttccatcgaaattgcgcaaaatcgatgggactttcca

SEQ ID NO: 150 - DNA, 155 bp, Artificial Sequence
  Synthetic-2/25
tgggactttccatcgatccccgccccatcgatgggactttccatcgatgggactttccatcgatgggactttccatcgatggaaagtcccatcgatggaaagtcccatcgattttgcgcaatttcgatggaaagtcccatcgatgggactttcca

SEQ ID NO: 151 - DNA, 162 bp, Artificial Sequence
  Synthetic-2/26
ttttgcgcaatttcgattcacgtgtttcgatccccgccccatcgatgggactttccatcgatccccgccccatcgatggaaagtcccatcgaaacacgtgaatcgatggggcggggatcgattcacgtgtttcgatggaaagtcccatcgatccccgcccca

SEQ ID NO: 152 - DNA, 165 bp, Artificial Sequence
  Synthetic-2/27
tggaaagtcccatcgatgggactttccatcgattcacgtgtttcgatggaaagtcccatcgatggaaagtcccatcgaaacacgtgaatcgaaacacgtgaatcgaaattgcgcaaaatcgaaacacgtgaatcgatccccgccccatcgattcacgtgtttcga

SEQ ID NO: 153 - DNA, 122 bp, Artificial Sequence
  Synthetic-2/28
aacacgtgaatcgatggaaagtcccatcgatggaaagtcccatcgatgggactttccatcgatggaaagtcccatcgatgggactttccatcgatggaaagtcccatcgattttgcgcaatt

SEQ ID NO: 154 - DNA, 184 bp, Artificial Sequence
  Synthetic-2/29
aattgcgcaaaatcgatggaaagtcccatcgatccccgccccatcgatccccgccccatcgattcacgtgtttcgatggaaagtcccatcgattcacgtgtttcgatgggactttccatcgaaacacgtgaatcgatggaaagtcccatcgattttgcgcaatttcgatgggactttccatcga

SEQ ID NO: 155 - DNA, 136 bp, Artificial Sequence
  Synthetic-2/30
aacacgtgaatcgatgggactttccatcgatggaaagtcccatcgatgggactttccatcgaaacacgtgaatcgatgggactttccatcgattttgcgcaatttcgatggaaagtcccatcgatggaaagtccca

SEQ ID NO: 156 - DNA, 163 bp, Artificial Sequence
  Synthetic-2/31
ttcacgtgtttcgatccccgccccatcgatccccgccccatcgatggaaagtcccatcgatccccgccccatcgaaacacgtgaatcgattttgcgcaatttcgatggaaagtcccatcgaaacacgtgaatcgatggaaagtcccatcgatgggactttcca

SEQ ID NO: 157 - DNA, 146 bp, Artificial Sequence
  Synthetic-2/32
tgggactttccatcgatgggactttccatcgatccccgccccatcgaaacacgtgaatcgattcacgtgtttcgatgggactttccatcgatggaaagtcccatcgatccccgccccatcgaaacacgtgaatcgaaacacgtgaa

SEQ ID NO: 158 - DNA, 137 bp, Artificial Sequence
  Synthetic-2/33
tggaaagtcccatcgaaacacgtgaatcgatggggcggggatcgatgggactttccatcgatggaaagtcccatcgaaacacgtgaatcgattcacgtgtttcgaaattgcgcaaaatcgatggaaagtcccatcga

SEQ ID NO: 159 - DNA, 118 bp, Artificial Sequence
  Synthetic-2/34
ttttgcgcaatttcgaaacacgtgaatcgatccccgccccatcgatggaaagtcccatcgatgggactttccatcgatgggactttccatcgatggggcggggatcgaaacacgtgaa

SEQ ID NO: 160 - DNA, 170 bp, Artificial Sequence
  Synthetic-2/35
tggaaagtcccatcgatggaaagtcccatcgatggggcggggatcgattttgcgcaatttcgatccccgccccatcgaaattgcgcaaaatcgatgggactttccatcgatggaaagtcccatcgatgggactttccatcgatggaaagtcccatcgaaattgcgcaaaa

SEQ ID NO: 161 - DNA, 134 bp, Artificial Sequence
  Synthetic-2/36
tggaaagtcccatcgaaacacgtgaatcgatggaaagtcccatcgatggggcggggatcgatgggactttccatcgatgggactttccatcgaaattgcgcaaaatcgaaacacgtgaatcgatggggcgggga

SEQ ID NO: 162 - DNA, 133 bp, Artificial Sequence
  Synthetic-2/37
tccccgccccatcgatgggactttccatcgatgggactttccatcgatggaaagtcccatcgatgggactttccatcgaaacacgtgaatcgaaacacgtgaatcgatccccgccccatcgatccccgcccca

SEQ ID NO: 163 - DNA, 117 bp, Artificial Sequence
  Synthetic-2/38
tggggcggggatcgaaacacgtgaatcgattttgcgcaatttcgatgggactttccatcgatgggactttccatcgattcacgtgtttcgattcacgtgtttcgatggaaagtccca

SEQ ID NO: 164 - DNA, 103 bp, Artificial Sequence
  Synthetic-2/39
tgggactttccatcgatgggactttccatcgatggggcggggatcgattcacgtgtttcgattttgcgcaatttcgattcacgtgtttcgatgggactttcca

SEQ ID NO: 165 - DNA, 135 bp, Artificial Sequence
  Synthetic-2/40
tccccgccccatcgatggaaagtcccatcgatgggactttccatcgattcacgtgtttcgatggggcggggatcgatggaaagtcccatcgatggaaagtcccatcgattttgcgcaatttcgatggggcgggga

SEQ ID NO: 166 - DNA, 132 bp, Artificial Sequence
  Synthetic-2/41
aacacgtgaatcgaaattgcgcaaaatcgattcacgtgtttcgaaacacgtgaatcgaaattgcgcaaaatcgatggaaagtcccatcgatggaaagtcccatcgattcacgtgtttcgaaattgcgcaaaa

SEQ ID NO: 167 - DNA, 120 bp, Artificial Sequence
  Synthetic-2/42
tggaaagtcccatcgatggggcggggatcgatggaaagtcccatcgatgggactttccatcgaaacacgtgaatcgatggggcggggatcgaaattgcgcaaaatcgaaattgcgcaaaa

SEQ ID NO: 168 - DNA, 135 bp, Artificial Sequence
  Synthetic-2/43
tggggcggggatcgattcacgtgtttcgaaattgcgcaaaatcgatgggactttccatcgatccccgccccatcgatgggactttccatcgatggggcggggatcgaaattgcgcaaaatcgatgggactttcca

SEQ ID NO: 169 - DNA, 146 bp, Artificial Sequence
  Synthetic-2/44
ttttgcgcaatttcgaaacacgtgaatcgattcacgtgtttcgaaacacgtgaatcgattttgcgcaatttcgattcacgtgtttcgaaattgcgcaaaatcgattttgcgcaatttcgattcacgtgtttcgatgggactttcca

SEQ ID NO: 170 - DNA, 84 bp, Artificial
  Synthetic-CMV core promoter
aggtctatataagcagagctcgtttagtgaaccgtcagatcgcctagatacgccatccacgctgttttgacctccatagaagac

SEQ ID NO: 171 - DNA, 16 bp, Artificial
  Synthetic-NFkB-RE
tcgatgggactttcca

SEQ ID NO: 172 - DNA, 16 bp, Artificial
  Synthetic-NFkB-RE
tcgatggaaagtccca

(19) United States
(12) Patent Application Publication
(10) Pub. No.: US 2017/0002378 A1
(43) Pub. Date: Jan. 5, 2017

(54) Synthetic Promoters

(71) Applicant: UCB BIOPHARMA SPRL, Brussels (BE)

(72) Inventors: David Cameron James, Slough, Berkshire (GB); Adam John Brown, Slough, Berkshire (GB)

(21) Appl. No.: 15/100,071

(22) PCT Filed: Nov. 28, 2014
(86) PCT No.: PCT/EP2014/076024

§ 371 Date: May 27, 2016

(30) Foreign Application Priority Data
Nov. 29, 2013 (GB) 1321109.9
Jul. 1, 2014 (GB) 1411717.0

(51) Int. Cl. C12N 15/85; C12N 15/10

(52) U.S. Cl. CPC C12N 15/85; C12N 15/1058; C12N 2830/008

(57) ABSTRACT

CHO cell-specific synthetic promoter constructs for expressing recombinant proteins, a library of promoter constructs thereof, and a method for producing the promoter constructs. The promoter constructs enable precise control of recombinant gene transcription over three orders of magnitude, with the top expressing promoters capable of double the transcriptional activity of the CMV promoter.

The present invention relates to synthetic promoter constructs for use in mammalian cells, in particular Chinese hamster ovary (CHO) cells and recombinant host cells comprising the same. The invention further relates to a method for producing these promoter constructs, said recombinant cells, and use of any one of the same, for example in the expression of recombinant proteins.

### BACKGROUND

Chinese hamster ovary (CHO) cells are derived from the ovary of the Chinese hamster and are widely used in genetic research; toxicity screening and gene expression, particularly for the expression of recombinant proteins. They were first introduced in the 1960s, are grown in suspension culture and require proline in their culture medium. CHO cells are the most commonly used mammalian host cell line for the large scale production of recombinant protein therapeutics. Thus, CHO cells are an important tool in the biopharmaceutical industry.

One of the major challenges faced when using CHO cells for producing recombinant proteins is that it can be difficult to accurately control the levels of expression of the recombinant proteins. The ability to accurately control the expression levels of recombinant proteins is especially important given that the current bioindustrial portfolio of candidate biopharmaceuticals is diversifying to include many “difficult-to-express” (DTE) proteins that require protein-specific control of gene transcription kinetically coordinated with polypeptide-specific folding and assembly rates. For example, it has been shown that monoclonal antibody (mAb) expression can be substantially improved by titration of heavy chain (HC) and light chain (LC) gene expression ratios (Ho et al. 2013).

Development of new cell factories with optimized phenotypes (protein folding, glycosylation, apoptosis, etc.) will also require the ability to simultaneously vary the transcriptional activity of multiple genes (Datta et al. 2013, Lanza et al. 2012).

At the moment, recombinant gene transcription in CHO cells is still routinely controlled with strong viral promoters (such as the human cytomegalovirus immediate early 1 (hCMV-IE1, or CMV) promoter) despite links to cellular stress induction, cell-cycle dependency, and epigenetic silencing (Dale 2006; Kim et al. 2011). CMV is a “gold standard” promoter currently used to drive expression of most biopharmaceutical products. It is a highly complex element evolved by the virus to enable it to infect many mammalian host cells (Stinski and Isomura 2008). However, despite its use for >25 years to drive biopharmaceutical production, little is known about how it functions mechanistically in the CHO cell and therefore strategies to precisely control or improve its transcriptional activity are not generally available.

Synthetic promoters therefore offer a potentially attractive solution, as they can replace functionally ill-defined and uncontrollable genetic elements in expression vectors with sophisticated, bespoke controllers that can engineer host cell function predictably.

Synthetic promoters have been traditionally produced by screening either (i) randomised DNA sequences or synthetic oligonucleotide repeats or (ii) assemblies of known cis-regulatory elements (e.g. transcription factor binding sites, or regulatory elements, or TFREs), both upstream of minimal core promoter motifs. These efforts have resulted in synthetic promoter libraries designed to function in a range of organisms, predominantly microbial, such as Corynebacterium (Yim et al. 2013), Saccharomyces (Blazeck et al. 2012), Pichia (Stadlmayr et al. 2010) and Streptomyces (10), but also in mammalian cells (Ferreira et al. 2011; Schlabach et al. 2010). These studies have demonstrated that the exquisite gene expression control offered by synthetic promoters is highly context-dependent, necessitating promoters to be specifically constructed for each host cell type. Schlabach et al., for example describe the synthesis of synthetic promoters for mammalian cells screened in HeLa cells (207% of CMV activity), but also report highly variable reporter gene expression in different mammalian cell types (21-113% of CMV) (Schlabach et al. 2010).

However, few studies have previously explored the utilization of synthetic promoters in CHO cells. Tornøe et al created a small pool of promoters (<20) with a tenfold range in activity by randomising the sequences separating TFREs within a chimeric promoter (Tornoe et al. 2002). Further, Grabherr et al constructed five synthetic promoters with approximately equivalent activities by constructing contiguous sequences with nucleotide compositions mimicking those found in highly active promoters (Grabherr et al. 2011). However, both of these studies targeted broad activity in mammalian cells (tested in multiple diverse cell lines) and neither sought to specifically design synthetic promoters to function in concert with the transactivational machinery of CHO cell factories.

Accordingly, neither of the above promoter constructs enable predictable, precise and robust control of CHO gene expression over a broad dynamic range and synthetic promoter activities reported were significantly below that of CMV.

More recently, Le et at utilized transcriptomics data to identify CHO endogenous promoters with desired expression dynamics (Le et al. 2013). Whilst this is a promising avenue to identify promoters with discrete expression levels, it is limited to naturally occurring activities. Moreover, it can be a significant challenge to define the genomic regulatory sequences controlling expression of specific genes.

Hence, there is a need for Chinese hamster ovary cell (CHO)-specific gene expression control technology that could offer tractable solutions to the present biopharmaceutical production challenges. The present invention seeks to address the above challenges.

### SUMMARY OF INVENTION

The inventors functionally screened motifs associated with transcription factor regulatory elements and for the first time identified the elements that are active in CHO cells.

The active elements have been used and combined to prepare synthetic promoters useful in recombinant host cells, in particular CHO cells.

These CHO-active elements were then employed to construct large synthetic promoter libraries exhibiting expression over three orders of magnitude.

The present inventors have thus constructed the first libraries of synthetic promoters designed specifically to harness the pre-existing transcriptional activation machinery of CHO cell factories.

The inventors have further shown that next-generation libraries can be tailored to specific expression levels and constructed multiple promoters with activity exceeding hCMV-IE1 in, for example transient production processes. Moreover, relative promoter activities across a broad dynamic expression range were maintained in both divergent CHO cell hosts and long-term fed-batch transient production.

The robust precision gene expression control enabled by this synthetic promoter technology will facilitate creation of bespoke, synthetic mammalian cell factories harbouring multiple genetic components operating at an optimal, designed stoichiometry. Further, it can be utilized to enable optimized, protein-specific control of recombinant gene transcription to facilitate the production of difficult to express proteins. Synthetic promoters of the present invention that allow higher titers than CMV in fed-batch transient SEAP production could also be utilized to optimize the production (for example transient expression) of early stage products, for example, development material for toxicology and clinical trials testing (Daramola et al. 2013).

In a first aspect, there is provided a CHO cell comprising a synthetic promoter suitable for eliciting recombinant protein expression therein said synthetic promoter comprising a promoter core and upstream thereof two or more transcription factor regulatory elements independently selected from the group consisting of NFκB-RE, E-box, AP1, CRE, GC-Box, E41F, C/EBPα-RE, OCT and RARE.

Also provide is a synthetic promoter suitable for promoting recombinant protein expression in a CHO cell said synthetic promoter comprising a promoter core and upstream thereof a mixture of two or more transcription factor regulatory elements independently selected from the group consisting of NFκB-RE, E-box, AP1, CRE, GC-Box, E4F1, C/EBPα-RE, OCT and RARE, in particular 2 or more copies of at least one regulatory element.

A method of generating a synthetic promoter suitable for promoting recombinant protein expression in a given mammalian recombinant host cell comprising the steps of:
A) selecting two or more transcription factor regulatory elements suitable for use in the chosen mammalian recombinant host cell line,
B) preparing one or more synthetic promoter constructs comprising two or more of those transcription factor regulatory elements independently selected from those selected in step A),
C) testing the synthetic promoter construct or constructs prepared in step B) for activity in the chosen mammalian recombinant host cell.

The present inventors have also identified at least one transcription factor/transcription factor regulatory element which can negatively regulate gene expression in CHO cells, namely YY1. Thus in one embodiment there is provided a CHO cell wherein the activity of the transcription factor YY1 is knocked down or knocked out.

In one embodiment there is provided use of a block decoy specific to YY1.

The invention is summarised in the following paragraphs:
1. A CHO cell, comprising a synthetic promoter suitable for eliciting recombinant protein expression therein, said synthetic promoter comprising a promoter core and upstream thereof two or more transcription factor regulatory elements independently selected from the group consisting of NFκB-RE, E-box, AP1, CRE, GC-Box, E41F, C/EBPα-RE, OCT and RARE.
2. A CHO cell according to paragraph 1, wherein the promoter core is selected from CMV, SV40, UbC, EF1A, PGK and CAGG, such as a CMV core, in particular hCMV-IE1.
3. A CHO cell according to paragraph 1 or 2, wherein the synthetic promoter comprises 2 to 50 transcription factor regulatory elements, for example 2, 3, 4, 5, 6, 7, 8, 9, 10, 11, 12, 13, 14, 15, 16, 17, 18, 19 or 20, for example 5, 6, 7 or 8 of said regulatory elements.
4. A CHO cell according to any one of paragraphs 1 to 3, wherein the transcription factor regulatory elements are all the same as each other.
5. A CHO cell according to any one of paragraph 1 to 3, wherein the transcription factor regulatory elements are a combination of different types.
6. A CHO cell according to any one of paragraph 1 to 5, wherein the transcription factor regulatory elements are in tandem with each other.
7. A CHO cell according to any one of paragraph 1 to 6, wherein the synthetic promoter comprises NFκB-RE, for example two or more copies thereof.
8. A CHO cell according to any one of paragraph 1 to 7, wherein the synthetic promoter comprises E-box, for example two or more copies thereof
9. A CHO cell according to any one of paragraph 1 to 8, wherein the synthetic promoter comprises CRE, for example two or more copies thereof.
10. A CHO cell according to paragraph 5, wherein each transcription factor regulatory element in the combination is independently selected from NFκB-RE, E-box, GC-Box, C/EBPα-RE, CRE and E41F, in particular selected from NFκB-RE, E-box, GC-Box and C/EBPα-RE, especially NFκB-RE, and E-box, in particular 2 or more copies of at least one regulator element in the combination.
11. A CHO cell according to any one of paragraph 1 to 10, wherein synthetic promoter DNA sequence is 0.9 or less of the amount of full length CMV promoter sequence, for example 0.8, 0.75, 0.7, 0.6, 0.5 or less.
12. A CHO cell according to any one of paragraph 1 to 11, wherein the synthetic promoter has a transcriptional activity per unit DNA sequence thereof which in greater than transcriptional activity per unit DNA of CMV promoter, for example 1.2, 1.4, 1.5, 1.6, 1.8, 2.0, 2.2, 2.4 or 2.5 fold increase in activity.
13. A CHO cell according to any one of paragraph 1 to 12, wherein the CHO cell is selected from CHO-S, CHO-K1 and CHO-DG44.
14. A CHO cell according to any one of paragraph 1 to 13, wherein the activity of the transcription factor regulatory element YY1 is inhibited, for example by a block decoy specific to YY1.
15. A CHO cell according to paragraph 14, wherein the cell is mutated to delete the transcription factor YY1 and/or render it silent thereby inhibiting the activity of YY1.
16. A CHO cell according to any one of paragraph 1 to 15, wherein the cell further comprises a polynucleotide sequence encoding a recombinant protein under the control of the synthetic promoter.
17. A CHO cell according to paragraph 16, wherein the recombinant protein is an antibody or antigen binding fragment thereof
18. A CHO cell according to any one of paragraph 1 to 17, wherein the promoter exhibits improved protein expression in comparison to the promoter core or the wild type promoter.
19. A CHO cell according to paragraph 18, wherein the improved protein expression is a greater level of recombinant protein expression.
20. A CHO cell according to any one of paragraph 1 to 19, wherein the synthetic promoter comprises a sequence shown in any one of SEQ ID NOs: 30 to 169.
21. A CHO cell according to paragraph 20, wherein the synthetic promoter comprises a sequence shown in any one of SEQ ID NOs: 126 to 169.
22. A CHO cell according to any one of the preceding paragraphs, wherein the synthetic promoter does not comprise any CpG islands.
23. A CHO cell, according to any one of paragraphs 1 to 22 comprising two or more promoters, for example two or more synthetic promoters as defined in any one of claims 1 to 22.
24. A CHO cell according to any one of paragraphs 1 to 23, wherein the synthetic promoter has properties suited to the specific cell, for example the transcription factor regulatory elements in the synthetic promoter correspond to the transcription factors found in the cell.
25. A CHO cell according to any one of paragraphs 1 to 23, wherein the synthetic promoter has properties suited to the expression of the specific recombinant protein it is associated with.
26. A synthetic promoter suitable for promoting recombinant protein expression in a CHO cell said synthetic promoter comprising a promoter core and upstream thereof a mixture of two or more transcription factor regulatory elements independently selected from the group consisting of NFκB-RE, E-box, AP1, CRE, GC-Box, E41F, C/EBPα-RE, OCT and RARE, in particular 2 or more copies of at least one regulator element in the combination.
27. A synthetic promoter suitable for promoting recombinant protein expression in a CHO cell said synthetic promoter comprising a promoter core and upstream thereof a mixture of two or more transcription factor regulatory elements independently selected from the group comprising or consisting of NFκB-RE and CRE.
28. A synthetic promoter sequence according to paragraph 26 or 27, wherein synthetic promoter DNA sequence is 0.9 or less of the amount of full length CMV promoter sequence, for example 0.8, 0.75, 0.7, 0.6, 0.5 or less.
29. A synthetic promoter sequence according to any one of paragraph 26 to 28, wherein the synthetic promoter has a transcriptional activity per unit DNA sequence thereof which in greater than transcriptional activity per unit DNA of CMV promoter, for example 1.2, 1.4, 1.5, 1.6, 1.8, 2.0, 2.2, 2.4 or 2.5 fold increase in activity.
30. A method of generating a synthetic promoter suitable for promoting recombinant protein expression in a given mammalian recombinant host cell comprising the steps of:
   a) identifying motifs of transcription factor regulatory elements,
   b) testing each transcription factor regulatory element identified in step a) combined with a promoter core for activity in a chosen mammalian recombinant host cell line,
   c) selecting two or more transcription factor regulatory elements from step (b) which are more active in the chosen mammalian recombinant host cell line than the promoter core alone,
   d) preparing one or more synthetic promoter constructs comprising two or more of those transcription factor regulatory elements independently selected from those selected in step c),
   e) testing the synthetic promoter construct or constructs prepared in step d) for activity in the chosen mammalian recombinant host cell,
   f) identifying the synthetic promoter construct or constructs that exhibit the same or improved protein expression compared to a wild type promoter.
31. A method according to paragraph 30 comprising the further steps:
   g) selecting two or more of those transcription factor regulatory elements which are associated with the constructs identified in step f), and
   h) preparing one or more synthetic promoter constructs comprising a TFRE construct comprising or consisting of those elements independently selected in step g).
32. The method according to paragraph 31, wherein the TFRE constructs prepared in step h comprise transcription factor regulatory elements at a stoichiometry which reflects their relative abundance in the constructs identified in step f).
33. The method according to any one of paragraphs 30 to 32, wherein step (f) of the method further comprises identifying the transcription factor regulatory element or elements that are most frequently associated with promoter constructs which exhibit reduced protein expression compared to the wild type promoter and excluding these in step (g).
34. A method of identifying a synthetic promoter suitable for promoting recombinant protein expression in a given mammalian recombinant host cell at a desired level comprising the steps of:
   a) obtaining two or more synthetic promoter constructs defined in claims 26 to 28.
   b) testing the synthetic promoter constructs obtained in step a) to determine the level of recombinant protein expression driven by each construct in the chosen mammalian recombinant host cell
   c) selecting a synthetic promoter construct tested in step (b) if it promotes recombinant protein expression at the desired level.
35. A method according to paragraph 34, wherein the two or more synthetic promoters obtained in step (a) each comprises a sequence independently selected from any one of SEQ ID NOs: 30 to 169 or SEQ ID NOs 126-169.
36. The method according to paragraph 34 or 35, wherein the desired level of protein expression is higher than that achieved using a wild type promoter.
37. The method according to paragraph 34 or 35, wherein the desired level of protein expression is lower than that achieved using a wild type promoter.
38. A method of constructing a transcription factor regulatory element construct library comprising the step of randomly ligating the transcription factor regulatory elements NFκB-RE and E-box at a ratio of 5:3.
39. A method of constructing a transcription factor regulatory element construct library comprising the step of randomly ligating the transcription factor regulatory elements NFκB-RE, E-box, GC-Box and C/EBPα-RE at a ratio of 5:3:1:1.
40. A CHO cell wherein the activity of the transcription factor regulator element YY1 activity is knocked down or knocked out.
41. A CHO cell according to paragraph 40, wherein the YY1 activity is knocked down or knocked out by a block decoy specific to YY1.
42. A CHO cell according to paragraph 40, wherein the cell is mutated to delete the transcription factor regulatory element YY1 and/or render it silent thereby inhibiting the activity of YY1.
43. A CHO cell according to any one of paragraphs 40 to 43, wherein the cell further comprises a polynucleotide sequence encoding a recombinant protein under the control of the synthetic promoter.
44. A CHO cell according to paragraph 43, wherein the recombinant protein is an antibody or antigen binding fragment thereof
45. A CHO cell according to any one of paragraphs 40 to 44, wherein the cell type is selected from CHO-S, CHO-K1 and CHO-DG44.

Advantageously, the inventors found that certain transcription factor regulatory elements are particularly active in CHO cells and that when incorporated into a promoter construct of the present invention are able to regulate transcription, for example increase the level of expression of the recombinant protein compared to when the core promoter alone is used. Accordingly, the inventors have found that promoter constructs which comprise combinations of these transcription factor regulatory elements enable precise control of recombinant gene transcription over a broad dynamic range in CHO cells (over three orders of magnitude).

Advantageously, because the synthetic promoters are specifically designed, rather than evolved, the promoters are devoid of redundant sequences and accordingly have far more efficient transcriptional activity per unit DNA sequence. For example, synthetic promoter 2/01 (SEQ ID NO:126) as described herein exhibits a 2.2-fold increase in activity over the CMV promoter but is less than half the size of CMV.

However, precise control is not always about increased expression because other factors such as the stability of the recombinant system, the requirement for appropriate folding, and the ability to express difficult to express proteins, such as bispecific antibodies, can mean that the highest levels of expression of recombinant protein are not always the ultimate goal. Indeed, a significant advantage of the present invention is the flexibility provided, which enables the level of expression to be adjusted as required depending on the combination of TFREs included in the synthetic promoter.

For example, for easy to express proteins, where transcription rates have been shown to exert a high level of control over production, (McLeod et al. 2011; O'Callaghan et al. 2010), the synthetic promoters of the present invention may be used to maximise recombinant gene transcription levels (for example, by using synthetic promoter 2/01).

In comparison, for difficult to express proteins (e.g. bispecific antibodies, fusion proteins), where maximizing transcription is unlikely to be beneficial, the promoters may be utilized to provide optimized protein-specific transcription activity kinetically coordinated with polypeptide-specific folding and assembly rates. For example, a potential application is in monoclonal antibody (mAb) expression where, for example, two synthetic promoters of varying activity could be used to achieve mAb-specific light chain: heavy chain (LC: HC) expression ratios to optimize mAb production (Ho et al. 2013; Pybus et al. 2013).

Thus in one embodiment the synthetic promoter is adapted or chosen to be suitable for expression of the particular recombinant protein, i.e. the properties of the promoter are matched with suitable expression of the specific protein.

Suitable expression of the particular protein as employed herein refers to a promoter that maximises the expression of the given protein in a desirable form, for example increases yield and/or increases amount of protein which is appropriately folded, when compared to the results obtained for a known CMV promoter.

In one embodiment, the synthetic promoters of the present invention do not contain CpG islands. CpG islands in CMV have been shown to interfere with the functionality of matrix attachment regions (Girod et al. 2005). The methylation of cytosines within CpG islands is known to produce instability. Hence, because the promoters of the present invention do not have CpG islands, the promoters may have enhanced stability, resulting in highly reproducible levels of expression when used to express recombinant proteins. Advantageously, the synthetic promoters of the present invention are more compatible with existing transcription enhancing technologies such as ubiquitously-acting chromatin opening elements, bacterial artificial chromosomes and site-specific integration systems (Mader et al. 2012; Zhou et al. 2010).

Further, the promoters of the present invention may be utilized with other expression control technologies in research applications requiring highly precise regulation. For example, they may be employed in conjunction with TFRE-specific decoy technology (Brown et al. 2013), or recently described synthetic elements that control translation initiation rates (Ferreira et al. 2013). Thus in one aspect there is provided a block decoy comprising transcription factor regulatory elements specific to at least YY1.

In one embodiment the two or more transcription factor regulatory elements in the synthetic promoters of the present disclosure are in tandem.

In one embodiment, the two or more transcription factor regulatory elements in the synthetic promoters of the present disclosure are all the same.

In one embodiment, the two or more transcription factor regulatory elements in the synthetic promoters of the present disclosure are a combination of different types.

In one embodiment, the two or more transcription factor regulatory elements are a combination of different types including multiple copies of one or more of the same TFRE, such as 3, 4, 5, 6, 7, 8, 9, 10 or more copies of one or more of the same TFRE. In one example the synthetic promoter comprises two or more, such as 3, 4, 5, 6, 7, 8, 9, 10 or more copies of NFκB-RE.

In one embodiment the synthetic promoter comprises two or more, such as 3, 4, 5, 6, 7, 8, 9, 10 or more copies of E-box.

In one embodiment the synthetic promoter comprises two or more, such as 3, 4, 5, 6, 7, 8, 9, 10 or more copies of CRE.

In one embodiment the synthetic promoter comprises two or more, such as 3, 4, 5, 6, 7, 8, 9, 10 or more copies of NFκB-RE and two or more, such as 3, 4, 5, 6, 7, 8, 9, 10 or more copies of E-box.

In one embodiment the synthetic promoter comprises two or more, such as 3, 4, 5, 6, 7, 8, 9, 10 or more copies of NFκB-RE and two or more, such as 3, 4, 5, 6, 7, 8, 9, 10 or more copies of CRE.

When the synthetic promoter comprises two or more, such as two, types of transcription factor regulatory elements and multiple copies of one or more then these may be provided in any suitable arrangement, for example as one complete string of a first TFRE (such as 3, 4, 5, 6, 7, 8, 9, 10 or more copies of NFκB-RE) followed by a complete string of at least a second TFRE (such as 3, 4, 5, 6, 7, 8, 9, 10 or more copies of E-box). Alternatively the first and second TFRE etc may be mixed randomly or in a pattern wherein one copy (or a small string) of a first TFRE is followed by one copy (or a small string) of a second TFRE, which in turn is followed by one copy (or a small string) of the first TFRE, etc.

Advantageously, the inventors have found that when different types of transcription factor regulatory elements are combined within a synthetic promoter, it results in different levels of modulation of expression levels, for example increased or decreased expression levels relative to the CMV promoter. Alternatively or additionally the synthetic promoters provided herein may have improved expression stability and/or be optimised for expression of a specific protein, such that the protein is expressed in a desirable form, for example properly folded.

Promoter constructs comprising combinations of transcription factor regulatory elements have a different transcriptional activity compared to when a construct consists of only one type of transcription factory regulatory element and therefore significantly extends the potential range of transcription activities that can be obtained, thereby resulting in a wide range of different transcriptional activities from synthetic promoters that can then be matched to suit expression of a particular recombinant protein. Accordingly, the promoter constructs can be used for precise control of protein expression in mammalian cells, in particular CHO cells.

For example, by utilizing specific TFRE combinations, the synthetic promoters may be refined to exhibit desirable bioproduction functionalities, such as increased activity at sub-physiological temperatures (Al-Fageeh et al. 2006) or during stationary phase cell growth (Prentice et al. 2007).

In one embodiment the synthetic promoters of the present disclosure are smaller than the wild type CMV promoter, for example the sequence is about 0.9 or less of the size of the CMV promoter, such as 0.8, 0.75, 0.7, 0.6, 0.5 or less of the size of the CMV promoter. As employed herein 0.5 of the size refers to a synthetic promoter which is half the size of the CMV promoter i.e. contains about ½ the number of bases pairs of the CMV promoter or is half the molecular weight of the CMV promoter.

Advantageously being able to prepare synthetic promoters which are smaller than the CMV promoter allows more room in expression vectors encoding other genetic material, for example larger recombinant genes or increased numbers of functional genes (e.g. chaperones, redox proteins, unfolded protein response transactivators, vesicle trafficking components, etc.) in multigene engineering vectors. The smaller synthetic promoters also have an increased transcriptional activity per unit DNA than the corresponding value for CMV promoter.

In one embodiment, the two or more transcription factor regulatory elements are independently selected from the group consisting of Activator protein 1 regulatory element (AP1-RE), CC(A/T)₆GG element (CArG), CCAAT displacement protein regulatory element (CDP-RE), CCAAT-enhancer Binding Protein α regulatory element (C/EBPα-RE), Cellular Myeloblastosis regulatory element (cMyb-RE), cAMP regulatory element (CRE), Elongation Factor 2 regulatory element (E2F-RE), E4F1 regulatory element (E4F1-RE), Early Growth Response Protein 1 regulatory element (EGR1-RE), ERR-alpha regulatory element (ERRE), Enhancer Box (E-Box), GATA-1 regulatory element (GATA-RE), GC-box, Glucocorticoid regulatory element (GRE), Growth Factor Independence 1 regulatory element (Gfi1-RE), Helios regulatory element (HRE), Hepatocyte Nuclear Factor 1 regulatory element (HNF-RE), Insulin Promoter Factor 1 regulatory element (IPF1-RE), Interferon-stimulated regulatory element (ISRE), Myocyte enhancer Factor 2 regulatory element (MEF2-RE), Msx Homeobox regulatory element (MSX-RE), Nerve Growth Factor-Induced Gene-B regulatory element (NBRE), Nuclear Factor 1 regulatory element (NF1-RE), Nuclear Factor of Activated T Cells regulatory element (NFAT-RE), Nuclear Factor Kappa B regulatory element (NFkB-RE), Octamer Motif (OCT), Retinoic Acid regulatory element (RARE) and Yin Yang 1 regulatory element (YY1-RE). The sequences of these elements are provided in Table 1, SEQ ID NOs 1-28.

In one embodiment, the two or more transcription factor regulatory elements are independently selected from the group consisting of NFκB-RE, E-box, AP1, CRE, GC-Box, E41F, C/EBPα-RE, OCT and RARE.

In one embodiment, the two or more transcription factor regulatory elements are independently selected from the group consisting of NFκB-RE, E-box, CRE, GC-Box, E41F and C/EBPα-RE.

In one embodiment, the two or more transcription factor regulatory elements are independently selected from the group consisting of NFκB-RE (SEQ ID NO:25), E-box (SEQ ID NO:11), GC-box (SEQ ID NO:13) and C/EBPα-RE (SEQ ID NO:4).

Advantageously, these 4 transcription factor regulatory elements are significantly associated with high expressing promoter constructs. Hence, promoter constructs which comprise these particular transcription factor regulatory elements are capable of double the transcriptional activity of the CMV promoter.

In a one example, the two or more transcription factor regulatory elements are independently selected from the group consisting of NFκB-RE, E-box and GC-box.

In a one example, the two or more transcription factor regulatory elements are independently selected from the group consisting of NFκB-RE, E-box and C/EBPα-RE.

In a further embodiment, the two or more transcription factor regulatory elements are independently selected from the group consisting of NFκB-RE and E-box.

In a further embodiment, the two or more transcription factor regulatory elements are independently selected from the group consisting of NFκB-RE and CRE.

In one embodiment the transcription factor regulatory element is not YY1.

Advantageously, these combinations of 2 or 3 transcription factor regulatory elements are predominant in the high expressing promoter constructs of the present invention and are therefore likely to exert a particularly strong influence on expression levels.

Thus, in one embodiment the synthetic promoters employed provide increased levels of expression in comparison to known promoters, in particular wild-type CMV promoters, for example a 5, 10, 15, 20, 25, 30, 35, 40, 45, 50, 55, 60, 65, 70, 75, 80, 85, 90, 95, 100% or more increase in expression levels.

In another embodiment, the synthetic promoters, for example in a library or collection are employed to provide a specific activity across three orders of magnitude. This enables optimized transcriptional activity for specific purposes, for example, selection marker expression, antibody heavy chain:light chain ratio control, and polypeptide-specific control of transcription for difficult to express proteins.

Thus, in one embodiment the synthetic promoters employed provide reduced levels of expression in comparison to known promoters, in particular wild-type CMV promoters. However, these promoters will generally be chosen because they have an advantageous property over and above a known promoter, such as allowing more protein to be produced with appropriate folding or similar.

In one embodiment, the transcription factor regulatory elements have a nucleotide sequence selected from the group consisting of SEQ ID NOs: 1 to 28, for example the transcription factory regulatory elements have a nucleotide sequence selected from the group consisting of SEQ ID NOs: 1, 4, 6, 8, 11, 13, 25, 26 and 27, such as a nucleotide sequence selected from the group consisting of SEQ ID NOs: 4, 11, 13 and 25, in particular a nucleotide sequence selected from the group consisting of SEQ ID NOs: 11 and 25. In one embodiment, the synthetic promoter comprises 2 to 50 transcription factor regulatory elements, for example 2, 3, 4, 5, 6, 7, 8, 9, 10, 12, 13, 14, 15, 16, 17, 18, 19 or 20 transcription factor regulatory elements.

Advantageously, the use of two or more different synthetic promoter sequences to control the expression of two or more recombinant genes (for example heavy chain and light chain genes) may help to minimize gene copy loss. In standard current expression systems two identical CMV promoters are used to control expression of both genes, which can lead to gene copy loss via homologous recombination. In yet another embodiment, each promoter comprises 4, or 5, or 6, or 7, or 8 or 9 or 10 or 11 or 12 or more transcription factor regulatory elements.

The synthetic promoters of the present disclosure may incorporate more types of transcription factor regulatory element, thereby further increasing the range of transcription activities that can be obtained.

In one embodiment, the core promoter is derived from a promoter selected from the group consisting of CMV, SV40, UbC, EF1A, PGK and CAGG, such as CMV.

In one embodiment the promoter or core promoter is not EAAT2, in particular is not SEQ ID NO: 1, 2 and/or 3 disclosed in WO03/070965. In one embodiment the promoter or core promoter is not derived from EAAT2.

In one embodiment the promoter or promoter core is not a COX-2 gene promoter. In one embodiment the promoter or core promoter is not derived from a COX-2 gene promoter.

Advantageously, the inventors have found that the transcriptional activity of the CMV core promoter can be reliably modified by including blocks of transcription factor regulatory elements upstream of the CMV core promoter. Hence, the CMV core promoter (SEQ ID NO:170) is particularly suited for use in a synthetic promoter construct of the present invention.

In another embodiment, the core promoter is derived from an inducible promoter, for example selected from the group consisting of TRE, PTRE3G and c-Fos.

Advantageously, the use of an inducible promoter as a core promoter offers a further level of control of the transcriptional activity of the promoter constructs of the present invention by allowing control of the timing of expression of a recombinant protein.

In one embodiment, a synthetic promoter of the present invention comprises a nucleotide sequence selected from the group consisting of SEQ ID NOs: 30 to 169.

Advantageously, these synthetic promoters are composed of up to 7 transcription factor regulatory elements independently selected from NFκB-RE, E-box, AP1-RE, CRE, GC-box, E4F1-RE and C/EBPα-RE, which the inventors have discovered are particularly active in CHO cells. When incorporated into synthetic promoters of the present invention, in any orientation either 5′ to 3′ or 3′ to 5′ these TFREs result in a wide range of different transcriptional activities.

In one embodiment the synthetic promoter according to the present disclosure comprises part or all of the proximal promoter from CMV, for example 10, 20, 30, 40, 50% of the proximal promoter.

In one embodiment the synthetic promoter according to the present disclosure comprises part or all of the distal promoter from CMV, for example 10, 20, 30, 40, 50% of the distal promoter.

In one embodiment the synthetic promoter according to the present disclosure comprises part or all of the proximal promoter from CMV and part or all of the distal promoter, for example independently 10, 20, 30, 40, 50% of the proximal promoter and proximal promoter.

In one example the synthetic promoter comprises a TFRE construct consisting of two or more, such as 3, 4, 5, 6, 7, 8, 9, 10 or more copies of NFκB-RE and two or more such as 3, 4, 5, 6, 7, 8, 9, 10 or more copies of E-box and optionally one or more copies of GC-Box and/or C/EBPα-RE.

In a one embodiment, a synthetic promoter of the present invention comprises a nucleotide sequence selected from the group consisting of SEQ ID NOs: 30 to 169.

In one example, a synthetic promoter of the present invention comprises a nucleotide sequence selected from the group consisting of SEQ ID NOs: 126 to 169.

In one example, a synthetic promoter of the present invention comprises a nucleotide sequence selected from the group consisting of SEQ ID NOs: 126 to 150. In one example, a synthetic promoter of the present invention comprises a nucleotide sequence selected from the group consisting of SEQ ID NOs: 30, 31, 32, 126, 128 and 144.

Advantageously, the transcription factor regulatory element constructs with SEQ ID NOs: 126 to 169 were constructed using solely the 4 transcription factor regulatory elements NFκB-RE, E-box, GC-box and C/EBPα-RE. Accordingly, the expression levels obtained using these TFREs tend to be significantly higher compared to the constructs with SEQ ID NOs: 30 to 125.

In another aspect, there is provided a library of mammalian cell-specific synthetic promoter constructs, comprising a plurality of synthetic promoters as defined herein.

In another aspect, there is provided a library of transcription factor regulatory element constructs with, for example SEQ ID NOs 30-169, such as 126 to 169.

Advantageously, a library of transcription factor regulatory element constructs of the present invention and a library of synthetic promoter constructs containing them designed for expressing recombinant proteins in CHO cells provides a repository of available promoter constructs with a varying range of levels of expression. The libraries provide a convenient resource from which a user can readily select a promoter construct with the desired levels of expression or test for a desired level of expression for any given protein.

In one embodiment there is provided a plasmid or vector comprising a synthetic promoters sequence according to the present disclosure.

In one embodiment the plasmid or vector further comprises a polynucleotide sequence, such as a gene encoding a recombinant protein or transcribable polynucleotide sequence. The latter includes RNAi, shRNA and other useful polynucleotide sequences.

In one embodiment the plasmid or vector may comprise a pair of restriction sites, for example sandwiching the polynucleotide sequence encoding a recombinant protein or transcribable polynucleotide. The restriction sites facilitate exchange of the genetic material in the plasmid or vector.

As discussed above the present disclosure also extends to a mammalian cell, such as a CHO cell comprising a synthetic promoter described herein.

In one embodiment the cell is transiently transfected with: a synthetic promoter of the associated polynucleotide (eg gene) of interest, a plasmid or vector according to the present disclosure.

In one embodiment the cell is stably transfected with a synthetic promoter according to the present disclose, for example wherein the synthetic promoter is associated with polynucleotide (eg gene) of interest.

Also provided is a library of cells, for example CHO cells, stably transfected with a range of synthetic promoters according to the present disclosure. A range of synthetic promoters as employed herein is intended to refer to where generally each cell contains a different synthetic promoter or a different combination of synthetic promoters stably integrated into its genome.

In one aspect, there is provided a mammalian host cell, comprising a synthetic promoter or an expression vector as defined above. In one embodiment, the mammalian host cell is selected from the group consisting of a Chinese Hamster Ovary (CHO) cell, a mouse myeloma cell and a hybridoma cell. Examples of suitable cell types include lymphocytic cell lines, e.g., NS0 myeloma cells and SP2 cells and COS cells. Suitable types of CHO cells for use in the present invention may include CHO and CHO-K1 cells including dhfr-CHO cells, such as CHO-DG44 cells and CHO-DXB11 cells and which may be used with a DHFR selectable marker or CHOK1-SV cells which may be used with a glutamine synthetase selectable marker. In one embodiment the CHO is independently selected from CHO-S, CHO-K1 and CHO-DG44.

In one embodiment the host cell, such as a CHO cell, has increased levels of expression of Ero1 and/or XBP1 in comparison to a wild-type cell, for example as disclosed in WO2010/092335 incorporated herein by reference.

In one embodiment the host cell, such as a CHO cell, is transfected with an NHEJ protein (non-homologous end joining protein) or functional fragment thereof, as disclosed in PCT/EP2013/070317 incorporated herein by reference.

In one example there is provided a CHO cell comprising a synthetic promoter suitable for eliciting recombinant protein expression therein said synthetic promoter comprising a promoter core and upstream thereof at least one TFRE construct wherein each TFRE construct consists of two or more transcription factor regulatory elements independently selected from the group consisting of NFκB-RE, E-box, AP1, CRE, GC-Box, E41F, C/EBPα-RE, OCT and RARE.

In one aspect, there is provided a CHO cell comprising two or more synthetic promoters as described above, for example directly combined to create novel chimeras. Advantageously, this provides an easy way to modify the combination of transcription factor regulatory elements which are present by inserting additional promoters into an expression vector to be transfected into the cell.

In one aspect, there is provided a CHO cell comprising two or more synthetic promoters as described above, where each is used to direct the synthesis of a different recombinant protein, for example the heavy and light chains of an antibody or antigen binding fragment thereof. It will be appreciated that where these are incorporated into a vector these different synthetic promoters may be on the same or different vectors.

In one embodiment there is also provided a library of plasmids, vectors or cells each comprising a synthetic promoter according to the present disclosure. As discussed above the present disclosure also extends to a library of host cells comprising said synthetic promoter(s) or plasmids/vectors of the present the disclosure.

In one example the present invention provides a method of identifying a synthetic promoter suitable for promoting recombinant protein expression in a given mammalian recombinant host cell comprising the steps of:
a) obtaining a library of two or more synthetic promoters each comprising a promoter core and upstream thereof a transcription factor regulatory element construct comprising or consisting of a sequence selected from any one of SEQ ID NOs: 30 to 169.
b) testing the library of synthetic promoter constructs obtained in step a) to determine the level of expression of recombinant protein driven by each construct in the chosen mammalian recombinant host cell
c) selecting a synthetic promoter construct tested in step (b) if it promotes recombinant protein expression at the desired level.

In a further aspect there is provided a method of optimising the promoter and cell combination for the expression of a particular protein. Thus there is provided a method comprising the step of i) analysing at least one parameter, such as levels of expression, of at least two host cells (such as at least two CHO cells) each comprising a synthetic promoter according to the presently disclosure, for the same protein under comparable conditions.

In one embodiment the method further comprises the step of selecting the cell and synthetic promoter combination, which is most suited to the expression of the given protein.

Different as employed in this context is intended to refer to wherein the combination of elements in the cell are not the same (non-identical) to the cell it is being compared to, for example if the genome of the cells being compared is essentially identical then the synthetic promoters in the cells will be different (or another entity in the cells will be different). If the synthetic promoters in said at least two cells is the same then some element, for example an element or gene in the cells genome will be different (non-identical).

Thus the method provides for comparison of at least two cells typically when the analysis is performed under similar conditions, for example the analysis is performed at essentially the same time or in the same experiment.

It will be appreciated that further libraries may be constructed using the TFRE elements described herein above and employed to generate synthetic promoters and promoter libraries and screened as described herein above. In one example the present invention provides a method of constructing a TFRE construct library comprising the step of randomly ligating together the TFRE elements NFκB-RE, E-box, GC-Box and C/EBPα-RE. In one example the library is constructed where, for example these elements are randomly ligated at a ratio of 5:3:1:1.

In one embodiment the present invention provides a method of constructing a TFRE construct library comprising the step of randomly ligating together the TFRE elements NFκB-RE and E-box and/or CRE. In one example the library is constructed where, for embodiment these elements are randomly ligated at a ratio of 5:3.

In one embodiment the library of synthetic promoters is specifically designed (rational design) with different permutations of various combinations of TFREs, for example as disclosed herein.

As discussed above promoters with a range of activities can be prepared employing the disclosure herein. The provision of discrete promoters with activities, covering over three orders of magnitude, allows CHO cell engineers to precisely engineer a suitable cell factory, where systems level control of cell function may require the constitutive expression of several genes to be stoichiometrically balanced.

In one embodiment the synthetic promoters of the present disclosure, plasmids comprising same and cells comprising the promoter, plasmid or vector, such as mammalian cells, in particular CHO cells are suitable for driving transcription of a polynucleotide, for example DNA or RNA, for example RNAi, shRNA and the like.

In one aspect there is provided a method of generating a synthetic promoter suitable for promoting recombinant protein expression in a given mammalian recombinant host cell comprising the steps of:
a) identifying motifs of transcription factor regulatory elements,
b) testing each transcription factor regulatory elements identified in step a) in combination with a promoter core for activity in a chosen mammalian recombinant host cell line,
c) selecting two or more transcription factor regulatory elements which are more active in the chosen mammalian recombinant host cell line than the promoter core alone,
d) preparing one or more synthetic promoter constructs comprising two or more of those transcription factor regulatory elements independently selected from those selected in step c),
e) testing the construct or constructs prepared in step d) for activity in the chosen mammalian recombinant host cell, and
f) identifying the synthetic promoter construct or constructs that exhibit improved protein expression compared to a wild type promoter, such as the wild type promoter from which the promoter core is derived.
   In one embodiment, the method further comprises the steps of:
g) selecting two or more of those transcription factor regulatory elements which are associated with the constructs identified in step f), and
h) preparing one or more synthetic promoter constructs comprising a TFRE construct comprising or consisting of those elements independently selected in step g).

Advantageously, the additional steps allow further refinement of the synthetic promoter constructs produced by the above method, thereby resulting in promoter constructs for a target host cell line that exhibit enhanced expression, for example enhanced levels of expression.

In one embodiment, the synthetic promoter constructs prepared in step h) comprise TFRE constructs consisting of transcription factor regulatory elements at a stoichiometry which reflects their relative abundance in the constructs identified in step f).

Advantageously, by incorporating the various transcription factor regulatory elements at a stoichiometry which mirrors their relative abundance in high expressing synthetic promoters, this further distils and helps to isolate the transcription factor regulatory elements that are more likely to enhance the basal transcriptional activity levels of the core promoter.

In one embodiment the method further comprises the steps of identifying the transcription factor regulatory element or elements that are most frequently associated with promoter constructs which exhibit reduced activity compared to the wild type promoter and not selecting these in step (g).

In the methods described above ‘activity’ is typically the level of protein expression driven by any given promoter, for example the level of recombinant protein or reporter gene expression.

In a related aspect, there is provided a method of synthesising a promoter of the present invention, comprising the steps of:
a. analysing the expression levels of a reporter gene in a mammalian cell line, wherein the reporter gene expression is driven by a plurality of synthetic promoters, wherein each promoter comprises one or more transcription factor regulatory element constructs, located upstream of a core promoter, and wherein each TFRE construct consists of a single type of transcription factor regulatory element;
b. comparing the reporter gene expression levels driven by the synthetic promoters with the reporter gene expression level driven by a control promoter which has a known level of activity;
c. identifying the synthetic promoters with a reporter gene expression level that is higher than the expression level of the control promoter construct, thereby identifying the transcription factor regulatory elements that are active in the mammalian cell line; and
d. selecting the transcription factor regulatory elements identified in step c and incorporating two or more of those elements into a transcription factor regulatory element construct and incorporating said construct into a synthetic promoter, wherein each transcription factor regulatory construct consists only of the selected transcription factor regulatory elements.

Advantageously, the above methods allow the synthesis of promoters of the present invention where there is no a priori knowledge of which particular transcription factor regulatory elements would be active in and thus suitable for a target host cell line.

In a related embodiment, the above method further comprises the steps of:
e. analysing the expression levels of a reporter gene in a mammalian cell line, wherein the reporter gene expression is driven by a plurality of synthetic promoters from step d;
f. identifying the promoters that have the highest and lowest levels of expression;
g. determining the relative frequency of each of the transcription factor regulatory elements present in the promoter constructs from step f, thereby identifying the transcription factor regulatory elements that are associated with higher expression levels; and
h. selecting the transcription factor regulatory elements identified in step g and incorporating those elements into a synthetic promoter comprising one or more transcription factor regulatory constructs, wherein each construct consists only of the selected transcription factor regulatory elements.

Clearly the above disclosed method can be modified to replace the level of protein expression with another desirable parameter, for example activity of the expressed protein in a functional assay or the appropriateness/level of the protein folding.

A plurality of synthetic promoters as employed herein refers to analogous cell populations each containing a different synthetic promoter or different combination of synthetic promoters, so that the expression levels from the different populations can be compared.

In one embodiment the recombinant protein is an antibody or binding fragment thereof, for example as discussed in more details below.

Advantageously, the additional steps allow further refinement of the synthetic promoter constructs produced by the above method, thereby resulting in promoter constructs for a target host cell line that exhibit enhanced levels of expression.

In one embodiment, the transcription factor regulatory elements are incorporated into the promoter construct in step h at a stoichiometry which is derived from their relative representation in the synthetic promoter constructs from step d.

Advantageously, by incorporating the various transcription factor regulatory elements at a stoichiometry which mirrors their relative abundance in high expressing synthetic promoters, this further distils and helps to isolate the transcription factor regulatory elements that are more likely to enhance the basal transcriptional activity levels of the core promoter.

### DESCRIPTION OF FIGURES

FIG. 1 shows a graph depicting the results of a reporter gene assay to identify transcription factor regulatory elements which are active in CHO cells. 28 transcription factor regulatory elements (TFREs) (see Table 1) were derived from informatics analysis of transcription factor binding sites in common viral promoters known to be active in mammalian cells.
The 28 TFREs were assessed for their transcriptional activities in CHO-S cells (sequence list shown in Table 2) using SEAP/GFP reporter constructs. A schematic diagram of the general structure of the reporter construct is also shown in FIG. 1. Seven copies of each TFRE (as described in Table 1) were cloned in series upstream of a minimal CMV core promoter in reporter vectors encoding either GFP or SEAP reporters.
CHO-S cells (2×10⁵) in 24-well plates were transfected with 1 μg of SEAP (black bars) or GFP (white bars) TFRE reporter-vector. SEAP activity in cell culture supernatant and intracellular GFP were measured 24 h post-transfection. Data are expressed as a fold-change with respect to the activity of a vector containing only a minimal CMV core promoter (Core). A random 8 bp sequence with no known homology to TFRE sequences (8mer) was also used as a −ve control. Bars represent the mean+SD of three independent experiments each performed in triplicate, using three clonally derived plasmids for each TFRE-reporter construct.

FIG. 2 shows a graph which indicates some of the results of a reporter gene assay comparing the expression levels of a selection of Generation 1 synthetic promoters of the present invention (sequences shown in Table 3).
First generation synthetic promoters were constructed by random ligation of NFκB, CRE, E-box, GC-box, E4F1 and C/EBPα TFREs in equal proportion.

Synthetic promoters were inserted upstream of a minimal CMV core promoter in SEAP reporter plasmids and transfected into CHO-S cells. FIG. 2 includes a schematic diagram of the general structure of the Generation 1 synthetic promoters.
SEAP expression was quantified 24 h post-transfection. Data are expressed as a percentage of the production exhibited by promoter 1/01 (see Table 3 for sequence). SEAP production from the control CMV-SEAP reporter is shown as the black bar. Each bar represents the mean of two transfections, for each promoter less than 10% variation in SEAP production was observed.

FIG. 3A/B shows a series of graphs, each of which shows the results of an analysis of the abundance of a TFRE relative to the expression levels of the Generation 1 promoter constructs in which the TFRE is present.
The number of each TFRE in each synthetic promoter is plotted against the relative activity of that promoter (A-F). In each case the linear regression line is shown, where the slope of the line indicates the extent to which each TFRE occurs in promoters of varying activity.
Over-mean: higher than average expression level (i.e. high expressing constructs).
Under-mean: lower than average expression level (i.e. low expressing constructs).

FIG. 4 shows a graph indicating some of the results of a reporter gene assay comparing the expression levels of a selection of Generation 2 synthetic promoter constructs of the present invention (sequences shown in Table 4).
Second generation synthetic promoters were constructed by random ligation of NFκB, E-box, GC-box, C/EBPα TFREs in the ratio 5:3:1:1. Synthetic promoters were inserted upstream of a minimal CMV core promoter in SEAP reporter plasmids and transfected into CHO-S cells. SEAP expression was quantified 24 h post-transfection. Data are expressed as a percentage of the production exhibited by CMV control promoter (black bar). SEAP production from the most active promoter from the first generation library (1/01; FIG. 2) reporter is shown as a checked bar. Otherwise, each bar represents the mean of two transfections, for each promoter less than 10% variation in SEAP production was observed.
Promoter construct 1/01 (hatched bar): top Generation 1 promoter which produced the highest levels of expression.

FIG. 5 shows a series of graphs, each of which shows the results of an analysis of the abundance of a TFRE relative to the expression levels of the Generation 2 promoter constructs in which the TFRE is present.
The number of each TFRE in each synthetic promoter is plotted against the relative activity of that promoter (A-D). In each case the linear regression line is shown, where the slope of the line indicates the extent to which each TFRE occurs in promoters of varying activity.
Over-mean: higher than average expression level (i.e. high expressing constructs).
Under-mean: lower than average expression level (i.e. low expressing constructs).

FIG. 6 shows the results of reporter gene assays indicating the relative activity of seven synthetic promoters with differing relative activity was determined in CHO-S cells, CHO-K1 cells and CHO-DG44 cells. Cells (2×10⁵) were transfected with 250 ng SEAP-reporter vector, and SEAP production was quantified 24 h post-transfection. Data are expressed as a percentage of the activity of the CMV promoter in each cell line. Values represent the mean+S.D of three independent experiments performed in triplicate.
Interestingly the relative performance of each promoter was not the same in all cells lines tested.

FIG. 7 shows the results of reporter gene assays involving longer term transient expression (i.e. performed over 7 days) in fed-batch culture of CHO-S cells using the same synthetic promoter constructs depicted in FIG. 6.
CHO-S cells (6×10⁶) were transfected with 7.5 μg of SEAP reporter-vectors, where SEAP expression was under the control of synthetic promoters with varying activity or the control CMV promoter. SEAP production and viable cell concentration were measured over the course of a 7-day fed-batch process in tube-spin bioreactors. The mean IVCD (integral of viable cell density) at Day 7 (white bars) and SEAP titer (black bars) are shown. SEAP data are expressed as a percentage of the control CMV promoter activity. Two independent transfections were performed in duplicate.

FIG. 8 shows various sequences of synthetic promoters according to the present disclosure

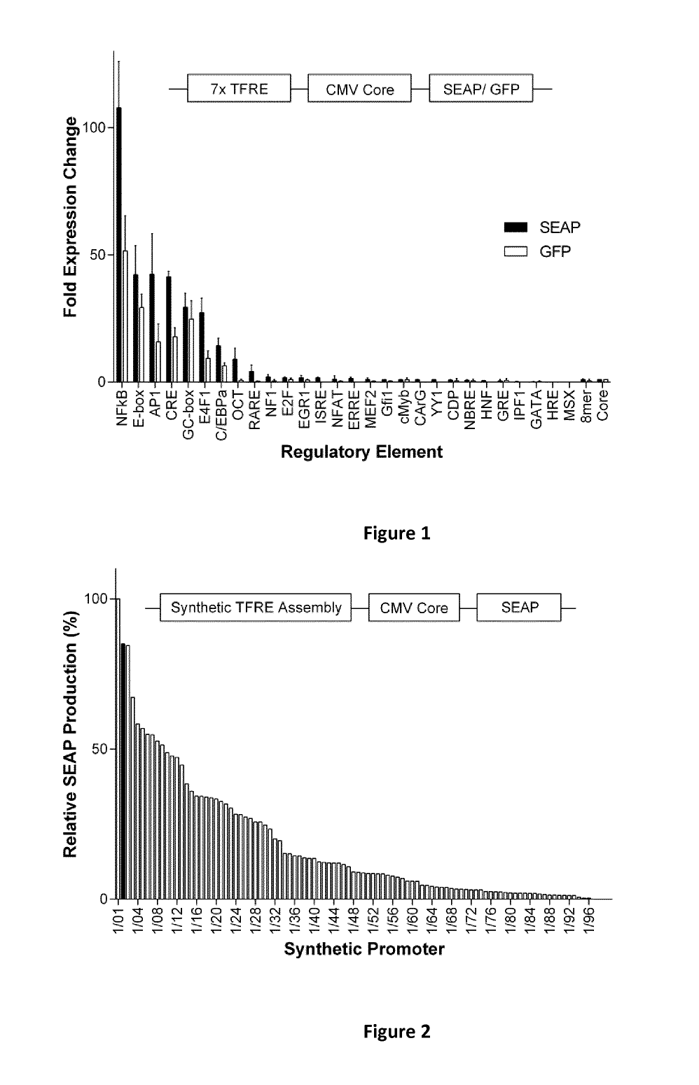

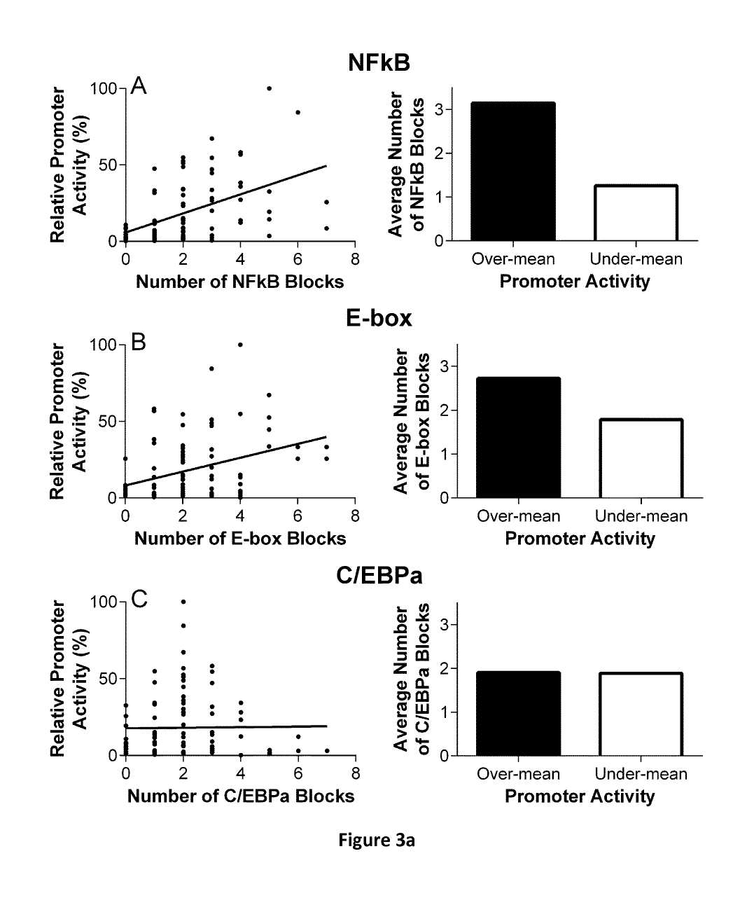

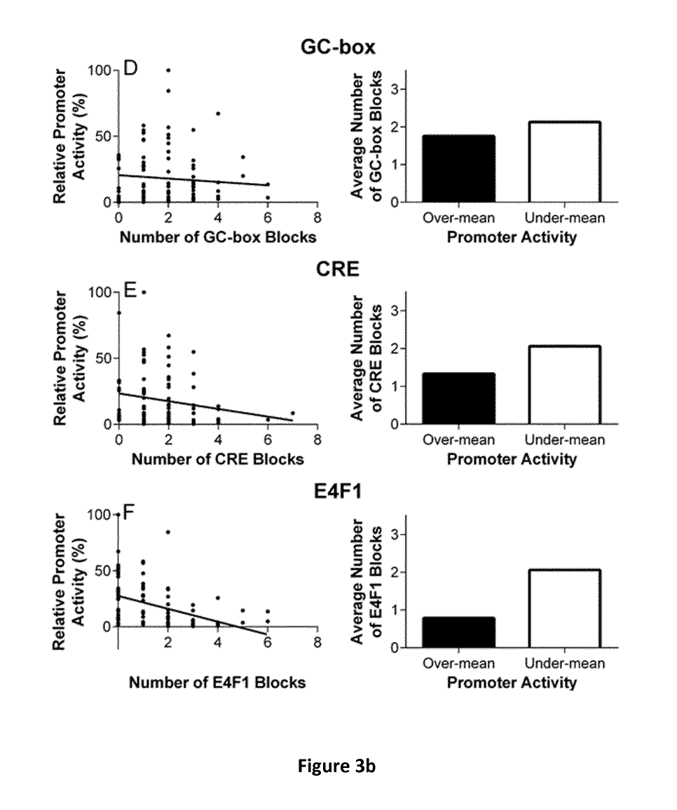

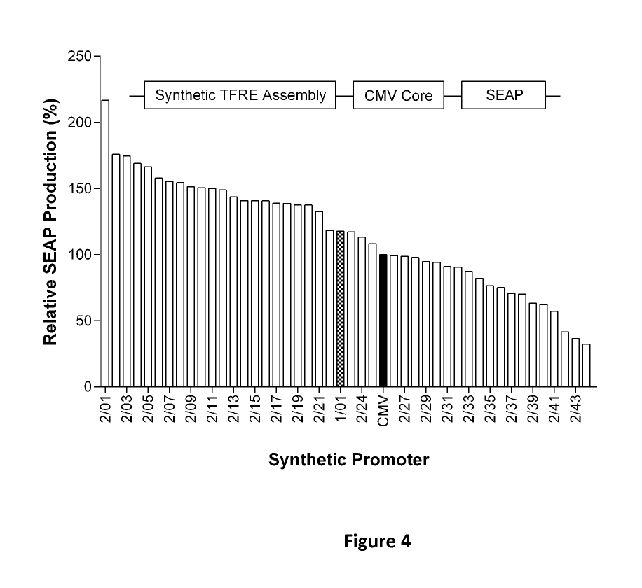

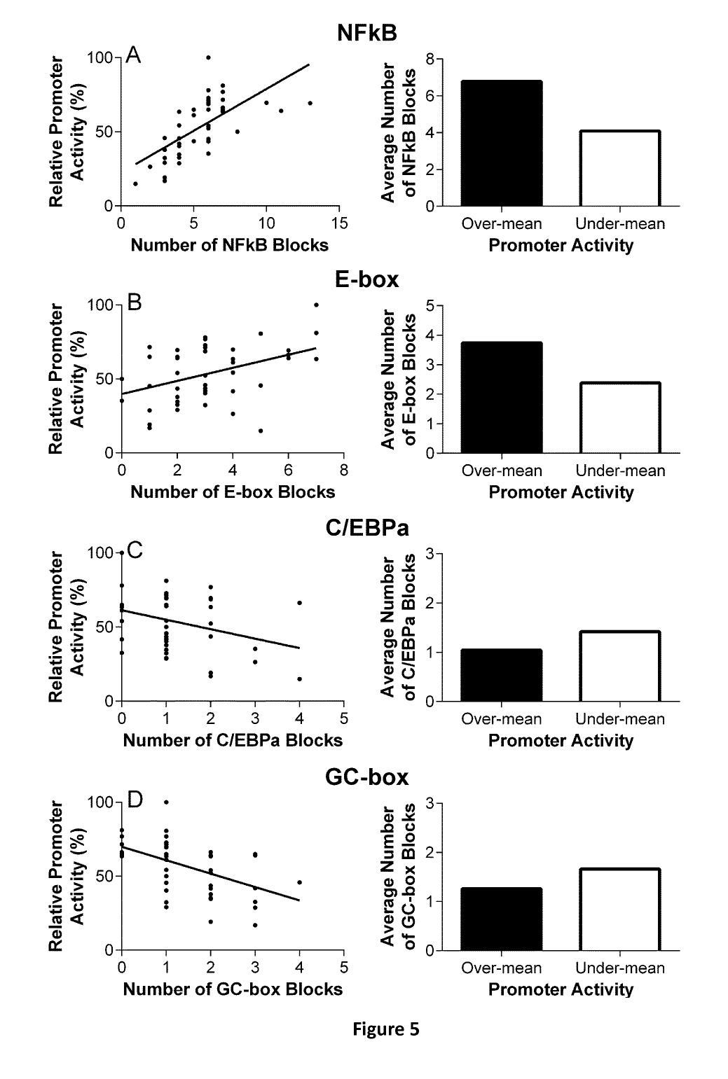

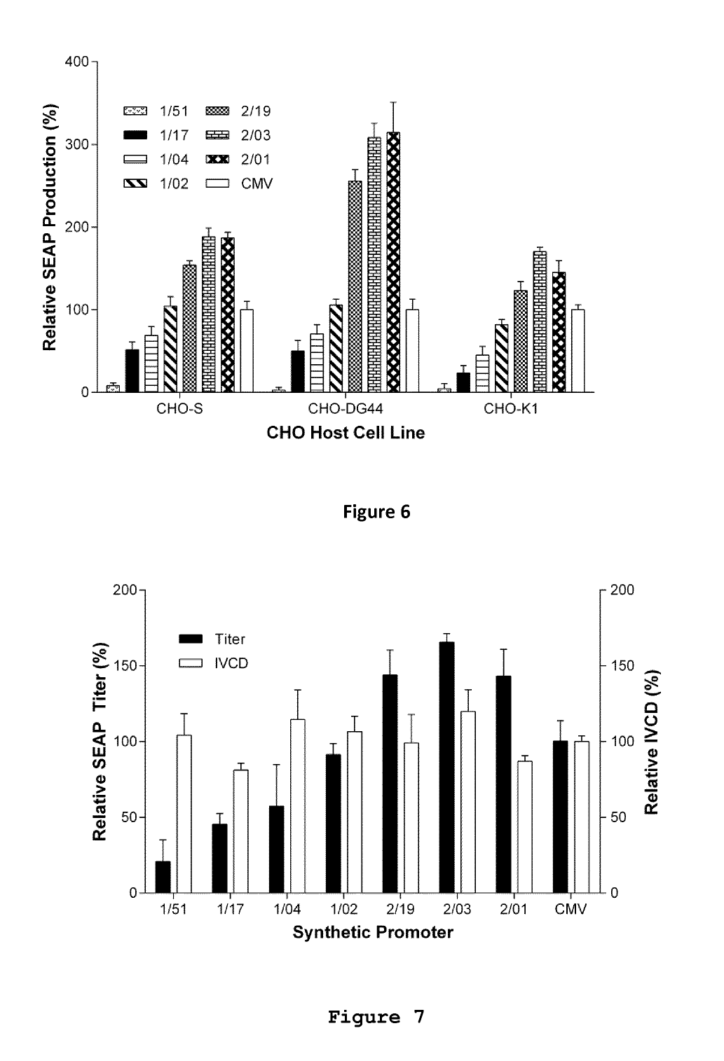

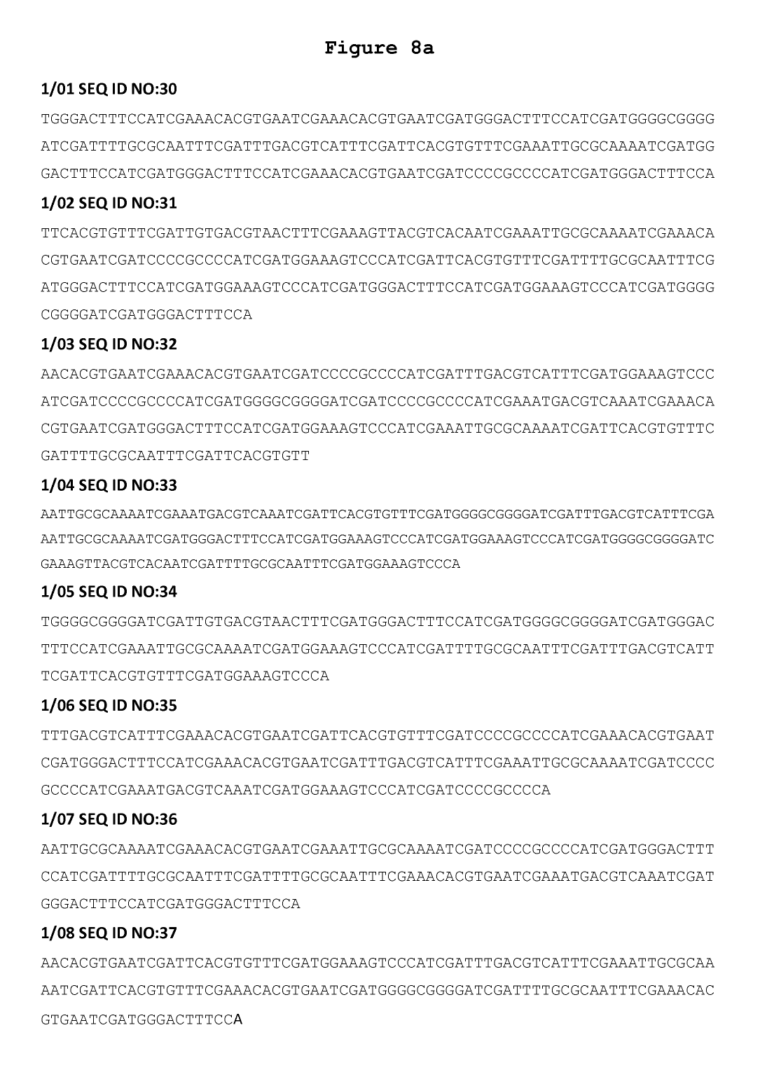

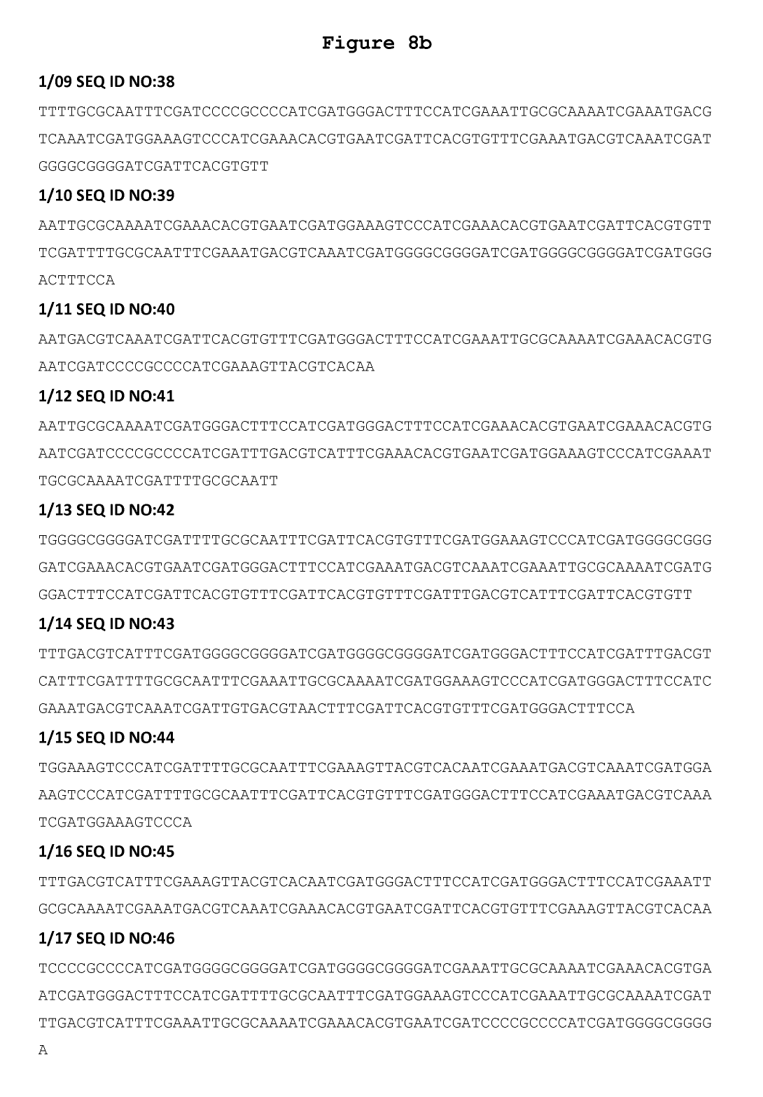

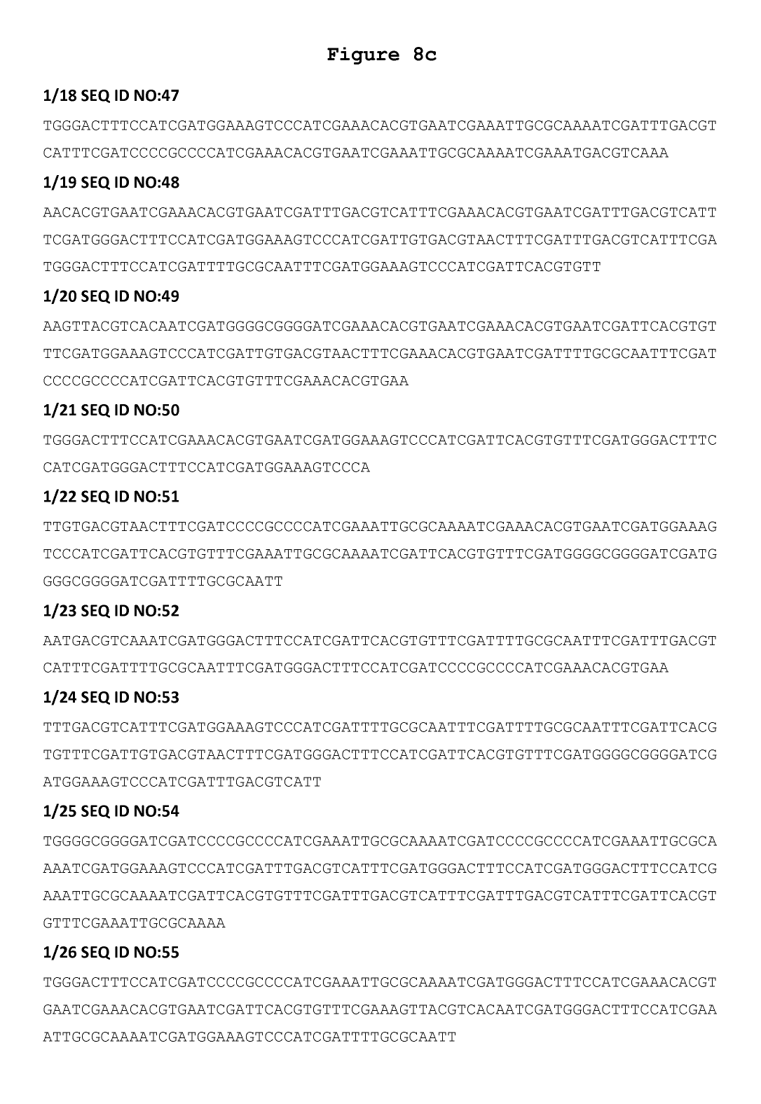

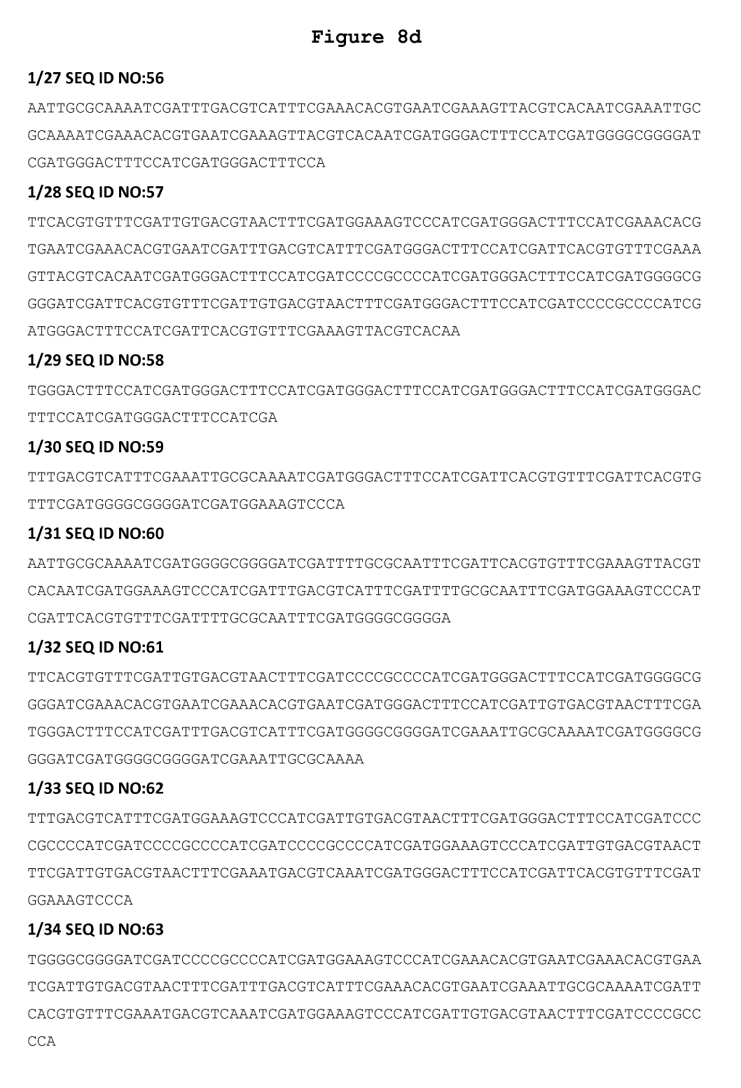

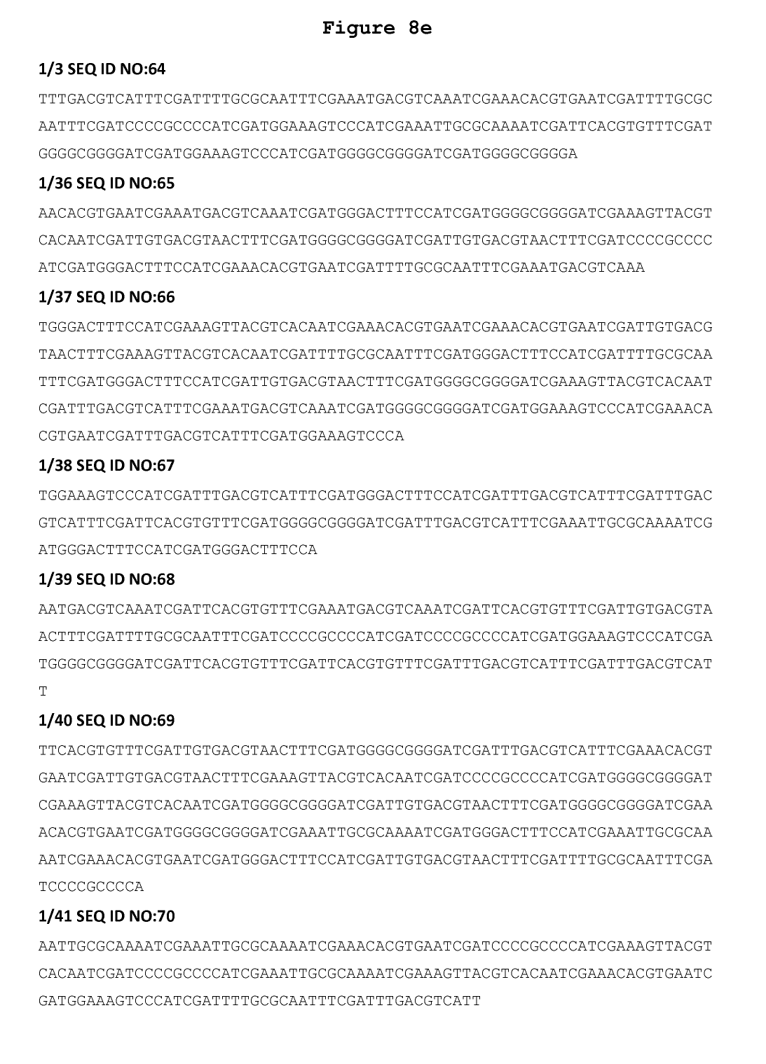

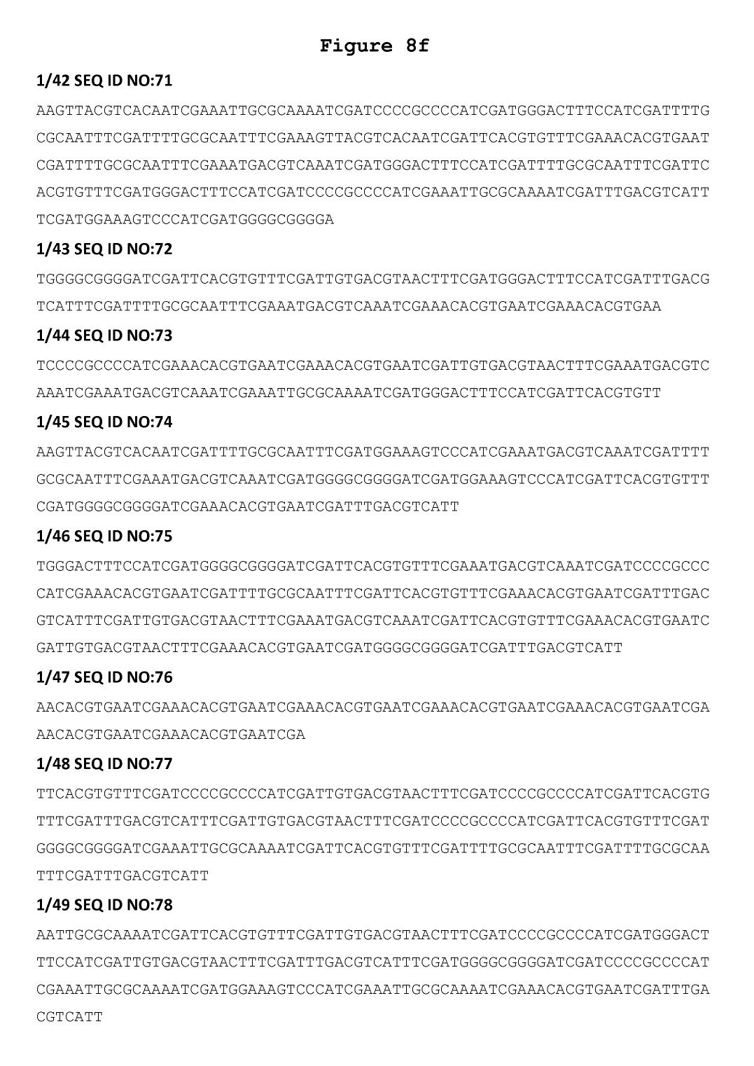

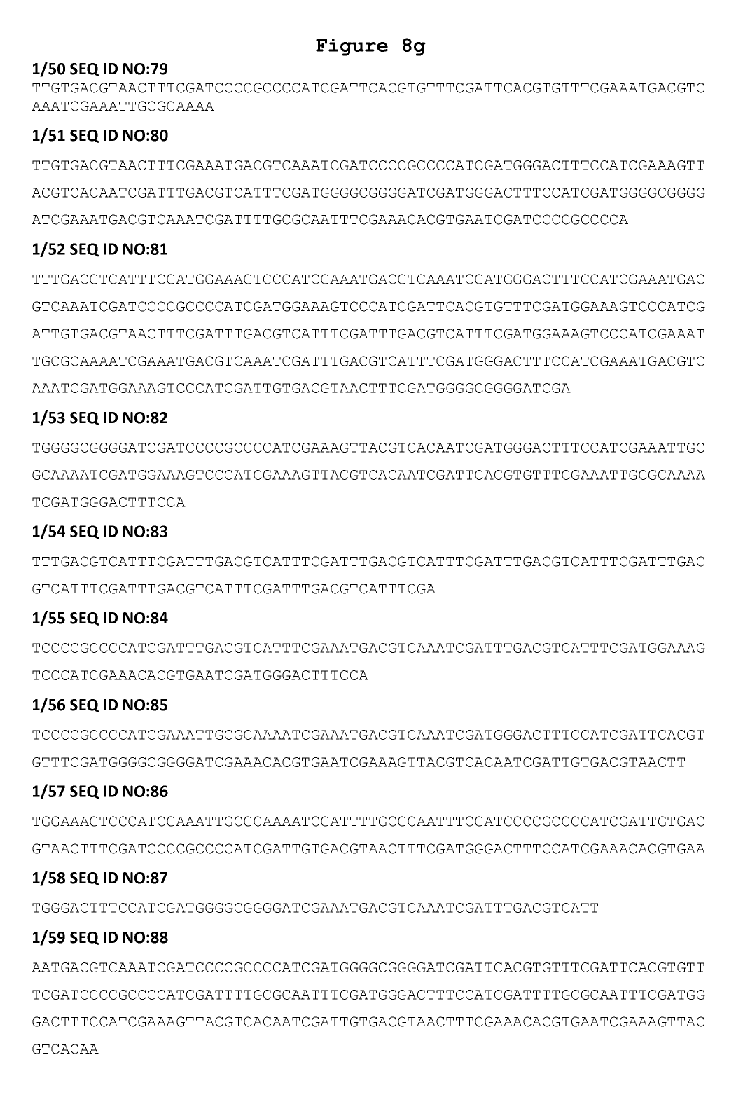

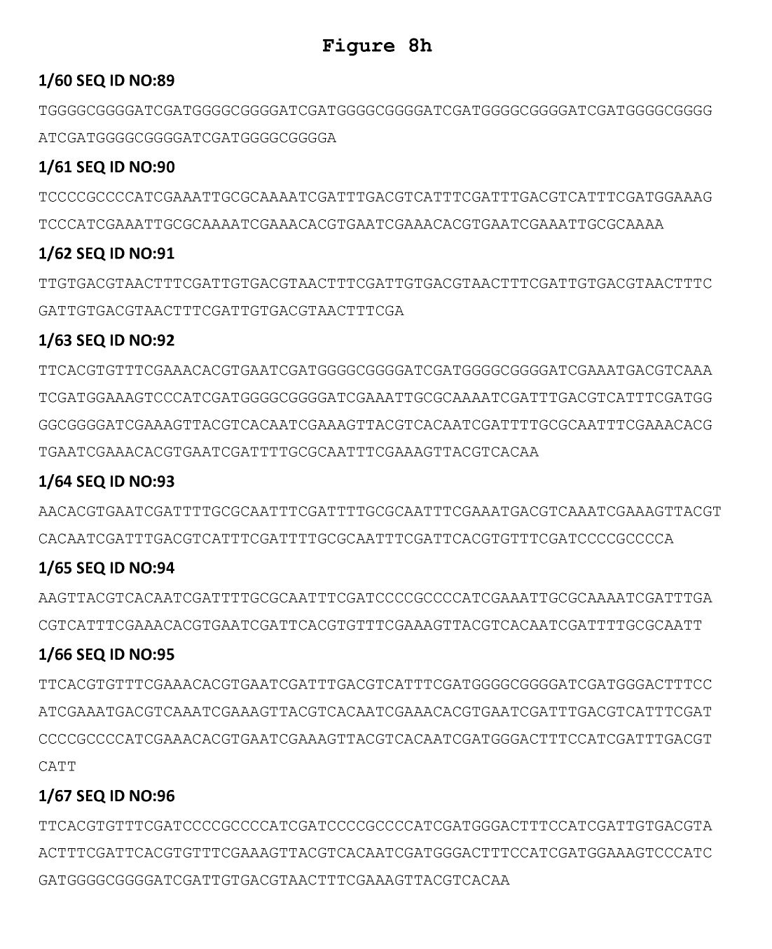

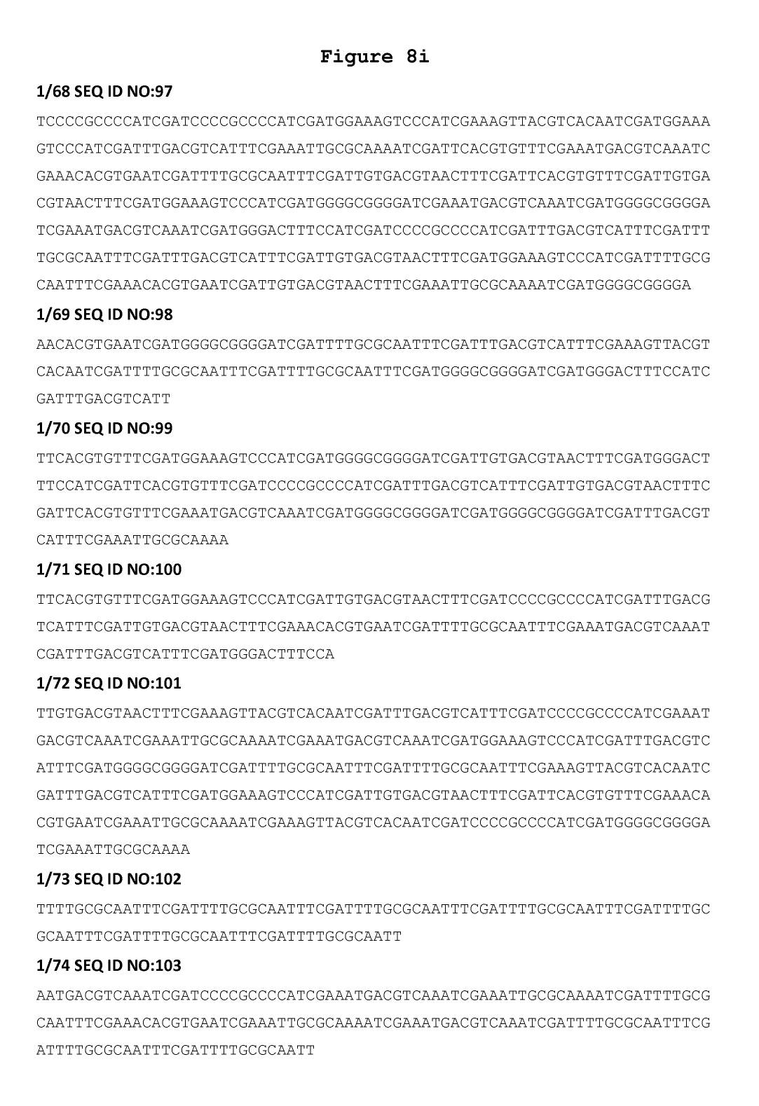

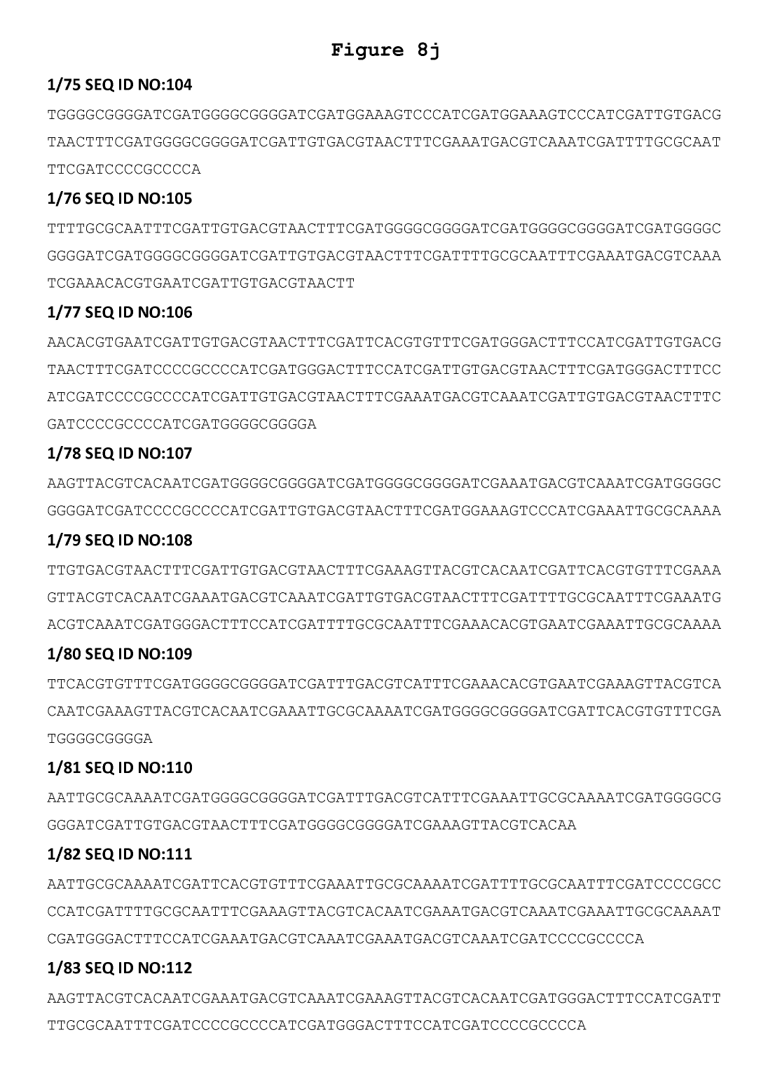

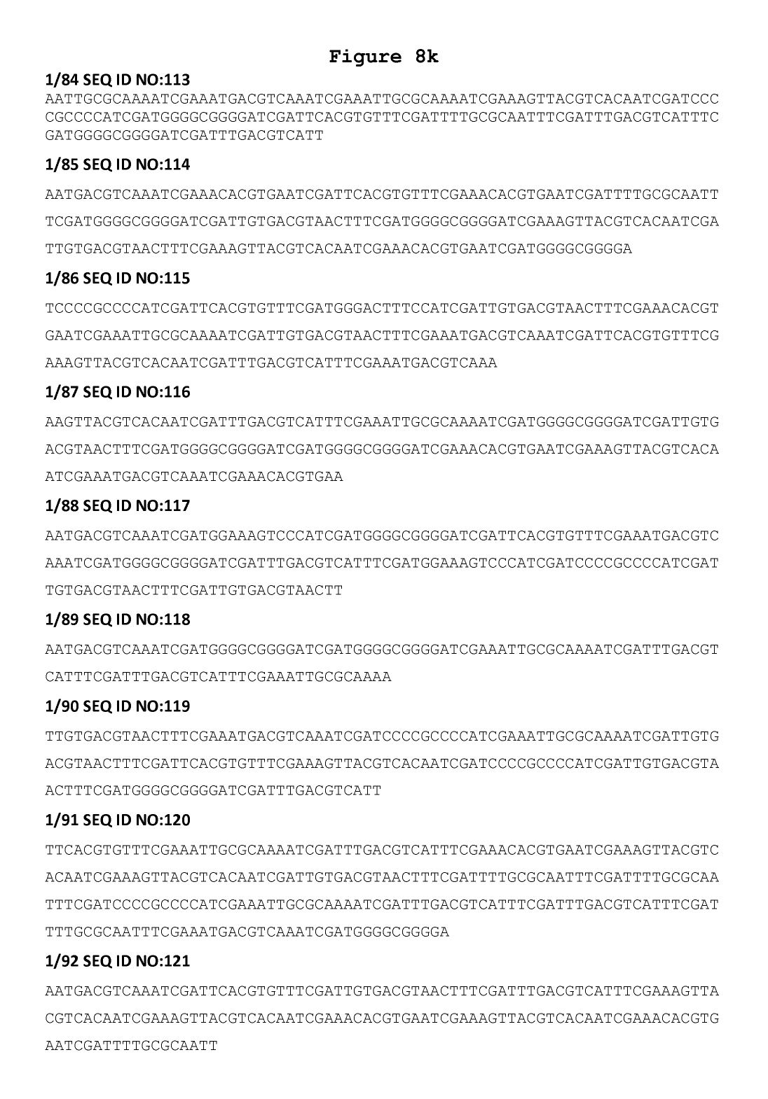

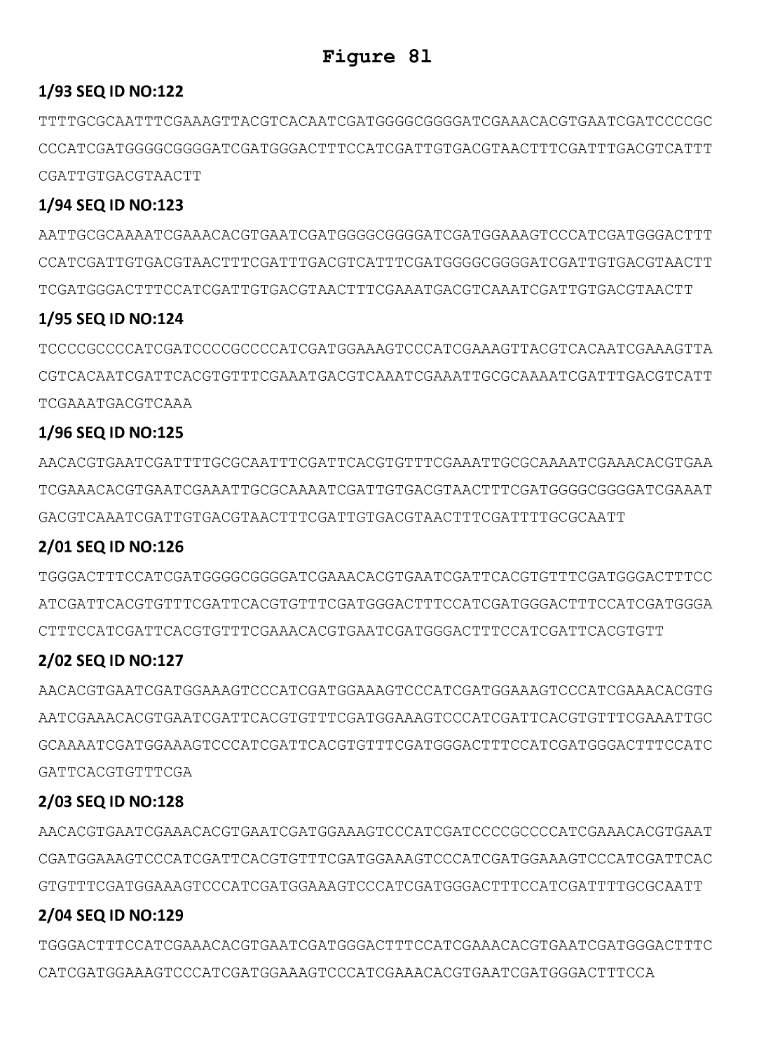

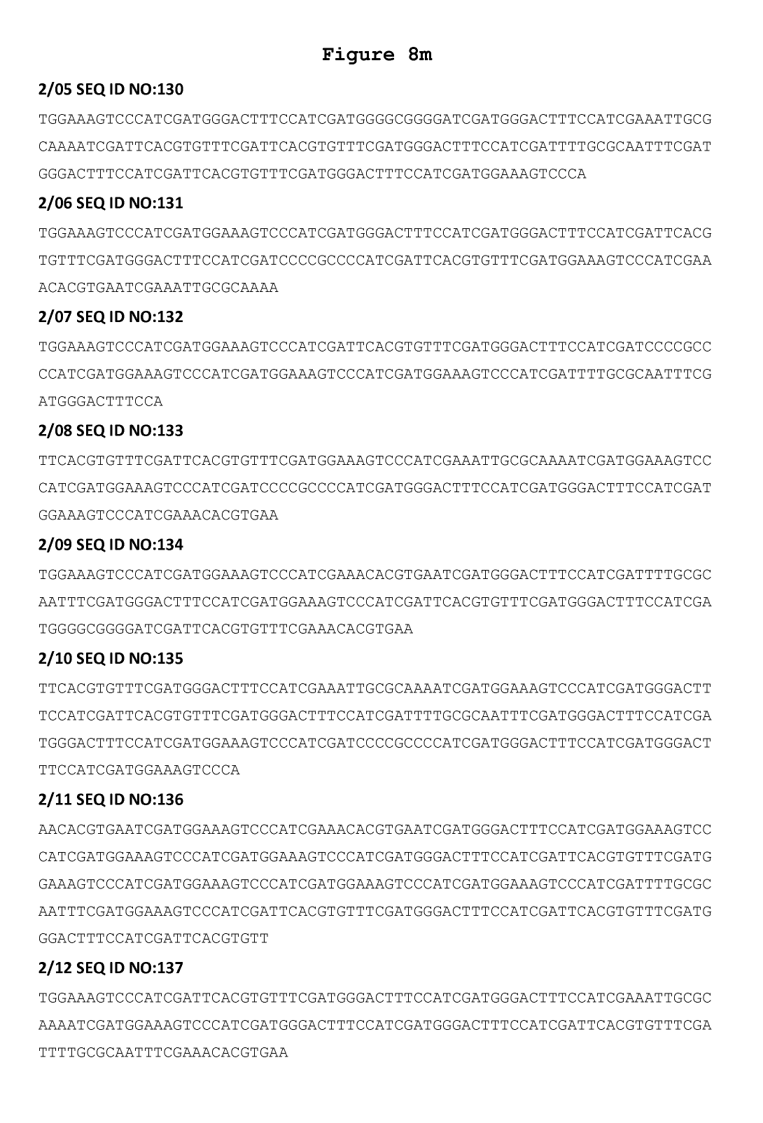

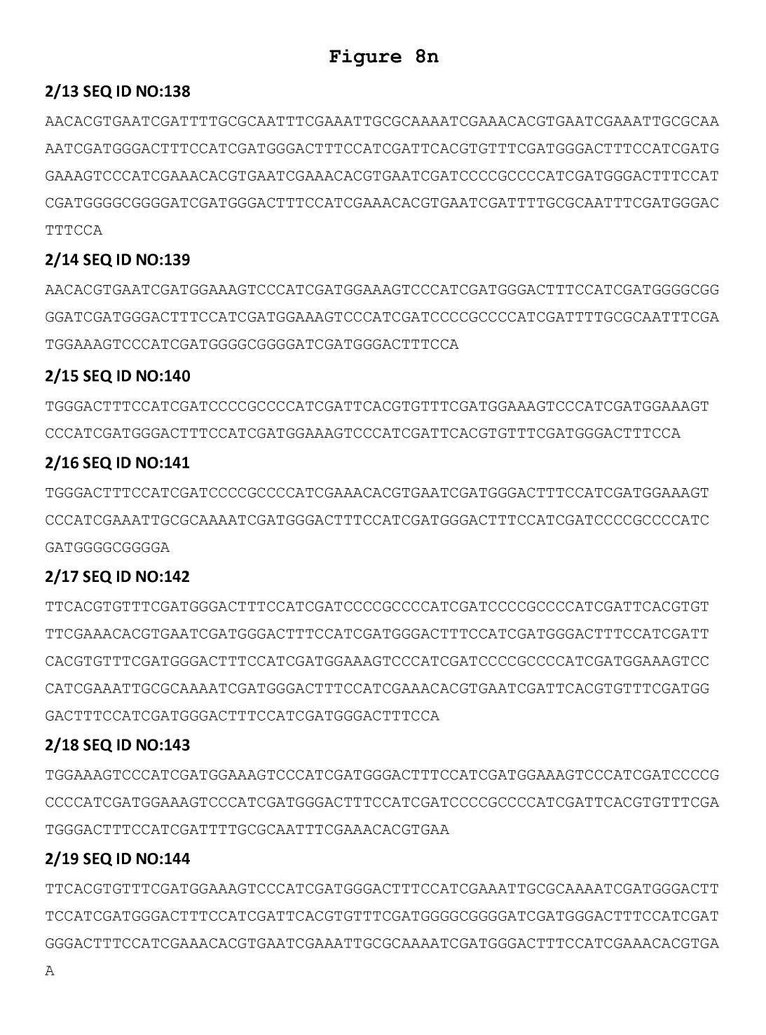

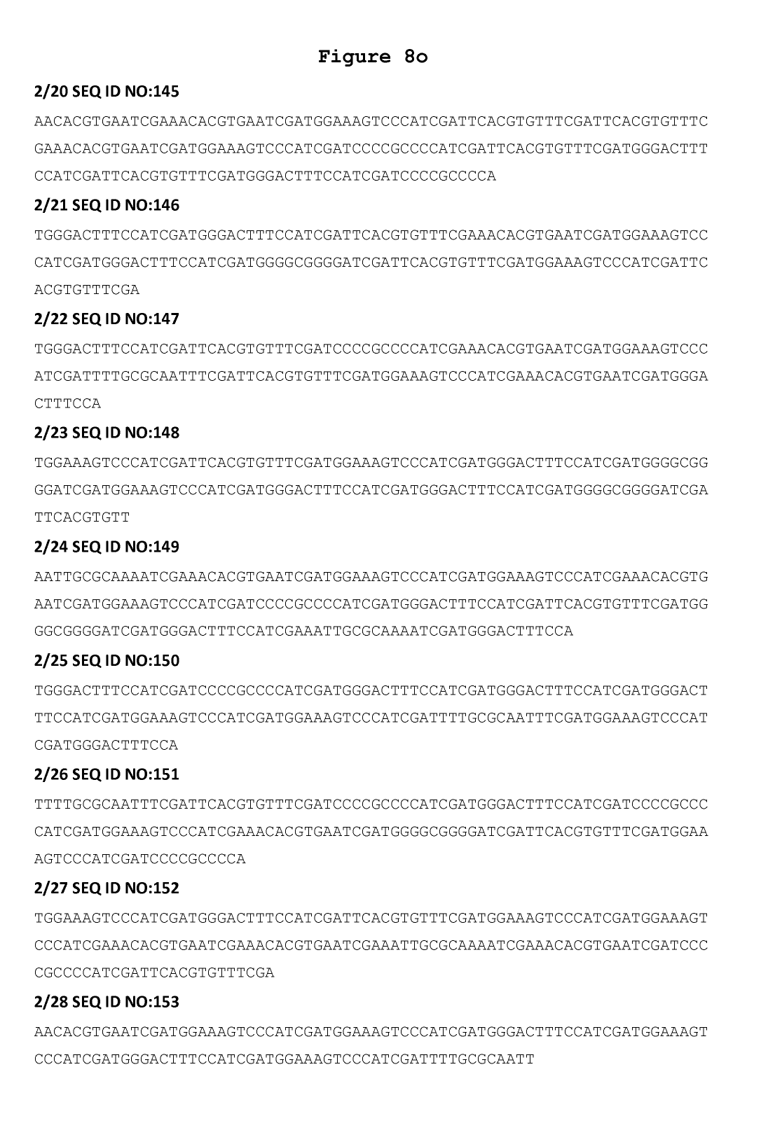

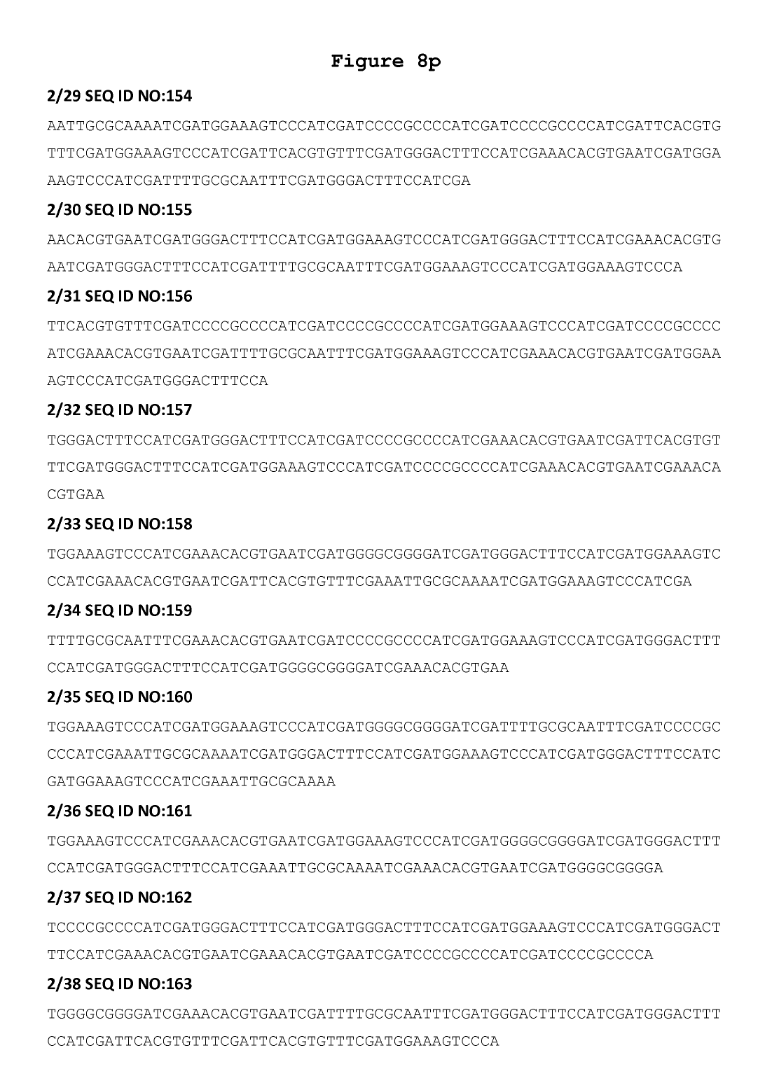

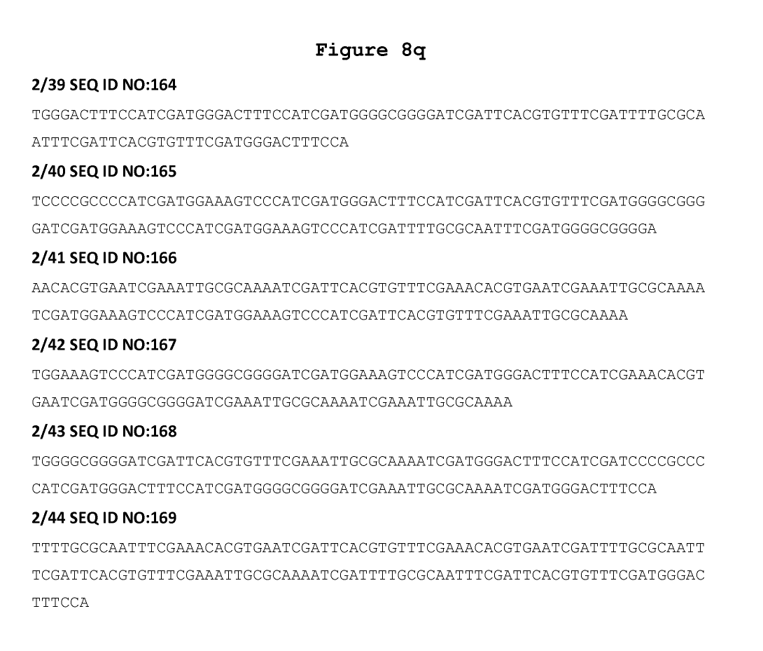

### DETAILED DESCRIPTION OF INVENTION

“In tandem” as employed herein refers to sequences “in line” one after the other.

As used herein, the term “transcription factor” refers to any cellular factor, including proteins that bind to a cis-acting region and regulate either positively or negatively the expression of the gene, for example, a transcription factor may bind upstream of the coding sequence of a gene to either enhance or repress transcription of the gene by assisting or blocking RNA polymerase binding. Transcription factors or repressors or co-activators or co-repressors, and the like are encompassed within this definition.

As used herein, the term “transcription factor regulatory element”, or “TFRE”, or “regulatory element” refers to a nucleotide sequence that is recognized and bound by a transcription factor.

A TFRE comprises a nucleic acid sequence suitably, a double stranded DNA sequence. A TFRE may comprise a cis-acting region and may also comprise additional nucleic acids. The core six to eight nucleotides of promoter and enhancer elements may be sufficient for the binding of their corresponding transcription factors.

Thus, a TFRE may consist of 6 to 8 nucleic acid bases. A TFRE of the invention may be 6 or more, 8 or more, 10 or more, 15 or more, 20 or more, 25 or more, or 30 or more bases in length. A TFRE of the invention may be 100 or less, 75 or less, 50 or less, 30 or less, 25 or less, 20 or less or 15 or less bases in length.

The person of skill in the art will understand, however, that influence from other transcription factors, e.g. general transcription factors or transcription factors binding promiscuously to polynucleotides, is not always excluded. Thus, a synthetic promoter construct wherein at least 50%, at least 60%, at least 70%, at least 80%, at least 90%, at least 95%, or at least 99% of transcription activity or repression activity is mediated by the transcription factors for which transcription factor regulatory elements have been incorporated into said promoter is to be considered to be specifically controlled by the transcription factors.

A particularly suitable TFRE is one that is active in the cell or tissue of interest. Such a TFRE may be identified as being associated with a gene that is expressed in the cell or tissue of interest, for example, a TFRE may be associated with a gene that is differentially expressed in that cell or tissue, when compared with another cell or tissue. Differential expression of a gene may be seen by comparing the expression of the gene in two different cells or tissues, or in the same cells or tissues under different conditions.

Expression in one cell or tissue type may be compared with that in a different, but related, tissue type, for example, where the cell or tissue of interest is a disease cell or tissue or has been artificially manipulated as described herein, the expression of genes in that cell or tissue may be compared with the expression of the same genes in an equivalent normal or untreated cell or tissue. This may allow the identification of genes that are differentially regulated between the two cell or tissue types.

A TFRE that is associated with such a gene is generally located close to the coding sequence of the gene within the genome of the cell, for example, such a TFRE may be located in the region immediately upstream or downstream of that coding sequence. Such a TFRE may be located close to a promoter or other regulatory sequence that regulates expression of the gene. The location of a TFRE may be determined by the skilled person using a variety of known methods, such as those described in the present specification.

Some suitable examples of transcription factor regulatory elements of the present invention are shown in Table 1 below.

TABLE 1
Transcription Factor Regulatory Elements: Ten viral promoters thought
to exhibit activity in CHO cells were surveyed for the presence
of discrete transcription factor regulatory elements (transcription
factor binding sites) using Transcription Element Search System
(TESS) and Transcription Affinity Prediction (TRAP) algorithms
using stringent sequences of single TFREs that occur in search
parameters to minimize false positives. DNA of their relative
ability to activate more than one viral promoter are listed.
Measurement cells is shown in FIG. 1.
transcription of recombinant reporter genes in CHO-S

| | | SEQ |
|---|---|---|
| Transcription Factor Regulatory Element | Sequence | ID NO: |
| Activator protein 1 (AP1) | TGACTCA | 1 |
| CC(A/T)₆GG element (CArG) | CCAAATTTGG | 2 |
| CCAAT displacement protein (CDP) | GGCCAATCT | 3 |
| CCAAT-enhancer binding protein alpha | TTGCGCAA | 4 |
| (C/EBPα) | | |
| Cellular myeloblastosis (cMyb) | TAACGG | 5 |
| cAMP RE (CRE) | TGACGTCA | 6 |
| Elongation factor 2 (E2F) | TTTCGCGC | 7 |
| E4F1 | GTGACGTAAC | 8 |
| Early growth response protein 1 (EGR1) | CGCCCCCGC | 9 |
| Estrogen-related receptor alpha RE (ERRE) | AGGTCATTTTGACCT | 10 |
| Enhancer box (E-box) | CACGTG | 11 |
| GATA-1 (GATA) | AGATAG | 12 |
| GC-box | GGGGCGGGG | 13 |
| Glucocorticoid RE (GRE) | AGAACATTTTGTTCT | 14 |
| Growth factor independence 1 (Gfi1) | AAAATCAAC | 15 |
| Helios RE (HRE) | AATAGGGACTT | 16 |
| Hepatocyte nuclear factor 1 (HF) | GGGCCAAAGGTCT | 17 |
| Insulin promoter factor 1 (IPF1) | CCCATTAGGGAC | 18 |
| Interferon-stimulated RE (ISRE) | GAAAAGTGAAACC | 19 |
| Myocyte enhancer factor 2 (MEF2) | CTAAAAATAG | 20 |
| Msx homeobox (MSX) | CGGTAAATG | 21 |
| Nerve growth factor-induced gene-B RE | AAAGGTCA | 22 |
| (NBRE) | | |
| Nuclear factor 1 (NF1) | TTGGCTATATGCCAA | 23 |
| Nuclear factor of activated T cells (NFAT) | AGGAAATC | 24 |
| Nuclear factor kappa B (NFκB) | GGGACTTTCC | 25 |
| Octamer motif (OCT) | ATTAGCAT | 26 |
| Retinoic acid RE (RARE) | AGGTCATCAAGAGGTCA | 27 |
| Yin yang 1 (YY1) | CGCCATTTT | 28 |
| Random 8mer (8mer) [-ve control] | TTTCTTTC | 29 |

It will be understood that the transcription regulatory elements of the invention are not limited to specific sequences referred to in the specification but also encompass their structural and functional analogs/homologues. Such analogs may contain truncations, deletions, insertions, as well as substitutions of one or more nucleotides introduced either by directed or by random mutagenesis. Truncations may be introduced to delete one or more binding sites for known transcriptional repressors.

Additionally, such sequences may be derived from sequences naturally found in nature that exhibit a high degree of identity to the sequences in the invention. A nucleic acid of about 20 nucleotides or more will be considered to have high degree of identity to a transcription factor regulatory element of the invention if it hybridizes to the relevant transcription factor under stringent conditions. Alternatively, a nucleic acid will be considered to have a high degree of identity to a transcription factor regulatory element of the invention if it comprises a contiguous sequence of about 20 or more nucleotides, which has percent identity of at least 70%, 75%, 80%, 85%, 90%, 95%, or more as determined by standard alignment algorithms such as, for example, Basic Local Alignment Tool (BLAST) described in Altshul et al., J. Mol. Biol. 1990, 215: 403-410, the algorithm of Needleman et al., J. Mol. Biol. 1970, 48: 444-453, or the algorithm of Meyers et al., Comput. Appl. Biosci. 1988, 4: 11-17.

The transcription factor regulatory elements of the present invention may be chosen from transcription factor regulatory elements already known to be active in a target host cell or may be putative regulatory elements determined by in silico analysis of sequences upstream of core promoters, by using methods known to those of skill in the art.

In one embodiment there is provided use of a combination of transcription factor regulatory element combination selected from a group described herein, for example use to improve transcription, translation or expression in host, in particular by incorporation into a synthetic promoter.

In one embodiment there is provided use of a synthetic promoter, for example as described herein for driving expression in a mammalian host cell, such as a CHO cell.

These in silico analyses typically operate by comparing non-coding regulatory sequences between the genomes of various organisms to enable the identification of conserved regions that are significantly enriched in promoters of candidate genes or from clusters identified by microarray analysis and can potentially function as transcription factor regulatory elements. Examples of these software suites include TRAFAC, CORG, CONSITE, CONFAC, VAMP and CisMols Analyser.

As used herein, the term “promoter” or “promoter construct” refers to a DNA segment that contains components for an efficient transcription of a gene and includes one or more transcription factor regulatory elements; a core promoter region; and optionally, sequences from 5′-untranslated region or introns.

As used herein, the term “synthetic promoter construct” or “synthetic promoter” refers to an artificial or engineered or assembled promoter sequence, for example comprising two or more transcription factor regulatory elements, such as containing one or more transcription factor regulatory element constructs.

As used herein, the term “transcription factor regulatory element construct” or “TFRE construct” refers to an assembled double stranded DNA molecule that comprises more than one transcription regulatory element sequence. The construct may be created by a number of different means that would be known to the skilled addressee; including the ligation of various double stranded transcription regulatory elements together in a random or directed fashion. The construct may comprise other nucleic acid sequences as well, e.g. spacers that do not mediate binding of a transcription factor but allow for a correct spatial arrangement of binding sites. The spacer region, for example may be a common overhang which allows different TFREs to be easily ligated to each other.

The TFREs are usually in tandem with one another, but may be separated by an overhang sequence.

The construct may comprise at least two transcription factor regulatory elements that are the same.

The construct may comprise at least two different transcription factor regulatory elements for at least two different transcription factors.

The construct may also comprise a plurality, for example, two, three, four, five, six, seven, eight, nine, or ten or more transcription factor regulatory elements. A number of these may be the same or they may all be different.

When assembled within a construct, the transcription factor regulatory elements bind multiple transcription factors expressed in the target host cells and efficiently drive expression of a recombinant protein or reporter protein. However, the same combined transcription factor regulatory elements may be inactive in non-target host cells due to the lack of particular transcription factors required for binding to the sequence elements. Thus, the combinatorial nature of gene transcription is most effectively utilized, by knowing the exact profile of transcription factors and co-regulators that are active in the target host cells.

As used herein, the term “core promoter” or “promoter core” refers to a short DNA segment which is the minimal portion of the promoter required to initiate transcription. Core promoter sequence can be derived from various different sources, including prokaryotic and eukaryotic genes. Examples of this are dopamine beta-hydroxylase gene minimum promoter and cytomegalovirus (CMV) immediate early gene promoter. In one example, the core promoter is derived from a promoter selected from the group consisting of CMV, SV40, UbC, EF1A, PGK and CAGG, such as CMV.

A core promoter may be “inducible”, wherein transcription is initiated in response to an inducing agent or an increased level of transcription of an operatively linked expressible polynucleotide as compared to the level of transcription, if any, in the absence of an inducing agent.

Alternatively, a promoter may be “constitutive”, wherein the transcription activity is not affected by the presence of an inducing agent.

The term “inducing agent” is used to refer to a chemical, biological or physical agent that effects transcription from an inducible promoter. An inducing agent can be, for example, a stress condition to which a cell is exposed, for example, a heat or cold shock, a toxic agent such as a heavy metal ion, or a lack of a nutrient, hormone, growth factor, or the like; or can be exposure to a molecule that affects the growth or differentiation state of a cell such as a hormone or a growth factor.

A core promoter may also be regulated in a “tissue-specific” or “tissue-preferred” manner, such that it is only active in transcribing the operable linked coding region in a specific tissue type.

As used herein, the term ‘wild type promoter’ refers to a promoter sequence as it occurs in nature.

Unless stated otherwise, the term “CMV promoter” refers to the commonly acknowledged full length hCMV-IE1 promoter (i.e. the core and the proximal elements) GenBank accession number: M60321.1, bases 595-1193). The term “CMV core” or “hCMV-IE1 core” as used herein refers to the minimal hCMV-IE1 core promoter provided herein as SEQ ID NO:170.

The term “operatively linked” as used herein refers to elements or structures in a nucleic acid sequence that are linked by operative function (i.e. able to influence the function or respond to the function of the other element) and not physical location. Hence, it is not necessary for elements or structures in a nucleic acid sequence to be in a tandem or adjacent order to be operatively linked.

The term “vector” as used herein refers to any vehicle that delivers a nucleic acid into a cell or organism. An example of a vector is a “plasmid,” which is a circular double stranded DNA loop into which additional DNA segments may be ligated and can replicate independently of chromosomal DNA. Plasmids occur or are derived from mainly bacteria and sometimes from other microorganisms. However, mitochondrial and chloroplast DNA, yeast killer and other cases are commonly excluded.

Another type of vector is a viral vector, wherein additional DNA segments may be ligated into the viral sequence. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell, where they are subsequently replicated along with the host genome. In the present specification, the terms “plasmid” and “vector” may be used interchangeably as a plasmid is the most commonly used form of vector.

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

The vectors of the present invention may comprise a selectable marker, which is a protein whose expression allows one to identify cells that have been transformed or transfected with a vector containing the marker gene.

A wide range of selection markers are known in the art, for example, the selectable marker may be a gene for neomycin phosphotransferase (npt II), which expresses an enzyme conferring resistance to the antibiotic kanamycin, and genes for the related antibiotics neomycin, paromomycin, gentamicin, and G418, or the gene for hygromycin phosphotransferase (hpt), which expresses an enzyme conferring resistance to hygromycin.

The term “expression vector” as used herein, refers to a vector encoding a recombinant protein that is to be expressed in a target host cell. A plurality of different expression vectors as described herein may be provided. These may form a library.

As used herein, a “host cell” is a cell comprising one or more synthetic promoters, vectors or expression vectors of the present invention. The cell may be a mammalian cell. The host cell may be a cultured cell or a body cell. Suitable mammalian host cells include CHO, myeloma or hybridoma cells.

Transfection of vectors into the target host cells of the present invention may be achieved using any suitable method. A variety of transfection methods are known in the art and the skilled person will be able to select a suitable method depending on the type of vector and type of host cell desired.

The term “transient expression” as used herein refers to the capacity of a host cell to direct the transcription and translation of recombinant genetic sequences. This transcription and translation occurs soon after the genetic sequences are introduced into the host cell. Such expression can occur even when a plasmid which carries a reporter gene sequence operably linked to a functional promoter is introduced into a host cell incapable of replicating or propagating the plasmid.

As used herein, a “recombinant protein” refers to a protein that is constructed or produced using recombinant DNA technology. The protein of interest may be an exogenous sequence identical to the endogenous protein or a mutated version thereof, for example with attenuated biological activity, or fragment thereof, expressed from an exogenous vector. Alternatively, the protein of interest may be a heterologous protein, not normally expressed by the host cell.

The recombinant protein may be any suitable protein including therapeutic, prophylactic or diagnostic protein.

The recombinant protein expressed under the control of a synthetic promoter according to the invention may, for example be an immunogenic protein, a fusion protein comprising two heterologous proteins or an antibody. Antibodies for use as the recombinant protein include monoclonal, multi-valent, multi-specific, humanized, fully human or chimeric antibodies. The antibody may be a complete antibody molecule having full length heavy and light chains or a fragment thereof, e.g. VH, VL, VHH, Fab, modified Fab, Fab′, F(ab′)₂, Fv or scFv fragment.

After expression antibody fragments may be further processed, for example by conjugation to another entity or for example the antibody fragments may be PEGylated to generate a product with the required properties, for example similar to the whole antibodies, if required.

Examples of antigens of interest bound by the antibodies or fragments thereof may include, but are not limited to, any medically relevant protein such as those proteins upregulated during disease or infection, for example receptors and/or their corresponding ligands. Particular examples of cell surface proteins include adhesion molecules, for example integrins such as β1 integrins e.g. VLA-4, E-selectin, P selectin or L-selectin, CD2, CD3, CD4, CD5, CD7, CD8, CD11a, CD11b, CD18, CD19, CD20, CD23, CD25, CD33, CD38, CD40, CD45, CDW52, CD69, CD134 (OX40), ICOS, BCMP7, CD137, CD27L, CDCP1, DPCR1, DPCR1, dudulin2, FLJ20584, FLJ40787, HEK2, KIAA0634, KIAA0659, KIAAl246, KIAA1455, LTBP2, LTK, MAL2, MRP2, nectin-like2, NKCC1, PTK7, RAIG1, TCAM1, SC6, BCMP101, BCMP84, BCMP11, DTD, carcinoembryonic antigen (CEA), human milk fat globulin (HMFG1 and 2), MHC Class I and MHC Class II antigens, and VEGF, and where appropriate, receptors thereof.

Soluble antigens include interleukins such as IL-1, IL-2, IL-3, IL-4, IL-5, IL-6, IL-8, IL-12, IL-16, IL-23, viral antigens for example respiratory syncytial virus or cytomegalovirus antigens, immunoglobulins, such as IgE, interferons such as interferon α, interferon β or interferon γ, tumor necrosis factor-β, colony stimulating factors such as G-CSF or GM-CSF, and platelet derived growth factors such as PDGF-α, and PDGF-13 and where appropriate receptors thereof. Other antigens include bacterial cell surface antigens, bacterial toxins, viruses such as influenza, EBV, HepA, B and C, bioterrorism agents, radionuclides and heavy metals, and snake and spider venoms and toxins.

The term “reporter gene” as used herein refers to a nucleic acid sequence encoding easily assayed proteins. Among the more commonly used reporter genes are those for the following “reporter proteins”: chloramphenicol acetyltransferase (CAT), secreted alkaline phosphatase (SEAP), β-galactosidase (GAL), β-glucuronidase (GUS), luciferase (LUC), and green fluorescent protein (GFP). One of ordinary skill in the art will be aware of other available reporter genes.

The present invention also provides “reporter gene assays” as a method by which the transcriptional activity of a particular synthetic promoter construct within a cell can be analysed. These assays may comprise linking a reporter gene for visualizing the promoter activity, downstream of a promoter of interest to thereby obtain a reporter construct, introducing the reporter construct in a test cell, and quantifying the condition of promoter activation on the basis of the level of expressed reporter protein measured.

The reporter gene may, for example encode a secretable reporter protein, which when transcribed and translated, will result in the secretable reporter protein being synthesized and secreted into the external culture medium. The presence of the reporter molecule is monitored by assaying the culture medium without requiring the destruction or rupture of the microorganism host cells. An aliquot of culture medium is evaluated by any means capable of detecting the reporter molecule. Such means may either be, for example, by immunoassay or by other means known to the art. The rate of accumulation of the reporter molecule in the external culture medium is therefore an indication of the transcription activity of any promoter construct which was present on the fragment cloned adjacent and preceding the reporter gene sequences on the plasmid/expression vector.

Alternatively, if a visually identifiable reporter gene such as luciferase is used (which results in the emission of a photon in the presence of the substrate luciferin and ATP), the expression levels of the reporter protein can be easily monitored using a luminometer.

In one embodiment the recombinant protein is not a reporter protein, i.e. the construct of the present disclosure does not comprise a reporter gene.

In one embodiment the recombinant protein is not luciferase, i.e. the construct of the present disclosure does not comprise a gene encoding luciferase.

The term “transcriptional activity” as used herein refers to the transcription of the information encoded in DNA into a molecule of RNA, or the translation of the information encoded in the nucleotides of a RNA molecule into a defined sequence of amino acids in a protein.

A promoter construct with a “high” transcriptional activity refers to a construct which is able to express a recombinant protein or reporter protein at a “high” level of expression, defined as a level of expression that is higher than the mean level of expression obtained across a range of promoter constructs. As a reference point, synthetic promoters with the highest levels of activity exceed the activity of hCMV-IE1, which is widely acknowledged as one of the strongest promoters in CHO cells.

Conversely, a promoter construct with a “low” transcriptional activity refers to a construct which expresses a recombinant protein or reporter protein at a “low” level of expression, defined as a level of expression that is lower than the mean level of expression obtained across a range of promoter constructs.

Accordingly, the terms “transcriptional activity” and “expression level” are used interchangeably within the present specification.

The terms “oligonucleotide”, “polynucleotide” or “nucleotide sequence” are used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence or polyribonucleic acid sequence, or the like, and can be single stranded or double stranded, as well as a DNA/RNA hybrid. Furthermore, the terms “oligonucleotide”, “polynucleotide” and “nucleotide sequence” include naturally occurring nucleic acid molecules, which can be isolated from a cell, as well as synthetic molecules, which can be prepared, for example, by methods of chemical synthesis or by enzymatic methods such as by the polymerase chain reaction (PCR).

Synthetic methods for preparing a nucleotide sequence include, for example, the phosphotriester and phosphodiester methods (see Narang et al., Meth. Enzymol. 68:90, (1979); U.S. Pat. No. 4,356,270, U.S. Pat. No. 4,458,066, U.S. Pat. No. 4,416,988, U.S. Pat. No. 4,293,652; and Brown et al, Meth. Enzymol. 68:109, (1979), each of which is incorporated herein by reference). In various embodiments, an oligonucleotide of the invention or a polynucleotide useful in a method of the invention can contain nucleoside or nucleotide analogs, or a backbone bond other than a phosphodiester bond.

The nucleotides comprising an oligonucleotide (polynucleotide) generally are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al., Nucl. Acids Res. 22:5220-5234 (1994); Jellinek et al, Biochemistry 34:11363-11372 (1995); Pagratis et al., Nature Biotechnol. 15:68-73 (1997), each of which is incorporated herein by reference).

The covalent bond linking the nucleotides of an oligonucleotide or polynucleotide generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tarn et al., Nucl. Acids Res. 22:977-986 (1994); Ecker and Crooke, BioTechnology 13:351360 (1995), each of which is incorporated herein by reference). The incorporation of non-naturally occurring nucleotide analogs or bonds linking the nucleotides or analogs can be particularly useful where the nucleotide sequence is to be exposed to an environment that can contain a nucleolytic activity, including, for example, a tissue culture medium or upon administration to a living subject, since the modified nucleotide sequences can be less susceptible to degradation.

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally are chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995).

The term “library” as used herein where the context dictates refers to two or more TFRE constructs, two or more synthetic promoters or two or more expression vectors of the present disclosure or two or more cells of the present disclosure. As described throughout the specification, the term “library” is used in its broadest sense and may also encompass sub-libraries that may or may not be combined to produce the libraries of the present disclosure. TFREs identified by the methods of the invention as being active in a cell or tissue type of interest may be used to target genes to that cell or tissue type. For example, where the methods of the invention show that a TFRE is active specifically in a particular cell type, but not in a control cell type, then that TFRE may be used to specifically direct expression in the cell type of interest. Thus, a TFRE of the invention may be combined with a gene that it is desired to express in a particular cell type.

The term “comprising”, within in the context of the present specification, is intended to meaning “including”.

The term “about” or “approximately” means within 20%, preferably within 10%, and more preferably within 5% of a given value or range.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

The invention will now be described with reference to the following examples, which are merely illustrative and should not in any way be construed as limiting the scope of the present invention.

### REFERENCES

Al-Fageeh M B, Marchant R J, Carden M J, Smales C M. 2006. Biotechnology and bioengineering 93(5):829-835.
Blazeck J, Garg R, Reed B, Alper H S. 2012. Biotechnology and bioengineering 109(11):2884-2895.
Brown A J, Mainwaring D O, Sweeney B, James D C. 2013. Analytical biochemistry 443(2): 205-210. Dale L. 2006. BioProcess International 4:14-22.
Daramola O, Stevenson J, Dean G, Hatton D, Pettman G, Holmes W, Field R (2013) Biotechnology Progress Volume 30, Issue 1, pages 132-141, January/February 2014.
Datta P, Linhardt R J, Sharfstein S T. 2013. Biotechnology and Bioengineering Volume 110, Issue 5, pages 1255-1271, May 2013.
Ferreira J P, Overton K W, Wang C L. 2013. Proceedings of the National Academy of Sciences 110(28):11284-11289.
Ferreira J P, Peacock R W, Lawhorn I E, Wang C L. 2011. Systems and synthetic biology 5(3-4):131-138.
Girod P A, Zahn-Zabal M, Mermod N. 2005. Biotechnology and bioengineering 91(1):1-11.
Grabherr M G, Pontiller J, Mauceli E, Ernst W, Baumann M, Biagi T, Swofford R, Russell P, Zody M C, Di Palma F. 2011. Exploiting nucleotide composition to engineer promoters. PloS One 6(5):e20136.
Hai T, Curran T. 1991. Proceedings of the National Academy of Sciences 88(9):3720-3724.
Ho S C, Koh E Y, van Beers M, Mueller M, Wan C, Teo G, Song Z, Tong Y, Bardor M, Yang Y. 2013. Journal of biotechnology 165(3-4):157-166.
Kim M, O'Callaghan P M, Droms K A, James D C. 2011. Biotechnology and bioengineering 108(10):2434-2446.
A. M. Lanza, J. K. Cheng, and H. S. Alper, Current Opinion in Chemical Engineering (2012).
Le H, Vishwanathan N, Kantardjieff A, Doo I, Srienc M, Zheng X, Somia N, Hu W-S. 2013. Metabolic Engineering. 2013 November; 20:212-20.
Mader A, Prewein B, Zboray K, Casanova E, Kunert R. 2012. Applied microbiology and biotechnology:1-6.
Manke T, Roider H G, Vingron M. 2008. Statistical modeling of transcription factor binding affinities predicts regulatory interactions. PLoS Comput Biol 4(3):e1000039.
McLeod J, O'Callaghan P M, Pybus L P, Wilkinson S J, Root T, Racher A J, James D C. 2011. Biotechnology and bioengineering 108(9):2193-2204.
O'Callaghan P M, McLeod J, Pybus L P, Lovelady C S, Wilkinson S J, Racher A J, Porter A, James D C. 2010. Biotechnology and bioengineering 106(6):938-951.
Ogawa R, Kagiya G, Kodaki T, Fukuda S, Yamamoto K. 2007. BioTechniques 42(5):628.
Pasotti L, Politi N, Zucca S, De Angelis M G C, Magni P. 2012. Bottom-up engineering of biological systems through standard bricks: A modularity study on basic parts and devices. PloS One 7(7):e39407.
Prentice H, Tonkin C, Caamano L, Sisk W. 2007. Journal of biotechnology 128(1):50-60.
Schlabach M R, Hu J K, Li M, Elledge S J. 2010. Proceedings of the National Academy of Sciences 107(6):2538.
Schug J. 2008. Curr. Protoc. Bioinform. 21: 2.6.1-2.6.15
Stadlmayr G, Mecklenbräuker A, Rothmüller M, Maurer M, Sauer M, Mattanovich D, Gasser B. 2010. Journal of biotechnology 150(4):519-529.
Stinski M F, Isomura H. 2008. Medical microbiology and immunology 197(2):223-231.
Tornøe J, Kusk P, Johansen T E, Jensen P R. 2002. Gene 297(1):21-32.
Yim S S, An S J, Kang M, Lee J, Jeong K J. 2013. Biotechnology and bioengineering 110(11):2959-2969
Zhou H, Liu Z-g, Sun Z-w, Huang Y, Yu W-y. 2010. Journal of biotechnology 147(2):122-129.

### Example 1

### Identification of Transcription Factor Regulatory Elements that are Active in CHO-S Cells

In Silico Analysis of Transcription Factor Regulatory Elements

In order to identify discrete TFREs (transcription factor binding sites) capable of recombinant gene transactivation in CHO-S cells, the inventors surveyed for putative TFREs in ten viral promoters generally known to be active in CHO cells.

The following promoter sequences were retrieved from GenBank: hCMV-IE1 (accession number M60321.1), mouse CMV-IE1 (M11788), rat CMV-IE1 (U62396), guinea pig CMV-IE1 (CS419275), mouse CMV-IE2 (L06816.1), simian virus 40 early promoter and enhancer (NC_001669.1), adenovirus major late promoter (KF268310), myeloproliferative sarcoma virus long terminal repeat (LTR) (K01683.1), rous sarcoma virus LTR (J02025.1), and human immunodeficiency virus LTR (K03455.1).

Promoters were analysed using the Transcription Element Search System (TESS: http://www.cbil.upenn.edu/cgi-bin/tess/tess) and the Transcription Affinity Prediction tool (TRAP: http://trap.molgen.mpg.de/cgi-bin/trap_form.cgi) according to the methods previously described by Schug (Schug 2008) and Manke et al (Manke et al. 2008). Stringent search parameters were used to minimize false positives.

Using online search tools that scan DNA sequences for transcription factor (TF) binding sites, specifically Transcription Element Search System (TESS) and Transcription Affinity Prediction tool (TRAP), stringent search parameters (Manke et al. 2008; Schug 2008) were employed to minimize false positives. Across all viral promoter sequences, 67 discrete TFREs were identified as being present in one or more promoters. To further minimize this pool (design space) TFREs that did not occur in at least two promoters were filtered out. Based on the above in silico analysis, 28 transcription factor regulatory elements (TFREs) (see Table 2 below) were identified.

TABLE 2
Transcription factor regulatory elements identified by bioinformatic
survey of viral promoters: Ten viral promoters known to exhibit activity
in CHO cells were surveyed for the presence of discrete transcription
factor regulatory elements (transcription factor binding sites) using
Transcription Element Search System (TESS) and Transcription Affinity
Prediction (TRAP) algorithms using stringent search parameters to
minimize false positives. 28 single TFREs that occur in more than
one viral promoter are listed.

| | Transcription Factor |
|---|---|
| Promoter | Regulatory Elements |
| Human Cytomegalovirus | AP1, CArG, C/EBPα, CRE, |
| immediate early 1 | E4F1, EGR1, GC-box, Gfi1, |
| (hCMV-IE1) | IPF1, NF1, NFκB, RARE, |
| | YY1 |
| Mouse Cytomegalovirus | AP1, CRE, E-box, E4F1, |
| immediate early 1 | ERRE, Gfi1, HRE, IPF1, |
| (mCMV-IE1) | NF1, NFκKB, NFAT, NBRE, |
| | RARE |
| Rat Cytomegalovirus | AP1, E2F, ERRE, ISRE, |
| immediate early 1 | NFκB, NFAT, NBRE, RARE |
| (rCMV-IE1) | |
| Guinea pig Cytomegalovirus | AP1, GATA, GC-box, GRE, |
| immediate early 1 | HNF, MSX, NF1, NFκB, |
| (gpCMV-IE1) | OCT, RARE, YY1 |
| Mouse Cytomegalovirus | CArG, GC-box, cMyb, E2F, |
| immediate early 2 | EGR1, GATA, HRE, MSX, |
| (mCMV-IE2) | RARE |
| Simian virus 40 early | AP1, C/EBPα, cMyb, |
| promoter and enhancer | E-box, GATA, GC-box, |
| (SV40E) | MSX, NFκB, OCT |
| Adenovirus major late | CDP, E-Box, EGR1, GATA, |
| promoter (AdMLP) | GC-box, HNF, NF1, YY1 |
| Myeloproliferative | CDP, cMyb, ERRE, GATA, |
| sarcoma virus long | GC-box, Gfi1, |
| terminal repeat (LTR) | GRE, NF1, RARE, YY1 |
| (MPSV LTR) | |
| Rous sarcoma virus | CArG, CDP, C/EBPα, |
| LTR (RSV LTR) | ISRE, OCT |
| Human immunodeficiency | E-box, GC-box, GATA, |
| virus LTR (HIV LTR) | HNF, NFκB, NF1 |

### TFRE-Reporter Vector Construction

Previously described (Brown et al. 2013) promoter-less reporter-vectors (subcloned from pSEAP2 control (Clontech, Oxford, UK)) were utilized in this study. These plasmids contain a minimal hCMV-IE1 core promoter (5′-AGGTCTATATAAGCAGAGCTCGTTTAGTGA ACCGTCAGATCGCCTAGATACGCCATCCACGCTGTTTTGACCTCCATAGAAGAC-3′) (SEQ ID NO:170) upstream of either the secreted alkaline phosphatase (SEAP) or turbo green fluorescent protein (GFP) open reading frame (ORF).

To create RE reporter plasmids, synthetic oligonucleotides containing 7× repeat copies of each of the TFRE consensus sequences in Table 1 were synthesized (Sigma), PCR amplified, and inserted into KpnI and XhoI sites upstream of the CMV core promoter. Three clonally derived plasmids for each TFRE reporter were purified using a Qiagen plasmid mini kit (Qiagen, Crawley, UK). The sequence of all the plasmid constructs was confirmed by DNA sequencing.

### Cell Culture and Transfection

CHO-S and CHO-K1 cells were cultured in CD-CHO medium (Life Technologies) supplemented with 8 mM and 6 mM L-glutamine (Sigma) respectively. CHO-DG44 cells were cultured in CD-DG44 medium (Life Technologies) supplemented with 8 mM L-glutamine and 18 mL/L pluronic F68 (Life Technologies). All cells were routinely cultured at 37° C. in 5% (v/v) CO₂ in vented Erlenmeyer flasks (Corning, UK), shaking at 140 rpm and subcultured every 3-4 days at a seeding density of 2×10⁵ cells/ml. Cell concentration and viability were determined by an automated Trypan Blue exclusion assay using a Vi-Cell cell viability analyser (Beckman-Coulter, High Wycombe, UK). Two hours prior to transfection, 2×10⁵ cells from a mid-exponential phase culture were seeded into individual wells of a 24 well plate (Nunc, UK). Cells were transfected with DNA-lipid complexes comprising DNA and Lipofectamine (Life Technologies), prepared according to the manufacturer's instructions. Transfected cells were incubated for 24 h prior to protein expression analysis.

### Quantification of Reporter Expression

SEAP protein expression was quantified using the Sensolyte pNPP SEAP colorimetric reporter gene assay kit (Cambridge Biosciences, Cambridge, UK) according to the manufacturer's instructions. GFP protein expression was quantified using a Flouroskan Ascent FL Flourometer (Excitation filter: 485 nm, Emission filter: 520 nm). Background fluorescence/absorbance was determined in cells transfected with a promoter-less vector. The results of the reporter assays are shown in FIG. 1. Negligible basal activity was observed with CMV core promoter (−34 to +48 relative to TSS, includes TATA box and initiator element).

As can be seen, the majority of the TFREs did not result in an increased expression level of SEAP/GFP compared to when the core promoter alone was used. However, 7 TFREs, i.e. NFκB-RE, E-box, AP1-RE, CRE, GC-box, E4F1-RE and C/EBPα-RE showed higher expression levels of SEAP/GFP compared to the core promoter CMV alone.

These 7 TFREs were selected for incorporation into the Generation 1 synthetic promoters.

### Example 2

### Construction and Analysis of Generation 1 Promoters

### Synthetic Promoter Library Construction

Synthetic promoter TFREs were constructed from complementary single stranded 5′ phosphorylated oligonucleotides (Sigma, Poole, UK), annealed in STE buffer (100 mM NaCl, 50 mM Tris-HCl, 1 mM EDTA, pH 7.8, Sigma) by heating at 95° C. for 5 min, prior to ramp cooling to 25° C. over 2 h. Oligonucleotides were designed such that the resulting double stranded blocks contained the specific TFRE (Table 1) and a 4 by TCGA single stranded overhang at each 5′ termini. For example the sequences used for the NFkB-RE block were as follows (RE site underlined): 5′-TCGATGGGACTTTCCA-3′ SEQ ID NO: 171 and 5′-TCGATGGAAAGTCCCA-3′ SEQ ID NO: 172.

In order to construct a first generation synthetic promoter library, all 7 TFREs identified as transcriptionally active in CHO-S cells were used. Oligonucleotide building blocks containing a single copy of each TFRE sequence were chemically synthesized. NFκB, CRE, E-box, GC-box, E4F1, and C/EBPα; AP1 was omitted from the library due to previously observed functional redundancy between CRE and AP1 sites (Hai and Curran 1991). Transcription factor regulatory element blocks were synthesised by ligating TFREs at appropriate stoichiometric molar ratios with high concentration T4 DNA ligase (Life Technologies, Paisley, UK). A ‘cloning-block’ containing KpnI and XhoI sites was included in ligation mixes at a 1:20 molar ratio of the TFREs. The ligated molecules were digested with KpnI and XhoI (Promega, Southampton, UK), gel extracted (Qiaquick gel extraction kit, Qiagen), and inserted upstream of the minimal CMV core promoter in the promoter-less SEAP reporter vector. Clonally derived plasmids were purified and sequenced. A control CMV promoter reporter plasmid was constructed using the hCMV-IE1 promoter (hereafter referred to as CMV) upstream of the SEAP ORF.

Transient production of SEAP was employed to determine the relative activity of synthetic promoters as it both maximises throughput and provides a direct readout of synthetic promoter transactivation without potential interference from integration-specific effects or silencing. Whilst SEAP production is not a direct measurement of transcriptional activity, previous experiments in this laboratory have confirmed that SEAP activity in cell culture supernatant is linearly correlated with SEAP mRNA levels post-transfection. Moreover, assay conditions were optimized such that control CMV-SEAP reporter activity was in the centre of the linear assay range with respect to plasmid copy number (DNA load) and measured SEAP output (data not shown).

Purified plasmid DNA from 110 transformed E. coli colonies picked at random was utilized for measurement of SEAP reporter production. SEAP production at 24 h post transfection was measured for each synthetic promoter, and each promoter was sequenced to reveal its TFRE-block composition. A small proportion (14) of reporter plasmids were found to be lacking a promoter insert and these were excluded from further analysis.

The relative transcriptional activity of the remaining 96 promoters is shown in FIG. 2, and their TFRE-block compositions are listed in Table 3 below. These data show that generation 1 synthetic promoter activities spanned two orders of magnitude, where the most active synthetic promoter exhibited a 1.2-fold increase in SEAP production over that deriving from the CMV control vector.

TABLE 3
Generation 1 Promoters: N = NFκB-RE, E = E-box, G = GC-box, B = C/EBPα-
RE, C = CRE, F = E4F1-RE. TFREs in the reverse orientation (i.e. 3′ to
5′ with respect to the SEAP reporter) are indicated by an apostrophe.
All other TFREs are in the 5′ to 3′ orientation.

| Promoter | | Relative | SEQ ID |
|---|---|---|---|
| Name | Promoter Sequence | Activity (%) | No: |
| 1/01 | N E E N G B C E′ B′ N N E G′ N | 100 | 30 |
| 1/02 | E′ F F′ B′ E G′ N′ E′ B N N′ N N′ G N | 84.46 | 31 |
| 1/03 | E E G′ C N′ G′ G G′ C′ E N N′ B′ E′ B E′ | 67.20 | 32 |
| 1/04 | B′ C′ E′ G C B′ N N′ N′ G F′ B N′ | 58.3 | 33 |
| 1/05 | G F N G N B′ N′ B C E′ N′ | 56.87 | 34 |
| 1/06 | C E E′ G′ E N E C B′ G′ C′ N′ G′ | 54.89 | 35 |
| 1/07 | B′ E B′ G′ N B B E C′ N N | 54.71 | 36 |
| 1/08 | E E′ N′ C B′ E′ E G B E N | 52.64 | 37 |
| 1/09 | B G′ N B′ C′ N′ E E′ C′ G E′ | 51.31 | 38 |
| 1/10 | B′ E N′ E E′ B C′ G G N | 48.83 | 39 |
| 1/11 | C′ E′ N B′ E G′ F′ | 47.64 | 40 |
| 1/12 | B′ N N E E G′ C E N′ B′ B | 47.18 | 41 |
| 1/13 | G B E′ N′ G E N C′ B′ N E′ E′ C E′ | 44.7 | 42 |
| 1/14 | C G G N C B B′ N′ N C′ F E′ N | 38.43 | 43 |
| 1/15 | N′ B F′ C′ N′ B E′ N C′ N′ | 35.94 | 44 |
| 1/16 | C F′ N N B′ C′ E E′ F′ | 34.31 | 45 |
| 1/17 | G′ G G B′ E N B N′ B′ C B′ E G′ G | 34.27 | 46 |
| 1/18 | N N′ E B′ C G′ E B′ C′ | 34.03 | 47 |
| 1/19 | E E C E C N N′ F C N B N′ E′ | 33.77 | 48 |
| 1/20 | F′ G E E E′ N′ F E B G′ E′ E | 33.35 | 49 |
| 1/21 | N E N′ E′ N N N′ | 32.57 | 50 |
| 1/22 | F G′ B′ E N′ E′ B′ E′ G G B | 31.73 | 51 |
| 1/23 | C′ N E′ B C B N G′ E | 30.32 | 52 |
| 1/24 | C N′ B B E′ F N E′ G N′ C | 28.25 | 53 |
| 1/25 | G G′ B′ G′ B′ N′ C N N B′ E′ C C E′ B′ | 28.13 | 54 |
| 1/26 | N G′ B′ N E E E′ F′ N B′ N′ B | 27.33 | 55 |
| 1/27 | B′ C E F′ B′ E F′ N G N N | 26.87 | 56 |
| 1/28 | E′ F N′ N E E C N E′ F′ N G′ N G E′ F | 25.72 | 57 |
| | N G′ N E′ F′ | | |
| 1/29 | N N N N N N | 25.69 | 58 |
| 1/30 | C B′ N E′ E′ G N′ | 24.68 | 59 |
| 1/31 | B′ G B E′ F′ N′ C B N′ E′ B G | 23.33 | 60 |
| 1/32 | E′ F G′ N G E E N F N C G B′ G G B′ | 20.09 | 61 |
| 1/33 | C N′ F N G′ G′ G′ N′ F F C′ N E′ N′ | 19.46 | 62 |
| 1/34 | G G′ N′ E E F C E B′ E′ C′ N′ F G′ | 15.22 | 63 |
| 1/35 | C B C′ E B G′ N′ B′ E′ G N′ G G | 15.16 | 64 |
| 1/36 | E C′ N G F′ F G F G′ N E B C′ | 14.48 | 65 |
| 1/37 | N F′ E E F F′ B N B N F G F′ C C′ G N′ | 14.44 | 66 |
| | E C N′ | | |
| 1/38 | N′ C N C C E′ G C B′ N N | 13.75 | 67 |
| 1/39 | C′ E′ C′ E′ F B G′ G′ N′ G E′ E′ CC | 13.59 | 68 |
| 1/40 | E′ F G C E F F′ G′ G F′ G F G E G B′ | 13.57 | 69 |
| | N B′ E N F B G′ | | |
| 1/41 | B′ B′ E G′ F′ G′ B′ F′ E N′ B C | 12.38 | 70 |
| 1/42 | F′ B′ G′ N B B F′ E′ E B C′ N B E′ N G′ | 12.23 | 71 |
| | B′ C N′ G | | |
| 1/43 | G E′ F N C B C′ E E | 12.1 | 72 |
| 1/44 | G′ E E F C′ C′ B′ N E′ | 12.04 | 73 |
| 1/45 | F′ B N′ C′ B C′ G N′ E′ G E C | 12.01 | 74 |
| 1/46 | N G E′ C′ G′ E B E′ E C F C′ E′ E F E G | 11.56 | 75 |
| | C | | |
| 1/47 | E E E E E E E | 10.78 | 76 |
| 1/48 | E′ G′ F G′ E′ C F G′ E′ G B′ E′ B B C | 9.11 | 77 |
| 1/49 | B′ E′ F G′ N F C G G′ B′ N′ B′ E C | 8.94 | 78 |
| 1/50 | F G′ E′ E′ C′ B′ | 8.74 | 79 |
| 1/51 | F C′ G′ N F′ C G N G C′ B E G′ | 8.53 | 80 |
| 1/52 | C N′ C′ N C′ G′ N′ E′ N′ F C C N′ B′ C′ | 8.53 | 81 |
| | C N C′ N′ F G | | |
| 1/53 | G G′ F′ N B′ N′ F′ E′ B′ N | 8.44 | 82 |
| 1/54 | C C C C C C C | 8.43 | 83 |
| 1/55 | G′ C C′ C N′ E N | 8.03 | 84 |
| 1/56 | G′ B′ C′ N E′ G E F′ F | 7.38 | 85 |
| 1/57 | N′ B′ B G′ F G′ F N E | 6.89 | 86 |
| 1/58 | N G C′ C | 7.74 | 87 |
| 1/59 | C′ G′ G E′ E′ G′ B N B N F′ F E F′ | 6.06 | 88 |
| 1/60 | G G G G G G G | 6.03 | 89 |
| 1/61 | G′ B′ C C N′ B′ E E B′ | 5.94 | 90 |
| 1/62 | F F F F F F | 4.7 | 91 |
| 1/63 | E′ E G G C′ N′ G B′ C G F′ F′ B E E B F′ | 4.62 | 92 |
| 1/64 | E B B C′ F′ C B E′ G′ | 4.29 | 93 |
| 1/65 | F′ B G′ B′ C E E′ F′ B | 4.04 | 94 |
| 1/66 | E′ E C G N C′ F′ E C G′ E F′ N C | 4 | 95 |
| 1/67 | E′ G′ G′ N F E′ F′ N N′ G F F′ | 3.94 | 96 |
| 1/68 | G′ G′ N′ F′ N′ C B′ E′ C′ E B F E′ F N′ | 3.57 | 97 |
| | G C′ G C′ N G′ C B C F N′ B E F B′ G | | |
| 1/69 | E G B C F′ B B G N C | 3.43 | 98 |
| 1/70 | E′ N′ G F N E′ G′ C F E′ C′ G G C B′ | 3.35 | 99 |
| 1/71 | E′ N′ F G′ C F E B C′ C N | 3.24 | 100 |
| 1/72 | F F′ C G′ C′ B′ C′ N′ C G B B F′ C N′ | 3.14 | 101 |
| | F E′ E B′ F′ G′ G B′ | | |
| 1/73 | B B B B B B B | 3.13 | 102 |
| 1/74 | C′ G′ C′ B′ B E B′ C′ B B B | 3.1 | 103 |
| 1/75 | G G N′ N′ F G F C′ B G′ | 2.57 | 104 |
| 1/76 | B F G G G G F B C′ E F | 2.52 | 105 |
| 1/77 | E F E′ N F G′ N F N G′ F C′ F G′ G | 2.46 | 106 |
| 1/78 | F′ G G C′ G G′ F N′ B′ | 2.39 | 107 |
| 1/79 | F F F′ E′ F′ C′ F B C′ N B E B′ | 2.22 | 108 |
| 1/80 | E′ G C E F′ F′ B′ G E′ G | 2.11 | 109 |
| 1/81 | B′ G C B′ G F G F′ | 2.02 | 110 |
| 1/82 | B′ E′ B′ B G′ B F′ C′ B′ N C′ C′ G′ | 2.01 | 111 |
| 1/83 | F′ C′ F′ N B G′ N G′ | 2 | 112 |
| 1/84 | B′ C′B′ F′ G′ G E′B C G C | 1.94 | 113 |
| 1/85 | C′ E E′ E B G F G F′ F F′ E G | 1.87 | 114 |
| 1/86 | G′ E′ N F E B′ F C′ E′ F′ C C′ | 1.71 | 115 |
| 1/87 | F′ C B′ G F G G E F′ C′ E | 1.57 | 116 |
| 1/88 | C′ N′ G E′ C′ G C N′ G′F F | 1.35 | 117 |
| 1/89 | C′ G G B′ C C B′ | 1.34 | 118 |
| 1/90 | F C′ G′ B′ F E′ F′ G′ F G C | 1.33 | 119 |
| 1/91 | E′ B′ C E F′ F′ F B B G′ B′ C C B C′ G | 1.33 | 120 |
| 1/92 | C′ E′ F C F′ F′ E F′ E B | 1.26 | 121 |
| 1/93 | B F′ G E G′ G N F C F | 1.23 | 122 |
| 1/94 | B′ E G N′ N F C G F N F C′ F | 0.66 | 123 |
| 1/95 | G′ G′ N′ F′ F′ E′ C′ B′ C C′ | 0.38 | 124 |
| 1/96 | E B E′ B′ E E B′ F G C′ F F B | 0.36 | 125 |

Analysis of synthetic promoter composition revealed that (i) synthetic promoter length varied between 7 and 31 TFREs (mean=11.9±4.2 blocks; 189±66 bp), although relative transcriptional activity was unrelated to promoter length (ii) across the generation 1 library the relative abundance of the six TFREs was approximately equivalent and (iii) individual TFREs could occur in either forward or reverse orientation [i.e. the consensus TF recognition sequence (see Table 1) could occur on either DNA strand] but this was not apparently related to synthetic promoter activity, either with respect to the general frequency of occurrence or with respect to the relative orientation of specific TFRE blocks.

Thus, the inventors inferred that variation in synthetic promoter activity was a consequence of the differing relative abundance of specific TFREs within promoters and/or positional effects (i.e. that specific neighbouring or distal combinations of TFREs may affect promoter strength). Whilst the latter is computationally intractable given the size of the library, the inventors addressed the former by determination of the relative frequency with which individual TFREs occurred within synthetic promoters of varying activity. These data are shown in FIG. 3.

Whilst no single TFRE exhibited an obviously dominant influence over synthetic promoter strength, individual TFREs were either relatively abundant in high transcriptional activity promoters (NFκB, E-box), equally distributed across promoters (C/EBPα, GC-box) or relatively abundant in low activity promoters (E4F1, CRE). This bias was confirmed by multiple linear regression analysis, where either an all factor model (inclusion of all six TFREs, r²=0.57, p=1.7×10⁻¹⁴) or a parsimonious model excluding C/EBPα and GC-box TFREs (as these do not improve model fit; r²=0.56, p=8.84×10⁻¹⁶) predicted the optimal stoichiometry of TFRE blocks to be NFκB 1.58: E-box 1.

The other TFREs were either neutral (C/EBPα, GC-box) or negative effectors (E4F1, CRE). Analysis of specific promoter sequences throughout the library confirmed site designations as positive, neutral or negative. For example, the strongest promoter (1/01) contains the highest ratio of positive (NFκB, E-box): negative (E4F1, CRE) sites (9:1) in the library. Moreover, the three most active promoters (1/01-1/03) are the only promoters in the library containing more than 7 positive sites and less than 3 negative sites. There are also multiple examples where high numbers of positive sites are apparently counteracted by high numbers of negative sites to produce relatively weak promoters, for example promoters 1/37, 1/52 and 1/68 which have positive:negative ratios of 8:8, 8:8, and 9:11 respectively.

Accordingly, this Example demonstrates that the synthetic promoters of the present invention have the potential to tailor the expression of a recombinant protein to a specific expression level depending on the promoter construct selected.

### Example 3

### Construction and Analysis of Generation 2 Synthetic Promoters

Based on the above analysis of the Generation 1 synthetic promoters, the inventors sought to further improve synthetic promoter activity by creating a Generation 2 synthetic promoter library using random ligation of a mixture of TFREs at an optimal ratio derived from analysis of the composition of Generation 1 promoters.

Four of the initial 7 TFREs identified (see FIG. 1) were utilised for construction of a Generation 2 library of synthetic promoter constructs at a stoichiometry quantitatively derived from their relative representation in active synthetic promoters in the Generation 1 synthetic promoter constructs. The stoichiometric ratios used were 5:3:1:1 (NFkB-RE:E-box: C/EBPα-RE:GC box).

Specifically, of the initial 7 TFREs, the negative TFREs, E4F1 and CRE (see FIG. 3) were omitted (i.e. promoters which contained larger numbers of these TFREs were associated with lower reporter gene expression levels), whilst the neutral TFREs, C/EBPα and GC-box were included based on the hypothesis that increased complexity could be advantageous. For example, the three most active synthetic promoters in the first generation library all contained at least two copies of both neutral TFREs (see Table 3) and thus they could contribute to unknown positional effects. The inventors expected that second generation promoters would contain the same average number of TFREs (12) as first generation promoters.

The Generation 2 promoter constructs (see Table 4 below for sequences) were generated using the same construction method described above in Example 2.

TABLE 4
Generation 2 Promoters: N = NFκB-RE, E = E-box,G = GC-box, B = C/EBPα-RE.
TFREs in the reverse orientation (i.e. 3′ to 5′ with respect to
the SEAP reporter) are indicated by an apostrophe. All other TFREs are in
the 5′ to 3′ orientation.

| | | Relative | |
|---|---|---|---|
| | | Activity | |
| | | (as a % of | |
| Promoter | | hCMV-IE1 | SEQ ID |
| Name | Promoter Sequence | activity) | NO: |
| 2/01 | N G E E′ N E′ E′ N N N E′ E N E′ | 216.66 | 126 |
| 2/02 | E N′ N′ N′ E E E′ N′ E′ B′ N′ E′ N N E′ | 175.94 | 127 |
| 2/03 | E E N′ G′ E N′ E′ N′ N′ E′ N′ N′ N B | 174.82 | 128 |
| 2/04 | N E N E N N′ N′ E N | 169.04 | 129 |
| 2/05 | N′ N G N B′ E′ E′ N B N E′ N N′ | 166.6 | 130 |
| 2/06 | N′ N′ N N E′ N G′ E′ N′ E B′ | 157.92 | 131 |
| 2/07 | N′ N′ E′ N G′ N′ N′ N′ B N | 155.3 | 132 |
| 2/08 | E′ E′ N′ B′ N′ N′ G′ N N N′ E | 154.47 | 133 |
| 2/09 | N′ N′ E N B N N′ E′ N G E′ E | 151.37 | 134 |
| 2/10 | E′ N B′ N′ N E′ N B N N N′ G′ N N N′ | 150.63 | 135 |
| 2/11 | E N′ E N N′ N′ N′ N E′ N′ N′ N′ N′ B N′ E′ | 150.17 | 136 |
| | N E′ N E′ | | |
| 2/12 | N′ E′ N N B′ N′ N N E′ B E | 148.93 | 137 |
| 2/13 | E B B′ E B′ N N E′ N N′ E E G′ N G N E B N | 143.62 | 138 |
| 2/14 | E N′ N′ N G N N′ G′ B N′ G N | 140.87 | 139 |
| 2/15 | N G′ E′ N′ N′ N N′ E′ N | 140.87 | 140 |
| 2/16 | N G′ E N N′ B′ N N G′ G | 140.77 | 141 |
| 2/17 | E′ N G′ G′ E′ E N N N E′ N N′ G′ N′ B′ N E | 138.95 | 142 |
| | E′ N N N | | |
| 2/18 | N′ N′ N N′ G′ N′ N G′ E′ N B E | 138.83 | 143 |
| 2/19 | E′ N′ N B′ N N E′ G N N E B′ N E | 137.6 | 144 |
| 2/20 | E E N′ E′ E′ E N′ G′ E′ N E′ N G′ | 137.53 | 145 |
| 2/21 | N N E′ E N′ N G E′ N′ E′ | 132.61 | 146 |
| 2/22 | N E′ G′ E N′ B E′ N′ E N | 117.77 | 147 |
| 2/23 | N′ E′ N′ N G N′ N N G E′ | 117.22 | 148 |
| 2/24 | B′ E N′ N′ E N′ G′ N E′ G N B′ N | 113.38 | 149 |
| 2/25 | N G′ N N N N′ N′ B N′ N | 108.35 | 150 |
| 2/26 | B E′ G′ N G′ N′ E G E′ N′ G′ | 99.36 | 151 |
| 2/27 | N′ N E′ N′ N′ E E B′ E G′ E′ | 98.83 | 152 |
| 2/28 | E N′ N′ N N′ N N′ B | 97.9 | 153 |
| 2/29 | B′ N′ G′ G′ E′ N′ E′ N E N′ BN | 94.66 | 154 |
| 2/30 | E N N′ N E N B N′ N′ | 94.36 | 155 |
| 2/31 | E′ G′ G′ N′ G′ E B N′ E N′ N | 90.93 | 156 |
| 2/32 | N N G′ E E′ N N′ G′ E E | 90.49 | 157 |
| 2/33 | N′ E G N N′ E E′ B′ N′ | 87.38 | 158 |
| 2/34 | B E G′ N′ N N G E | 82.11 | 159 |
| 2/35 | N′ N′ G B G′ B′ N N′ N N′ B′ | 76.61 | 160 |
| 2/36 | N′ E N′ G N N B′ E G | 75.11 | 161 |
| 2/37 | G′ N N N′ N E E G′ G′ | 70.59 | 162 |
| 2/38 | G E B N N E′ E′ N′ | 70.24 | 163 |
| 2/39 | N N G E′ B E′ N | 63.19 | 164 |
| 2/40 | G′ N′ N E′ G N′ N′ B G | 62.21 | 165 |
| 2/41 | E B′ E′ E B′ N′ N′ E′ B′ | 57.26 | 166 |
| 2/42 | N′ G N′ N E G B′ B′ | 41.65 | 167 |
| 2/43 | G E′ B′ N G′ N G B′ N | 36.61 | 168 |
| 2/44 | B E E′ E B E′ B′ B E′ N | 32.3 | 169 |

50 transformed E. coli colonies were picked at random, synthetic promoters in purified plasmid DNA were sequenced and 44 reporter plasmids containing promoter sequences were utilized for measurement of SEAP reporter production. The relative transcriptional activity of second generation promoters is shown in FIG. 4.

The Generation 2 synthetic promoters exhibited significantly increased activity compared to the Generation 1 promoters. In particular, the mean expression level (relative to CMV) shifted from 21.2% for first generation promoters to 116% for the second generation library. In fact, the results indicate that a large number of Generation 2 promoters have a higher activity than the top Generation 1 promoter. Twenty five Generation 2 synthetic promoters (57% of the library) achieved a higher SEAP production than the CMV control, with the strongest promoter (2/01) exhibiting a 2.2-fold increase.

FIG. 5 shows the results of an analysis of the relative abundance of each TFRE relative to the expression levels of the Generation 2 synthetic promoter constructs in which the TFREs are present.

Analysis of the TFRE block composition of the second generation promoters revealed that the relative stoichiometry of TFREs across the library was approximately as designed (NFκB 5:E-box 2.81:GC-box 1.32:C/EBPα1.15). As shown in FIG. 5, for second generation promoters the influence of GC-box and C/EBPα is generally negative, whereas NFκB and E-box remain positive effectors.

However, considering the composition of second generation promoters (see sequences in Table 4), the results suggest that neither NFκB nor E-box TFRE blocks could support high transcriptional activity alone—a combination of both is necessary.

The most powerful promoters (2/01-2/03) contain relatively high numbers of both TFREs in approximately equal proportion, with a correspondingly low number of negative GC-box and C/EBPα blocks. Some lower activity promoters do contain relatively large numbers of NFκB or E-box blocks (e.g. 2/11, 2/13, 2/17) but (i) contain a sub-optimal ratio of NFκB:E-box (2/11, 2/17) or (ii) also contain relatively large numbers of GC-box and C/EBPα blocks (2/13).

Therefore, this Example demonstrates that the Generation 2 promoters are able to successfully extend the possible transcriptional activity range that can be achieved in CHO cells using Generation 1 promoter constructs.

### Example 4

### Analysis of Generation 1 and 2 Promoter Constructs in Different CHO Host Cell Types

In order to determine if the synthetic promoters of the present invention performed robustly and predictably, the inventors evaluated their relative functional capability in different CHO host lines.

A panel of seven promoters from both first and second generation libraries were selected that cover a broad range of promoter activity (i.e. 1/51<1/17<1/04<1/02<2/19<2/03<2/01). These were compared to the activity of CMV.

FIG. 6 shows transient SEAP production from all promoters in three commonly utilized host lines; CHO-S, CHO-DG44 and CHO-K1. The relative rank order of promoter activity is maintained in all three cell lines, with the exception that 2/03 outperforms 2/01 in CHO-K1—in contrast to the original screen (see Example 3), promoters 2/03 and 2/01 have approximately equivalent expression in CHO-DG44 and CHO-S.

In each cell line, the top performing synthetic promoter drives significantly higher SEAP production than CMV—3.1-fold 1.9-fold, and 1.7-fold in CHO-DG44, CHO-S, and CHO-K1 cells respectively. In general, CHO-DG44 cells exhibited significantly less reporter production than either CHO-S or CHO-K1 cells, presumably due to their reduced “transfectability” by lipofection.

Nonetheless, this Example indicates that the rank order of the promoter constructs is generally preserved across different CHO cell types and suggests that the promoter constructs will also be effective in other CHO cell types besides the 3 types tested, and also suggests that the synthetic promoters are likely be functional in a range of transformed mammalian cell types and may have use in cancer-targeted gene therapeutic applications.

### Example 5

### Analysis of Generation 1 and 2 Promoter Constructs in Longer Term Transient Transfections

Next, to determine synthetic promoter functionality in an industrially relevant production process, the same panel of promoters was evaluated in a fed-batch SEAP production process over a longer term transient transfection (7 days), utilizing CHO-S host cells.

A transient system was employed (rather than stable) to ensure production variability was directly linked to differences in promoter activity rather than cell line specific, site-specific integration or promoter silencing (e.g. methylation, deletion) artefacts.

Two hours prior to transfection 6×10⁶ cells from a mid-exponential phase CHO-S culture were seeded into 50 mL CultiFlask bioreactors (Sartorius, Surrey, UK) at a working volume of 6 mL. Cells were transfected with DNA:lipofectamine complexes, prepared according to the manufacturer's instructions. Fed-batch cultures were maintained for seven days by nutrient supplementation with 10% v/v CHO CD Efficient Feed A (Life Technologies) on day 2, 4 and 6. SEAP expression and cell growth were measured at 24 h intervals.

The results of the SEAP expression assays are shown in FIG. 7. As can be seen, the relative order of activity is mostly preserved compared to the short term transient expression results (i.e. the static microplate experiments from Example 4, see FIG. 6), indicating that robust and reproducible expression levels can be achieved long term using the synthetic promoter constructs of the present invention.

In addition, the highest SEAP titer, driven by promoter 2/03, was over 1.65-fold that obtained by CMV-mediated expression.

The IVCD results further show that cell viability is not significantly affected when the CHO cells are transfected with the promoter constructs of the present invention. This is important because it shows that the promoter constructs have the potential to be used in host cells without adversely affecting normal cellular function.

1. A CHO cell, comprising a synthetic promoter suitable for eliciting recombinant protein expression therein, said synthetic promoter comprising a promoter core and upstream thereof two or more transcription factor regulatory elements independently selected from the group consisting of NFκB-RE, E-box, AP1, CRE, GC-Box, E41F, C/EBPα-RE, OCT and RARE.

2. The CHO cell according to claim 1, wherein the promoter core is selected from CMV, SV40, UbC, EF1A, PGK and CAGG.

3. The CHO cell according to claim 1, wherein the synthetic promoter comprises 2 to 50 transcription factor regulatory elements.

4. The CHO cell according to claim 1, wherein the transcription factor regulatory elements are all the same type.

5. The CHO cell according to claim 1, wherein the transcription factor regulatory elements are a combination of different types, which are, optionally, independently selected from NFκB-RE, E-box, GC-Box, C/EBPα-RE, CRE and E41F.

6. The CHO cell according to claim 1, wherein the transcription factor regulatory elements are arranged in tandem.

7. (canceled)

8. The CHO cell according to claim 1, wherein
a) synthetic promoter DNA sequence is 0.9 or less of the size of the full length CMV promoter sequence, and/or
b) the synthetic promoter has a transcriptional activity per unit DNA sequence thereof which in greater than the transcriptional activity per unit DNA of CMV promoter.

9. (canceled)

10. The CHO cell according to claim 1, wherein the CHO cell is selected from CHO-S, CHO-K1 and CHO-DG44.

11. The CHO cell according to claim 1, wherein the activity of the transcription factor regulatory element YY1 is inhibited, for example by a block decoy specific to YY1.

12. The CHO cell according to claim 1, wherein the cell further comprises a polynucleotide sequence encoding a recombinant protein under the control of the synthetic promoter, wherein, optionally, the recombinant protein is an antibody or antigen binding fragment thereof.

13. (canceled)

14. The CHO cell according to claim 1, wherein the promoter exhibits improved protein expression in comparison to the promoter core or the wild type promoter, wherein, optionally, the improved protein expression is a greater level of recombinant protein expression.

15. (canceled)

16. The CHO cell according to claim 1, wherein the synthetic promoter comprises a sequence given in any one of SEQ ID NOs: 30 to 169.

17. The CHO cell according to claim 16, wherein the synthetic promoter comprises
a) a sequence given in any one of SEQ ID NOs: 126 to 169, or
b) the nucleotide sequence given in SEQ ID NO:30, SEQ ID NO:31, SEQ ID NO:32, SEQ ID NO:126, SEQ ID NO:128 or SEQ ID NO:144.

18. (canceled)

19. The CHO cell according to claim 1, wherein the synthetic promoter
a) does not comprise any CpG islands, and/or
b) has properties suited to the expression of the specific recombinant protein it is associated with.

20. (canceled)

21. A synthetic promoter suitable for promoting recombinant protein expression in a CHO cell said synthetic promoter comprising a promoter core and upstream thereof a mixture of two or more transcription factor regulatory elements independently selected from the group consisting of NFκB-RE, E-box, AP1, CRE, GC-Box, E41F, C/EBPα-RE, OCT and RARE.

22. The synthetic promoter according to claim 21 wherein the transcription factor regulatory elements are independently selected from the group consisting of NFκB-RE and CRE.

23. A method of generating a synthetic promoter suitable for promoting recombinant protein expression in a given mammalian recombinant host cell comprising,
a) identifying motifs of transcription factor regulatory elements,
b) testing each transcription factor regulatory element identified in a) combined with a promoter core for activity in a chosen mammalian recombinant host cell line,
c) selecting two or more transcription factor regulatory elements from (b) which are more active in the chosen mammalian recombinant host cell line than the promoter core alone,
d) preparing one or more synthetic promoter constructs comprising two or more of those transcription factor regulatory elements independently selected from those selected in c),
e) testing the synthetic promoter construct or constructs prepared in d) for activity in the chosen mammalian recombinant host cell,
f) identifying the synthetic promoter construct or constructs that exhibit the same or improved protein expression compared to a wild type promoter
wherein the method optionally additionally comprises
g) selecting two or more of those transcription factor regulatory elements which are associated with the constructs identified in f), and
h) preparing one or more synthetic promoter constructs comprising a TFRE construct comprising or consisting of those elements independently selected in g).

24. (canceled)

25. The method according to claim 24, wherein the TFRE constructs prepared in step h comprise transcription factor regulatory elements at a stoichiometry which reflects their relative abundance in the constructs identified in f).

26. The method according to claim 23, wherein part (f) of the method further comprises identifying the transcription factor regulatory element or elements that are most frequently associated with promoter constructs which exhibit reduced protein expression compared to the wild type promoter and excluding these in (g).

27. A method of identifying a synthetic promoter suitable for promoting recombinant protein expression in a given mammalian recombinant host cell at a desired level comprising the steps of:
a) obtaining two or more synthetic promoter constructs defined in claim 21 or claim 22,
b) testing the synthetic promoter constructs obtained in a) to determine the level of recombinant protein expression driven by each construct in the chosen mammalian recombinant host cell,
c) selecting a synthetic promoter construct tested in (b) if it promotes recombinant protein expression at the desired level.

28. The method according to claim 27, wherein the two or more synthetic promoters obtained in step (a) each comprises a sequence independently selected from any one of SEQ ID NOs: 30 to 169 or SEQ ID NOs 126-169.

29. The method according to claim 27, wherein the desired level of protein expression is higher or lower than that achieved using a wild type promoter.

30. (canceled)

31. A method of constructing a transcription factor regulatory element construct library comprising the step of randomly ligating the transcription factor regulatory elements
a) NFκB-RE and E-box at a ratio of 5:3, or
b) NFκB-RE, E-box, GC-Box and C/EBPα-RE at a ratio of 5:3:1:1.

32. (canceled)

33. A CHO cell wherein the activity of the transcription factor regulatory element YY1 activity is knocked down or knocked out by a block decoy specific to YY1.

34. (canceled)

35. The CHO cell according to claim 33, wherein the cell further comprises a polynucleotide sequence encoding a recombinant protein under the control of the synthetic promoter.
